(19) United States
(12) Patent Application Publication
(10) Pub. No.: US 2004/0259888 A1
(43) Pub. Date: Dec. 23, 2004

(54) Novel 4-aminofuropyrimidines and the use thereof

(76) Inventors: Erwin Bischoff, Wuppertal (DE); Markus Hauswald, Koln (DE); Peter Nell, Wuppertal (DE); Susanne Rohrig, Essen (DE); Karl-Heinz Schlemmer, Wuppertal (DE); Henning Steinhagen, Sulzbach (DE); Jurgen Stoltefub, Haan (DE); Stefan Weigand, Wuppertal (DE)

Correspondence Address: Jeffrey M Greenman, Bayer Corporation, 400 Morgan Lane, West Haven, CT, 06516, US

(21) Appl. No.: 10/487,373

(22) PCT Filed: Aug. 9, 2002
(86) PCT No.: PCT/EP02/08906

(30) Foreign Application Priority Data
Aug. 22, 2001 (DE) 101 41 212.6

(51) Int. Cl. A61K 31/519; C07D 491/02

(52) U.S. Cl. 514/260.1; 544/279; 514/227.8; 514/234.2; 514/252.16

(57) ABSTRACT

The invention relates to compounds of formula (I), a method for the production thereof in addition to the use thereof as medicaments.

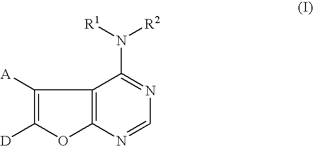

The invention relates to novel 4-aminofuro[2,3-d]pyrimidine derivatives, to processes for their preparation and to their use in pharmaceuticals, in particular for the prevention and/or treatment of cardiovascular disorders.

Adenosine is an endogenic cardioprotective and neuroprotective effector (Olafsson et al., Circulation 1987, 76:1135-1145; Dragunow and Faull, Trends in Pharmacol. Sci. 1988, 9:193; Marangos, Medical Hypothesis 1990, 32:45). It is released under hypoxic conditions, for example, in the case of cardiac or peripheral occlusion diseases (W. Makarewicz, “Purine and Pyrimidine Metabolism in Man”, Plenum Press, New York, 11, 1998, 351-357). Accordingly, this effect is particularly pronounced under cell-damaging conditions with limited oxygen supply, such as, for example, in the case of ischemia. Adenosine is a highly effective vasodilator and is involved in metabolic regulation of blood flow. It enhances ischemic preconditioning (R. Strasser, A. Vogt, W. Scharper, Z. Kardiologie 85, 1996, 79-89; Schrader, Circulation 1990, 81:389-391) and can promote the growth of collateral vessels. Accordingly, adenosine protects, as a natural defense mechanism, against the sequela of a number of pathophysiological ischemia-related situations, such as cerebral and cardinal ischemia, in particular by increasing coronary or cerebral perfusion by vasodilatation by inhibiting platelet aggregation and by stimulating angiogenesis. The many pharmacological effects of adenosine also include action on perfusion of the kidneys, on respiration, on pain, on inflammations, on the gastrointestinal tract, on blood cells and on adipocytes.

However, systemically administered adenosine has a very short half-life (Moser et al., Am. J. Physiol. 1989, 256:C799-C806) and causes a strong systemic lowering of the blood pressure, which is undesirable, since circulation in the ischemic regions may be reduced even further (“steal phenomenon”, L. C. Becker, Circulation 57, 1978, 1103-1110). Accordingly, high doses of adenosine are toxic, and the therapeutic value of systemically administered adenosine is limited.

Adenosine is a purine nucleoside and an intermediate of purine nucleotide degradation or purine nucleotide de novo synthesis. Adenosine kinase (ATP: adenosine 5′-phosphotransferase, EC 2.7.1.20) is one of the key enzymes in the regulation of the intercellular adenosine concentration (Arch and Newsholm, Essays Biochem. 1978, 14:82-123). Adenosine kinase is a ubiquitous enzyme which catalyses the phosphorylation of adenosine to AMP in the cytosol, where ATP is utilized as phosphate donor and Mg²⁺, presumably as MgATP²⁺ complex, is required for the reaction (Palella et al., J. Biol. Chem. 1980, 255:5264-5269). However, regulation of the adenosine concentration is also regulated depending on the respective metabolic situation of other enzymes, such as adenosine desaminase and S-adenosylhomocystein hydrolase.

The advantage of the adenosine kinase inhibition, compared to systemically administered adenosine, is the selectivity for ischemia. An adenosine kinase inhibitor can increase the concentration of adenosine, formed locally as a result of the ischemia, and in this manner—only in the ischemic region—effect maximum dilation of the blood vessels, improve perfusion and ensure that the cell-protective actions of adenosine are long-lasting. Thus, adenosine kinase inhibitors, administered orally or intravenously, can be employed for the prevention and/or treatment of ischemic disorders.

In addition, there are various indications of a neuroprotective, anticonvulsive, analgesic and sleep-inducing potential of adenosine kinase inhibitors, since they enhance the intrinsic effects of adenosine by inhibiting its cellular reuptake (K. A. Rudolphi et al., Cerebrovascular and Brain Metabolism Reviews 4, 1992, 364-369; T. F. Murray et al., Drug Dev. Res. 28, 1993, 410-415; T. Porkka-Heiskanen et al., Science 276, 1997, 1265-1268; “Adenosine in the Nervous System”, Ed.: Trevor Stone, Academic Press Ltd., 1991, 217-227; M. P. DeNinno, Annual Reports in Medicinal Chemistry 33, 1998, 111-120). Accordingly, adenosine kinase inhibitors can also be employed for the prophylaxis and treatment of acute and/or chronic pain (for a classification see “Classification of Chronic Pain, Descriptions of Chronic Pain Syndromes and Definitions of Pain Terms”, 2nd Ed., Meskey and Begduk, Ed,; IASP-Press, Seattle, 1994), neuropathic pain, such as, for example, that associated with diabetic neuropathy, post herpetic neuralgia, peripheral nerve damage, central pain and trigeminal neuralgia.

4-Aminofuro[2,3-d]pyrimidine derivatives having smooth-muscle-relaxing action are described in the Published Specification DE 1 817 146.

It is an object of the present invention to provide novel compounds having improved pharmaceutical properties. This object is achieved by the compounds of the formula (I) according to the invention which act as adenosine kinase inhibitors.

The present invention provides compounds of the formula (I)

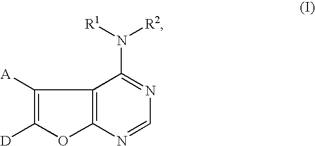

in which

A represents phenyl or 5- or 6-membered heteroaryl having up to three heteroatoms from the group consisting of N, O and/or S, each of which radicals may be substituted up to three times, independently of one another, by substituents from the group consisting of halogen, hydroxyl, (C₁-C₆)-alkoxy, trifluoromethyl, trifluoromethoxy, amino, carboxyl and (C₁-C₆)-alkoxycarbonyl,

or represents a group of the formula

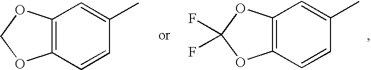

D represents a group of the formula

R³-E-G

in which

G represents phenylene or 5- or 6-membered heteroarylene having up to three heteroatoms from the group consisting of N, O and/or S, each of which radicals may be substituted up to two times, independently of one another, by substituents from the group consisting of halogen, trifluoromethyl, trifluoromethoxy, (C₁-C₆)-alkoxy, amino, nitro and carboxyl,

E represents a bond, a carbonyl group, a sulfonyl group or represents a group of the formula *—C(O)—NR⁴— or *—SO₂—NR⁴—,

in which * denotes the point of attachment to the group R³ and R⁴ represents hydrogen or (C₁-C₆)-alkyl,

and

R³ represents halogen, trifluoromethyl, hydroxyl, optionally hydroxyl- or amino-substituted (C₁-C₆)-alkoxy, trifluoromethoxy, nitro, carboxyl or a group of the formula H—C(O)—NR⁴—,

in which R⁴ is as defined above,

represents (C₁-C₆)-alkyl which may be mono- to disubstituted, independently of one another, by substituents selected from the group consisting of halogen, trifluoromethyl, hydroxyl (C₁-C₆)-alkoxy, amino, mono- or di-(C₁-C₆)-alkylamino, (C₁-C₆)-acylamino, (C₁-C₆)-alkoxycarbonylamino, amidino, guanidino, carboxyl, (C₁-C₆)-alkoxycarbonyl, (C₆-C₁₀)-aryl, (C₆-C₁₀)-aryloxy and 5- to 10-membered heteroaryl having up to three heteroatoms from the group consisting of N, O and/or S, where aryl, aryloxy and heteroaryl for their part may in each case be mono- to disubstituted, independently of one another, by halogen, hydroxyl, amino, (C₁-C₄)-alkyl, (C₁-C₆)-alkoxy, cyano or nitro,

represents (C₃-C₇)-cycloalkyl which may be substituted by phenyl or up to four times by (C₁-C₄)-alkyl,

represents (C₆-C₁₀)-aryl, which may be mono- to disubstituted, independently of one another, by substituents selected from the group consisting of halogen, trifluoromethyl, (C₁-C₆)-alkyl, hydroxyl, (C₁-C₆)-alkoxy, (C₁-C₆)-alkanoyl, cyano, nitro, amino, mono- and di-(C₁-C₆)-alkylamino, (C₁-C₆)-acylamino, carboxyl, (C₁-C₆)-alkoxycarbonyl and 5- to 6-membered heteroaryl having up to two heteroatoms from the group consisting of N, O and/or S,

represents 4- to 7-membered, saturated or partially unsaturated heterocyclyl which is attached via a ring carbon atom or via a ring nitrogen atom and has up to three heteroatoms from the group consisting of N, O and/or S, which may be substituted up to three times, independently of one another, by (C₁-C₆)-alkyl, which for its part may be substituted by hydroxyl, (C₁-C₄)-alkoxy or phenyl, (C₁-C₆)-alkoxycarbonyl, (C₁-C₆)-alkoxycarbonylamino, (C₁-C₆)-alkanoyl, (C₁-C₆)-alkylcarbonylamino, 1,2-dioxyethylene, carboxyl, amino, hydroxyl, (C₁-C₆)-alkoxy or an oxo group,

represents 5- to 10-membered heteroaryl which is attached via a ring carbon atom or via a ring nitrogen atom and has up to three heteroatoms from the group consisting of N, O and/or S, which for its part may optionally be mono- to disubstituted, independently of one another, by substituents selected from the group consisting of halogen, nitro, amino, hydroxyl (C₁-C₆)-alkyl, (C₃-C₆)-cycloalkyl, phenyl, benzyl and 5-membered heteroaryl having up to two heteroatoms from the group consisting of N, O and/or S,

or

represents a group of the formula —NR⁵R⁶,

in which

R⁵ and R⁶ independently of one another represent hydrogen, (C₁-C₆)-alkyl, which may be substituted by hydroxyl, amino, (C₁-C₄)-alkoxy, mono- or di-(C₁-C₄)-alkylamino or phenyl, represent (C₃-C₇)-cycloalkyl, which may be mono- to disubstituted, independently of one another, by hydroxyl, amino, (C₁-C₄)-alkoxy, mono- or di-(C₁-C₄)-alkylamino or (C₁-C₄)-alkyl, represent (C₆-C₁₀)-aryl, which may be mono- to disubstituted, independently of one another, by hydroxyl, halogen, amino, (C₁-C₄)-alkoxy or nitro, or represent 5- to 6-membered heterocyclyl or 5- to 6-membered heteroaryl having in each case up to two heteroatoms from the group consisting of N, O and/or S,

or

D represents a group of the formula

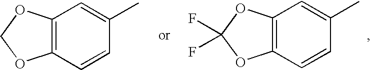

R¹ represents hydrogen, (C₃-C₆)-cycloalkyl or represents (C₁-C₆)-alkyl which may be mono- to disubstituted, independently of one another, by hydroxyl (C₁-C₆)-alkoxy, amino, mono- or di-(C₁-C₆)-alkylamino,

and

R² represents (C₁-C₆)-alkyl, which may be mono- to disubstituted, independently of one another, by substituents selected from the group consisting of trifluoromethyl, hydroxyl, (C₁-C₆)-alkoxy, amino, mono- or di-(C₁-C₆)-alkylamino, phenylamino, carboxyl, (C₁-C₆)-alkoxycarbonyl, (C₃-C₇)-cycloalkyl, phenyl, which for its part is optionally mono- to disubstituted by (C₁-C₄)-alkoxy, 5- to 6-membered heterocyclyl having up to two heteroatoms from the group consisting of N, O and/or S and 5- to 6-membered heteroaryl having up to two heteroatoms from the group consisting of N, O and/or S, which for its part is optionally substituted by (C₁-C₄)-alkyl or hydroxy-(C₁-C₄)-alkyl,

represents (C₆-C₁₀)-aryl which may be substituted by hydroxyl (C₁-C₆)-alkoxy, amino, mono or di-(C₁-C₆)-alkylamino,

represents 5- to 6-membered heteroaryl having up to three heteroatoms from the group consisting of N, O and/or S which may be substituted by (C₁-C₆)-alkyl, trifluoromethyl, halogen, hydroxyl, (C₁-C₆)-alkoxy, trifluoromethoxy, amino, mono- or di-(C₁-C₆)-alkylamino,

represents 5- to 6-membered heterocyclyl having up to two heteroatoms from the group consisting of N, O and/or S which may be substituted by benzyl or up to four times by (C₁-C₄)-alkyl,

or

represents (C₄-C₈)-cycloalkyl which may be mono- to disubstituted, independently of one another, by hydroxyl amino, (C₁-C₆)-alkyl, (C₁-C₆)-alkoxy, mono- or di-(C₁-C₆)-alkylamino or a group of the formula R⁷—C(O)—NH— or R⁷—SO₂—NH—,

in which

R⁷ represents (C₁-C₆)-alkoxy, phenyl, benzyl, phenylamino, mono- or di-(C₁-C₆)-alkylamino, which for their part are optionally substituted in the alkyl group by (C₁-C₄)-alkoxycarbonyl or carboxyl, or represents (C₄-C₇)-cycloalkylamino which for its part is optionally substituted in the cycloalkyl group by (C₁-C₄)-alkyl,

represents (C₁-C₆)-alkyl which may be substituted by hydroxyl, (C₁-C₆)-alkoxy, amino, mono or di-(C₁-C₆)-alkylamino, (C₁-C₆)-acylamino or by 5- or 6-membered heterocyclyl having up to two heteroatoms from the group consisting of N, O and/or S,

represents 5- or 6-membered heterocyclyl which is attached via a ring carbon atom or via a ring nitrogen atom and has up to two heteroatoms from the group consisting of N, O and/or S, which is optionally substituted by an oxo group,

or

represents 5- or 6-membered heteroaryl having up to three heteroatoms from the group consisting of N, O and/or S, which is optionally mono- to disubstituted by (C₁-C4)-alkyl,

or

R¹ and R² together with the nitrogen atom to which they are attached form a 4- to 11-membered mono-, bi- or spiro-cyclic heterocycle which may contain up to two further heteroatoms from the group consisting of N, O and/or S and which may be mono- to tetrasubstituted, independently of one another, by substituents selected from the group consisting of amino, mono- or di-(C₁-C₆)-alkylamino, hydroxyl (C₁-C₆)-alkoxy, oxo, carboxyl, carbamoyl, (C₁-C₆)-alkoxycarbonyl, (C₁-C₆)-alkoxycarbonylamino, (C₁-C₆)-alkanoyl, (C₁-C₆)-alkylcarbonylamino, (C₁-C₆)-alkyl, which for its part is optionally substituted by hydroxyl, (C₁-C₆)-alkoxy, amino, mono or di-(C₁-C₆)-alkylamino, phenyl or by 5- or 6-membered heterocyclyl having up to two heteroatoms from the group consisting of N, O and/or S, phenyl which for its part is optionally substituted by halogen, (C₃-C₇)-cycloalkyl, pyridyl, thienyl and 5- or 6-membered heterocyclyl having up to two heteroatoms from the group consisting of N, O and/or S,

and their pharmaceutically acceptable salts, solvates, hydrates and hydrates of the salts.

In the context of the invention, alkyl represents a straight-chain or branched alkyl radical having preferably 1 to 6, 1 to 4 or 1 to 2 carbon atoms. Preference is given to a straight-chain or branched alkyl radical having 1 to 4 carbon atoms. The following radicals may be mentioned by way of example and by way of preference: methyl, ethyl, n-propyl, isopropyl, n-, i-, s- or t-butyl, n-pentyl and n-hexyl.

In the context of the invention, aryl represents an aromatic radical having preferably 6 to 10 carbon atoms. Preferred aryl radicals are phenyl and naphthyl.

In the context of the invention, aryloxy represents an aromatic radical having preferably 6 to 10 carbon atoms which is attached via an oxygen atom. Preferred aryloxy radicals are phenoxy and napthoxy.

In the context of the invention, cycloalkyl represents a cycloalkyl group having preferably 3 to 8, 4 to 8, 3 to 7 or 3 to 5 carbon atoms. The following radicals may be mentioned by way of example and by way of preference: cyclopropyl, cyclobutyl, cyclopentyl, cyclohexyl and cycloheptyl.

In the context of the invention, alkoxy preferably represents a straight-chain or branched alkoxy radical having 1 to 6, 1 to 4 or 1 to 2 carbon atoms. Preference is given to a straight-chain or branched alkoxy radical having 1 to 2 carbon atoms. The following radicals may be mentioned by way of example and by way of preference: methoxy, ethoxy, n-propoxy, isopropoxy, t-butoxy, n-pentoxy and n-hexoxy.

In the context of the invention, alkoxycarbonyl preferably represents a straight-chain or branched alkoxy radical having from 1 to 6 or 1 to 4 carbon atoms which is attached via a carbonyl group. Preference is given to a straight-chain or branched alkoxycarbonyl radical having 1 to 4 carbon atoms. The following radicals may be mentioned by way of example and by way of preference: methoxycarbonyl, ethoxycarbonyl, n-propoxycarbonyl, isopropoxycarbonyl and t-butoxycarbonyl.

In the context of the invention, alkanoyl preferably represents a straight-chain or branched alkyl radical having 1 to 6 or 1 to 4 carbon atoms which carries a doubly attached oxygen atom in the 1-position and is attached via the 1-position. Preference is given to a straight-chain or branched alkanoyl radical having 1 to 4 carbon atoms. The following radicals may be mentioned by way of example and by way of preference: formyl, acetyl, propionyl, n-butyryl, i-butyryl, pivaloyl and n-hexanoyl.

In the context of the invention, alkonoyloxy preferably represents a straight-chain or branched alkyl radical having from 1 to 6, 1 to 4 or 1 to 2 carbon atoms which carries a doubly attached oxygen atom in the 1-position and is attached in the 1-position via a further oxygen atom. Preference is given to a straight-chain or branched alkanoyloxy radical having 1to 2 carbon atoms. The following radicals may be mentioned by way of example and by way of preference: acetoxy, propionoxy, n-butyroxy, i-butyroxy, pivaloyloxy and n-hexanoyloxy.

In the context of the invention, monoalkylamino represents an amino group having a straight-chain or branched alkyl substituents which preferably has 1 to 6, 1 to 4 or 1 to 2 carbon atoms. Preference is given to a straight-chain or branched monoalkylamino radical having 1 to 4 carbon atoms. The following radicals may be mentioned by way of example and by way of preference: methylamino, ethylamino, n-propylamino, isopropylamino, t-butylamino, n-pentylamino and n-hexylamino.

In the context of the invention, dialkylamino represents an amino group having two identical or different straight-chain or branched alkyl substituents each preferably having 1 to 6, 1 to 4 or 1 to 2 carbon atoms. Preference to straight-chain or branched dialkylamino radicals each having 1 to 4 carbon atoms. The following radicals may be mentioned by way of example and by. way of preference: N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

In the context of the invention, cycloalkylamino represents an amino group having a cycloalkyl substituent which preferably has 4 to 7, 4 to 6 or 5 to 6 carbon atoms. The following radicals may be mentioned by way of example and by way of preference: cyclobutylamino, cyclopentylamino, cyclohexylamino and cyloheptylamino.

In the context of the invention, acylamino represents an amino group having a straight-chain or branched alkanoyl substituent which preferably has 1 to 6, 1 to 4 or 1 to 2 carbon atoms and is attached via the carbonyl group. Preference is given to an acylamino radical having 1 to 2 carbon atoms. The following radicals may be mentioned by way of example and by way of preference: formamido, acetamido, propionamido, n-butyramido and pivaloylamido.

In the context of the invention, alkylcarbonylamino represents an amino group having a straight-chain or branched alkylcarbonyl substituent (=alkanoyl substituent) which preferably has 1 to 6 or 1 to 4 carbon atoms in the alkyl radical and is attached via the carbonyl group. Preference is given to an alkylcarbonylamino radical having 1 to 4 carbon atoms. The following radicals may be mentioned by way of example and by way of preference: methylcarbonylamino, ethylcarbonylamino, n-propylcarbonylamino and t-butylcarbonylamino.

In the context of the invention, alkoxycarbonylamino represents an amino group having a straight-chain or branched alkoxycarbonyl substituent which preferably has 1 to 6 or 1 to 4 carbon atoms in the alkoxy radical and is attached via the carbonyl group. Preference is given to alkoxycarbonylamino radical having 1 to 4 carbon atoms. The following radicals may be mentioned by way of example and by way of preference: methoxycarbonylamino, ethoxycarbonylamino, n-propoxycarbonylamino and t-butoxycarbonylamino.

In the context of the invention, 5- to 6-membered or 5- to 10-membered heteroaryl having up to 3 identical or different heteroatoms from the group consisting of N, O and/or S preferably represents a mono- or bicyclic aromatic heterocycle which is attached via a ring carbon atom of the heteroaromatic ring or, if appropriate, via a nitrogen ring atom of the heteroaromatic ring. Examples which may be mentioned are: furyl, pyrrolyl, thienyl, thiazolyl, oxazolyl, imidazolyl, triazolyl, pyridyl, pyrimidinyl, pyriadazinyl. Preference is given to pyridyl, pyrimidinyl, pyridazinyl, furyl and thiazolyl.

In the context of the invention, a 4- to 7-membered saturated or partially unsaturated heterocycle having up to three identical or different heteroatoms from the group consisting of N, O and/or S preferably represents a non aromatic heterocycle which may contain one or, if appropriate, two double bonds and which is attached via a ring carbon atom or, if appropriate, via a ring nitrogen atom. Preference is given to a 5- to 6-membered saturated heterocycle having up to two identical or different heteroatoms from the group consisting of N, O and/or S. Examples which may be mentioned are: tetrahydrofur-2-yl, tetrahydrofur-3-yl, pyrrolidine-1-yl, pyrrolidine-2-yl, pyrrolidine-3-yl, pyrroline-1-yl, piperidine-1-yl, piperidine-4-yl, 1,2,dihydropyridine-1-yl, 1,4-dihydropyridine-1-yl, piperazine-1-yl, morpholine-4-yl, thiomorpholine-4-yl. Preference is given to piperidinyl, piperazinyl, morpholinyl and pyrrolidinyl.

In the context of the invention, halogen includes fluorine, chlorine, bromine and iodine. Preference is given to fluorine, chlorine or bromine.

Depending on the substitution pattern, the compounds according to the invention can exist in stereoisomeric forms which are either like image and mirror image (enantiomers) or which are not like image and mirror image (diastereomers). The invention relates both to the enantiomers or diastereomers and to their respective mixtures. The racemic forms, like the diastereomers, can be separated in a known manner into the stereoisomerically uniform components.

Furthermore, certain compounds can be present in tautomeric forms. This is known to the person skilled in the art, and such compounds are likewise included in the scope of the invention.

The compounds according to the invention can also be present as salts. In the context of the invention, preference is given to physiologically acceptable salts.

Physiologically acceptable salts can be salts of the compounds according to the invention with inorganic or organic acids. Preference is given to salts with inorganic acids, such as, for example, hydrochloric acid, hydrobromic acid, phosphoric acid or sulfuric acid, or salts with organic carboxylic or sulfonic acids, such as, for example, acetic acid, propanoic acid, maleic acid, fumaric acid, malic acid, citric acid, tartaric acid, lactic acid, benzoic acid, or methanesulfonic acid, ethanesulfonic acid, benzenesulfonic acid, toluenesulfonic acid or naphthalenedisulfonic acid.

Physiological acceptable salts can also be salts of the compounds according to the invention with bases, such as, for example, metal or ammonium salts. Preferred examples are alkaline metal salts (for example sodium or potassium salts), alkaline earth metal salts (for example magnesium or calcium salts), and also ammonium salts which are derived from ammonia or organic amines, such as, for example, ethylamine, di- or triethylamine, ethyldiisopropylamine, monoethanolamine, di- or triethanolamine, dicyclohexylamine, dimethylaminoethanol, dibenzylamine, N-methylmorpholine, dihydroabietylamine, 1-ephenamine, methylpiperidine, arginine, lysine, ethylenediamine or 2-phenylethylamine.

The compounds according to the invention can also be present in the form of their solvates, in particular in the form of their hydrates.

Moreover, the invention also embraces prodrugs of the compounds according to the invention. According to the invention, “prodrugs” are those derivatives of the compounds of the general formula (I) which for their part may be biologically less active or even inactive, but which, following application, are, under physiological conditions, converted into the corresponding biologically active form (for example metabolically, solvolytically or in another way).

Preference is given to compounds of the formula (I) in which

A represents phenyl which may be substituted by fluorine, chlorine, bromine or methoxy,

and

G represents 1,3- or 1,4-phenylene which may be mono- or disubstituted by methoxy, or represents a group of the formula

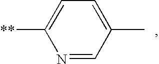

in which ** denotes the point of attachment to the group E.

Preference is likewise given to compounds of the, formula (I) in which

E represents a bond, represents a carbonyl group or represents a group of the formula *—C(O)—NH—,

in which * denotes the point of attachment to the group R³

Preference is likewise given to compounds of the formula (I) in which

R¹ represents hydrogen, methyl or ethyl, which may be substituted by hydroxyl,

and

R² represents (C₁-C₄)-alkyl which may be mono- or disubstituted, independently of one another, by hydroxyl, amino, (C₁-C₄)-alkoxy, mono- or di-(C₁-C₄)-alkylamino, or represents (C₅-C₇)-cycloalkyl,

or

R¹ and R² together with the nitrogen atom to which they are attached form a pyrrolidine, piperidine, piperazine or morpholine ring which may be mono-or disubstituted, independently of one another, by (C₁-C₄)-alkyl, which for its part is optionally substituted by hydroxyl or amino, by amino, mono- or di-(C₁-C₄)-alkylamino, hydroxyl (C₁-C₄)-alkoxy, oxo, carboxyl, carbamoyl or by (C₁-C₄)-alkoxycarbonyl.

Particular preference is given to compounds of the general formula (Ia)

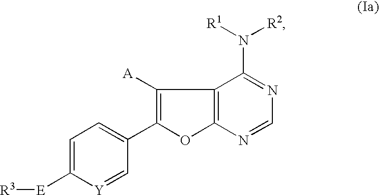

in which

A represents phenyl which may be substituted by fluorine, chlorine, bromine or methoxy,

Y represents CH or N,

E represents a bond, represents a carbonyl group or represents a group of the formula *—C(O)—NH—,

in which * denotes the point of attachment to the group R³,

R³ represents hydroxyl, methoxy, amino or mono-(C₁-C₄)-alkylamino, which may be substituted in the alkyl group by hydroxyl or amino,

represents (C₁-C₄)-alkyl which is optionally substituted by hydroxyl methoxy, ethoxy, amino, mono or dimethylamino, (C₁-C₄)-alkoxycarbonylamino, acetamido, carboxyl or (C₁-C₄)-alkoxycarbonyl,

or

represents a 4- to 6-membered saturated heterocycle which is attached via a ring carbon atom or via a ring nitrogen atom and has up to two heteroatoms from the group consisting of N and/or O, which may be substituted up to two times, independently of one another, by (C₁-C₄)-alkyl, which for its part may be substituted by hydroxyl methoxy or ethoxy, by carboxyl, amino, hydroxyl methoxy, ethoxy or an oxo group,

R¹ represents hydrogen, methyl or represents ethyl which may be substituted by hydroxyl or amino,

and

R² represents (C₁-C₄)-alkyl which may be mono- to disubstituted, independently of one another, by hydroxyl, amino, (C₁-C₄)-alkoxy, mono- or di-(C₁-C₄)-alkylamino, or represents (C₅-C₇)-cycloalkyl

or

R¹ and R² together with the nitrogen atom to which they are attached form a pyrrolidine, piperidine, piperazine, or morpholine ring which may be mono-or disubstituted, independently of one another, by (C₁-C₄)-alkyl, which for its part is optionally substituted by hydroxyl or amino, by amino, mono or di-(C₁-C₄)-alkylamino, hydroxyl, (C₁-C₄)-alkoxy, oxo, carboxyl, carbamoyl or by (C₁-C₄)-alkoxycarbonyl.

and their pharmaceutically acceptable salts, solvates, hydrates and hydrates of the salts.

Very particular preference is given to compounds of the general formula (Ia)

in which

A represents phenyl which can be substituted by fluorine,

Y represents CH or N,

E represents a bond or represents a group of the formula *—C(O)—NH—,

in which * denotes the point of attachment to the group R³,

R³ represents amino or mono-(C₁-C₄)-alkylamino which may be substituted in the alkyl group by hydroxyl or amino,

represents (C₁-C₄)-alkyl which is optionally substituted by hydroxyl, amino, mono- or dimethylamino,

or

represents pyrrolidine, piperazine or piperidine which may be substituted up to two times, independently of one another, by methyl, ethyl, n-propyl, isopropyl, n-butyl, sec-butyl, isobutyl or tert-butyl, which for their part may be substituted by hydroxyl, by amino or hydroxyl,

R¹ and R² together with the nitrogen atom to which they are attached form a piperidine, piperazine or morpholine ring which may be mono- or disubstituted, independently of one another, by methyl, ethyl, n-propyl or isopropyl, which for their part are optionally substituted by hydroxyl or amino, by amino, hydroxyl or oxo,

and their pharmaceutically acceptable salts, solvates, hydrates and hydrates of the salts.

The general or preferred radical definitions given above apply both to the end products of the formula (I) and, correspondingly, to the starting materials or intermediates required in each case for the preparations.

The individual radical definitions given in the respective combinations or preferred combinations of radicals are, independently of the combinations of radicals given in each case, also replaced by radical definitions of other combinations.

It has now been found, that compounds of the general formula (I)

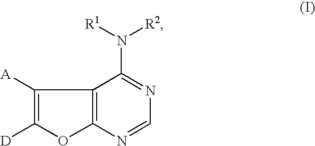

in which

A represents phenyl or 5- or 6-membered heteroaryl having up to three heteroatoms from the group consisting of N, O and/or S, each of which radicals may be substituted up to three times, independently of one another, by substituents from the group consisting of halogen, hydroxyl, (C₁-C₆)-alkoxy, trifluoromethyl, trifluoromethoxy, amino, carboxyl and (C₁-C₆)-alkoxycarbonyl,

or represents a group of the formula

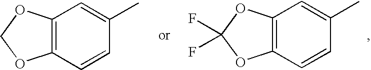

D represents a group of the formula

R³-E-G

in which

G represents phenylene or 5- or 6-membered heteroarylene having up to three heteroatoms from the group consisting of N, O and/or S, each of which radicals may be substituted up to two times, independently of one another, by substituents from the group consisting of halogen, trifluoromethyl, trifluoromethoxy, (C₁-C₆)-alkoxy, amino, nitro and carboxyl,

E represents a bond, a carbonyl group, a sulfonyl group or represents a group of the formula *—C(O)—NR⁴— or *—SO₂—NR⁴—,

in which * denotes the point of attachment to the group R³ and R⁴ represents hydrogen or (C₁-C₆)-alkyl,

and

R³ represents halogen, trifluoromethyl, hydroxyl, (C₁-C₆)-alkoxy, trifluoromethoxy, nitro, carboxyl or a group of the formula H—C(O)—NR⁴—,

in which R⁴ is as defined above,

represents (C₁-C₆)-alkyl which may be mono- to disubstituted, independently of one another, by substituents selected from the group consisting of halogen, trifluoromethyl, hydroxyl, (C₁-C₆)-alkoxy, amino, mono- or di-(C₁-C₆)-alkylamino, (C₁-C₆)-acylamino, (C₁-C₆)-alkoxycarbonylamino, amidino, guanidino, carboxyl, (C₁-C₆)-alkoxycarbonyl, (C₆-C₁₀)-aryl, (C₆-C₁₀)-aryloxy and 5- to 10-membered heteroaryl having up to three heteroatoms from the group consisting of N, O and/or S, where aryl, aryloxy and heteroaryl for their part may in each case be mono- to disubstituted, independently of one another, by halogen, hydroxyl, amino, (C₁-C₄)-alkyl, (C₁-C₆)-alkoxy, cyano or nitro,

represents (C₃-C₇)-cycloalkyl which may be substituted by phenyl or up to four times by (C₁-C₄)-alkyl,

represents (C₆-C₁₀)-aryl, which may be mono- to disubstituted, independently of one another, by substituents selected from the group consisting of halogen, trifluoromethyl, (C₁-C₆)-alkyl, hydroxyl, (C₁-C₆)-alkoxy, (C₁-C₆)-alkanoyl, cyano, nitro, amino, mono- and di-(C₁-C₆)-alkylamino, (C₁-C₆)-acylamino, carboxyl, (C₁-C₆)-alkoxycarbonyl and 5- to 6-membered heteroaryl having up to two heteroatoms from the group consisting of N, O and/or S,

represents 4- to 7-membered, saturated or partially unsaturated heterocyclyl which is attached via a ring carbon atom or via a ring nitrogen atom and has up to three heteroatoms from the group consisting of N, O and/or S, which may be substituted up to three times, independently of one another, by (C₁-C₆)-alkyl, which for its part may be substituted by hydroxyl, (C₁-C₄)-alkoxy or phenyl, (C₁-C₆)-alkoxycarbonyl, carboxyl, amino, hydroxyl, (C₁-C₆)-alkoxy or an oxo group,

represents 5- to 10-membered heteroaryl which is attached via a ring carbon atom or via a ring nitrogen atom and has up to three heteroatoms from the group consisting of N, O and/or S, which for its part may optionally be mono- to disubstituted, independently of one another, by substituents selected from the group consisting of halogen, nitro, amino, hydroxyl (C₁-C₆)-alkyl, (C₃-C₆)-cycloalkyl, phenyl, benzyl and 5-membered heteroaryl having up to two heteroatoms from the group consisting of N, O and/or S,

or

represents a group of the formula —NR⁵R⁶,

in which

R⁵ and R⁶ independently of one another represent hydrogen, (C₁-C₆)-alkyl, which may be substituted by hydroxyl, amino, (C₁-C₄)-alkoxy, mono- or di-(C₁-C₄)-alkylamino or phenyl, represent (C₃-C₇)-cycloalkyl, which may be mono- to disubstituted, independently of one another, by hydroxyl, amino, (C₁-C₄)-alkoxy, mono- or di-(C₁-C₄)-alkylamino or (C₁-C₄)-alkyl, represent (C₆-C₁₀)-aryl, which may be mono- to disubstituted, independently of one another, by hydroxyl, amino, (C₁-C₄)-alkoxy or nitro, or represent 5- to 6-membered heterocyclyl or 5- to 6-membered heteroaryl having in each case up to two heteroatoms from the group consisting of N, O and/or S,

or

D represents a group of the formula

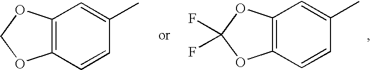

R¹ represents hydrogen, (C₃-C₆)-cycloalkyl or represents (C₁-C₆)-alkyl which may be mono- to disubstituted, independently of one another, by hydroxyl (C₁-C₆)-alkoxy, amino, mono- or di-(C₁-C₆)-alkylamino,

and

R² represents (C₁-C₆)-alkyl, which may be mono- to disubstituted, independently of one another, by substituents selected from the group consisting of trifluoromethyl, hydroxyl, (C₁-C₆)-alkoxy, amino, mono- or di-(C₁-C₆)-alkylamino, phenylamino, carboxyl, (C₁-C₆)-alkoxycarbonyl, (C₃-C₇)-cycloalkyl, phenyl, which for its part is optionally mono- to disubstituted by (C₁-C₄)-alkoxy, 5- to 6-membered heterocyclyl having up to two heteroatoms from the group consisting of N, O and/or S and 5- to 6-membered heteroaryl having up to two heteroatoms from the group consisting of N, O and/or S, which for its part is optionally substituted by (C₁-C₄)-alkyl or hydroxy-(C₁-C₄)-alkyl,

represents (C₆-C₁₀)-aryl which may be substituted by hydroxyl (C₁-C₆)-alkoxy, amino, mono or di-(C₁-C₆)-alkylamino,

represents 5- to 6-membered heteroaryl having up to three heteroatoms from the group consisting of N, O and/or S which may be substituted by (C₁-C₆)-alkyl, trifluoromethyl, halogen, hydroxyl, (C₁-C₆)-alkoxy, trifluoromethoxy, amino, mono- or di-(C₁-C₆)-alkyl amino,

represents 5- to 6-membered heterocyclyl having up to two heteroatoms from the group consisting of N, O and/or S which may be substituted by benzyl or up to four times by (C₁-C₄)-alkyl,

or

represents (C₄-C₈)-cycloalkyl which may be mono- to disubstituted, independently of one another, by hydroxyl amino, (C₁-C₆)-alkyl, (C₁-C₆)-alkoxy, mono- or di-(C₁-C₆)-alkylamino or a group of the formula R⁷—C(O)—NH— or R⁷—SO₂—NH—,

in which

R⁷ represents (C₁-C₆)-alkoxy, phenyl, benzyl, phenylamino, mono- or di-(C₁-C₆)-alkylamino, which for their part are optionally substituted in the alkyl group by (C₁-C₄)-alkoxycarbonyl or carboxyl, or represents (C₄-C₇)-cycloalkylamino which for its part is optionally substituted in the cycloalkyl group by (C₁-C₄)-alkyl,

represents (C₁-C₆)-alkyl which may be substituted by hydroxyl, (C₁-C₆)-alkoxy, amino, mono or di-(C₁-C₆)-alkylamino, (C₁-C₆)-acylamino or by 5- or 6-membered heterocyclyl having up to two heteroatoms from the group consisting of N, O and/or S,

represents 5- or 6-membered heterocyclyl which is attached via a ring carbon atom or via a ring nitrogen atom and has up to two heteroatoms from the group consisting of N, O and/or S, which is optionally substituted by an oxo group,

or

represents 5- or 6-membered heteroaryl having up to three heteroatoms from the group consisting of N, O and/or S, which is optionally mono- to disubstituted by (C₁-C₄)-alkyl,

or

R¹ and R² together with the nitrogen atom to which they are attached form a 4- to 11-membered mono-, bi- or spiro-cyclic heterocycle which may contain up to two further heteroatoms from the group consisting of N, O and/or S and which may be mono- to tetrasubstituted, independently of one another, by substituents selected from the group consisting of amino, mono- or di-(C₁-C₆)-alkylamino, hydroxyl (C₁-C₆)-alkoxy,. oxo, carboxyl, carbamoyl, (C₁-C₆)-alkoxycarbonyl, (C₁-C₆)-alkyl, which for its part is optionally substituted by hydroxyl, (C₁-C₆)-alkoxy, amino, mono or di-(C₁-C₆)-alkylamino, or by 5- or 6-membered heterocyclyl having up to two heteroatoms from the group consisting of N, O and/or S, phenyl which for its part is optionally substituted by halogen, (C₃-C₇)-cycloalkyl, pyridyl and 5- or 6-membered heterocyclyl having up to two heteroatoms from the group consisting of N, O and/or S,

and their pharmaceutically acceptable salts, solvates, hydrates and hydrates of the salts,

are pharmacologically active and can be used as pharmaceuticals or for preparing formulations of pharmaceuticals.

The present invention also provides a process for preparing the compounds of the formula (I), characterized in that

compounds of the formula (II)

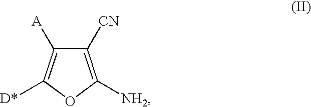

in which D* represents D or an unsubstituted phenyl ring and A and D are as defined above,

are either

[A] initially reacted with formic acid in acetic anhydride to give compounds of the formula (III)

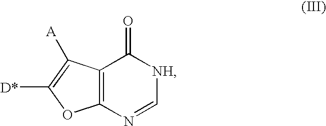

in which D* represents D or represents an unsubstituted phenyl ring and A and D are as defined in as defined above,

then converted with phosphoryl chloride into compounds of the formula (IV)

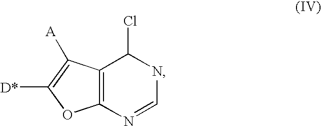

in which D* represents D or represents an unsubstituted phenyl ring and A and D are as defined above,

if D* represents an unsubstituted phenyl ring, this phenyl ring is then nitrated, and are finally reacted with compounds of the formula (V)

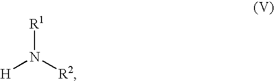

in which R¹ and R² are as defined above,

to compounds of the formula (I)

or

[B] initially converted with triethyl orthoformate into compounds of the formula (VI)

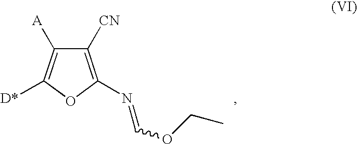

in which D* represents D or represents an unsubstituted phenyl ring and A and D are as defined above,

and then reacted with compounds of the formula (VII)

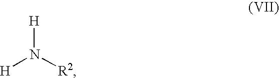

in which R² is as defined above,

and then reacted directly or, if D* represents an unsubstituted phenyl ring, with a base by subsequent nitration of this phenyl ring, to give compounds of the formula (I),

where the resulting compounds of the formula (I) can, if appropriate, subsequently be subjected to further derivatizations which can be carried out by customary methods.

The process according to the invention can be illustrated in an exemplary manner by the formula schemes below:

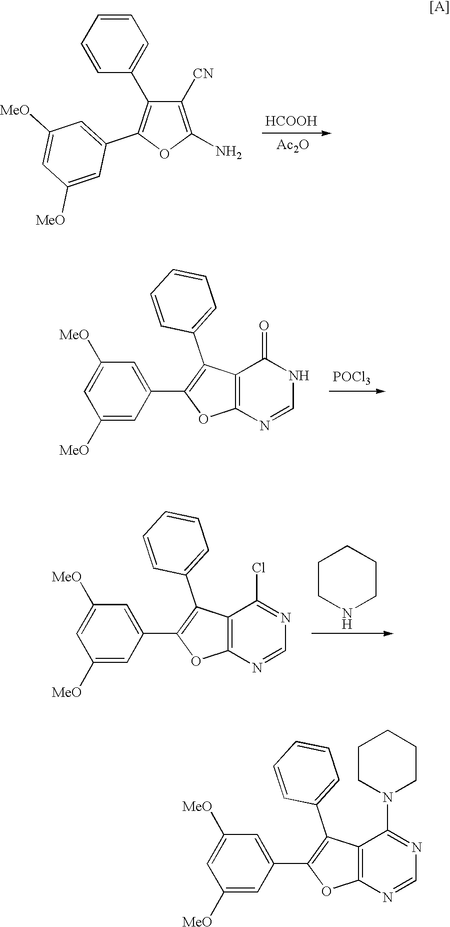

if D* represents an unsaturated phenyl ring:

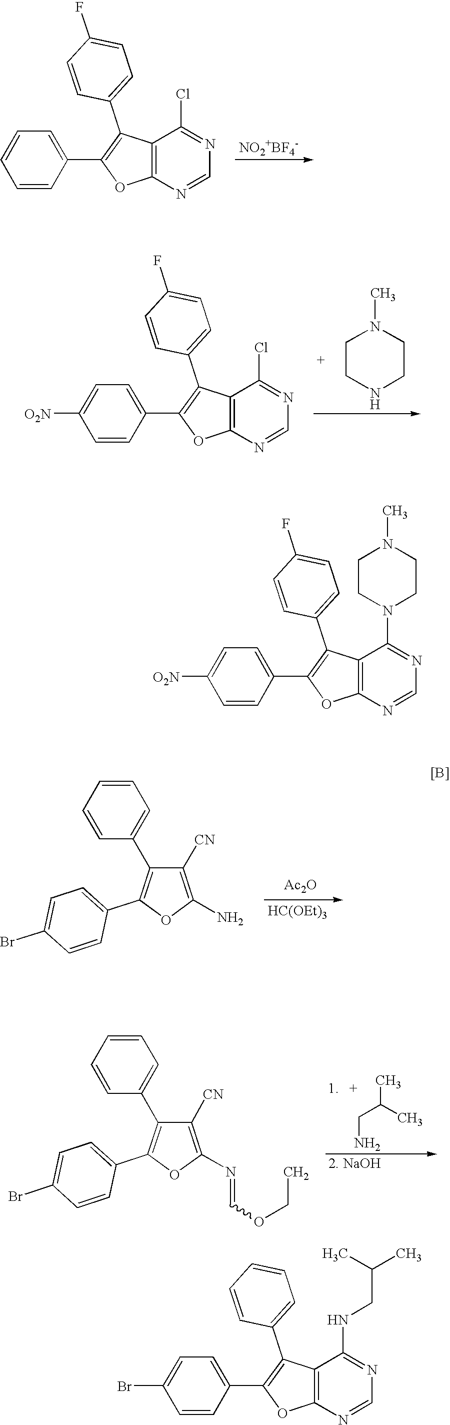

if D* represents an unsaturated phenyl ring:

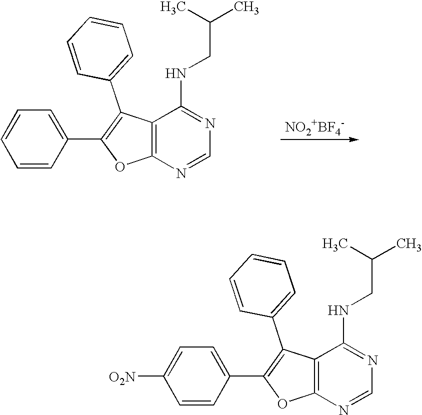

Here, suitable solvents for the process described above are organic solvents which are inert under the reaction conditions, or water. These include halogenated hydrocarbons, such as dichloromethane, trichloromethane, carbon tetrachloride, 1,2-dichloroethane, trichloroethane, tetrachloroethane, 1,2, dichloroethylene or trichloroethylene, ethers, such as diethyl ether, dioxane, tetrahydrofuran, glycol dimethyl ether or diethylene glycol dimethyl ether, alcohols, such as methanol, ethanol, propanol, isopropanol or butanol, hydrocarbons, such as benzene, xylene, toluene, hexane or cyclohexane, or other solvents, such as dimethylforamide, dimethyl sulfoxide, N-methylpyrrolidone, acetonitrile or pyridine, or mixtures thereof.

The reactions are generally carried out in a temperature range of from −78° C. to 150° C.

The reactions can be carried out under atmospheric, elevated or reduced pressure (for example in the range from 0.5 to 5 bar). In general, the reactions are carried out under atmospheric pressure.

Suitable bases are the customary inorganic or organic bases. These preferably include alkaline metal and alkaline earth metal hydroxides, such as, for example, lithium, sodium hydroxide or potassium hydroxide, or alkaline metal and alkaline earth metal carbonates, such as sodium carbonate or potassium carbonate, or sodium methoxide or potassium methoxide or sodium ethoxide or potassium ethoxide or potassium tert-butoxide, or amides, such as sodium amide, lithium bis-(trimethylsilyl)amide or lithium diisopropylamide, or amines, such as triethylamine, diisopropylethylamine, diisopropylamine, N-methylmorpholine, 4-dimethylaminopyridine or pyridine.

Reaction step [A] (II)→(III) is preferably carried out in a mixture of formic acid and acetic anhydride in a ratio of 2:1 (v:v) as solvent. The temperature range for this reaction is preferably between 50° C. and 100° C., in particular at reflux temperature.

The reaction step [A] (III)→(IV) is preferably carried out in an excess of phosphoryl chloride, for example 20- to 50-fold, as solvent. The temperature range for this reaction is preferably between 80° C. and 120° C., in particular at reflux temperature.

The reaction step [A] (IV)+(V)→(I) is preferably carried out in the absence of a solvent or using the solvent ethanol and an excess, for example 4-fold, of the amine (V). The temperature range for this reaction is preferably between 60° C. and 90° C., in particular at reflux temperature.

If D* represents an unsubstituted phenyl ring, the nitration of the phenyl ring in compounds of the formula (IV) or in the reaction products of the reaction (VI)+(VIII) is preferably carried out in acetonitrile or dichloromethane as solvent. The temperature range for this reaction is preferably between 0° C. and 30° C., in particular at 5° C. The preferred nitrating agent is nitronium tetrafluoroborate.

The reaction step [B] (II)→(VI) is preferably carried out in an excess of ethyl orthoformate as solvent in the presence of acetic anhydride as condensing agent. The temperature range for this reaction is preferably between 100° C. and 150° C., in particular at reflux temperature.

The reaction step [B] (VI)+(VII)→(I) is preferably carried out in the absence of a solvent or using the solvent ethanol and an excess, for example 4-fold, of the amine (V). The temperature range of this reaction is preferably between 20° C. and 60° C., in particular at 40° C. The base used in the second part-step of this reaction is preferably aqueous sodium hydroxide solution, at a temperature of preferably between 60° C. and 120° C., in particular at 90° C.

Some of the compounds of the formula (II) are known from the literature and can be prepared, for example,

by converting compounds of the formula (VIII)

A-CHO (VIII),

in which

A is as defined above

with triethyl phosphite and chlorotrimethylsilane into compounds of the formula (IX)

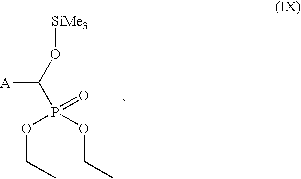

in which

A is as defined above,

followed by reaction, in the presence of a base, with compounds of the formula (X)

D*-CHO (X)

in which

D* represents D or represents an unsubstituted phenyl ring and D is as defined above,

giving compounds of the formula (XI)

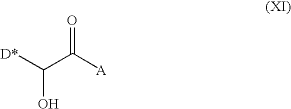

in which D* represents D or represents an unsubstituted phenyl ring and A and D are as defined above,

which are then converted in the presence of a base with malononitrile into compounds of the formula (II).

The reaction sequence is illustrated by the reaction scheme below:

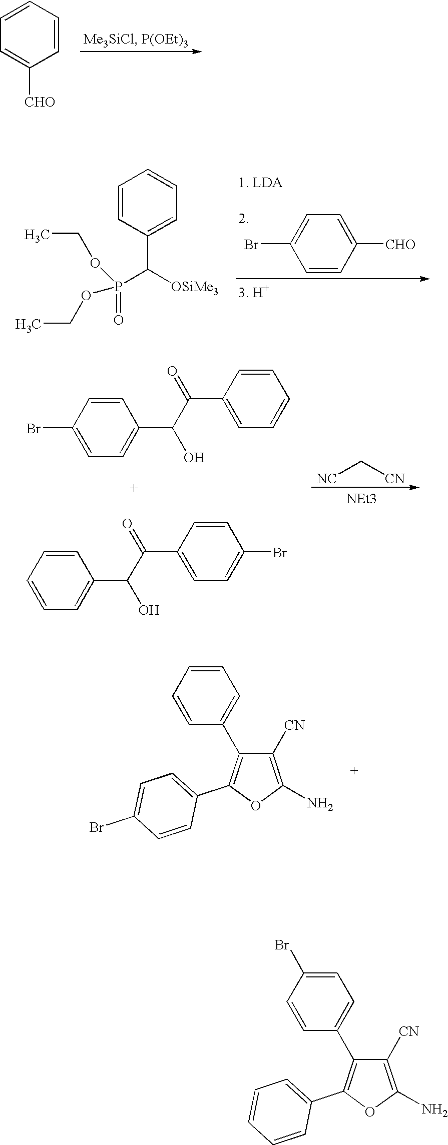

The compounds of the formula (XI) are generally obtained as a mixture of isomers. Depending on the substituents, the isomers formed can also be separated at various further stages of the synthesis, since it is also possible to use the isomer mixtures for the subsequent reactions.

The reaction step (VIII)→(IX) is preferably carried out neat, without further solvent. The temperature range for this reaction is preferably between 60° C. and 120° C., in particular at 80° C.

The reaction step (IX)+(X)→(XI) is preferably carried out in tetrahydrofuran as solvent. The temperature range for this reaction is preferably between −78° C. and room temperature, in particular at −70° C. The base used is preferably lithium diisopropylamide.

The reaction step (XI)→(II) is preferably carried out in dimethylformamide as solvent. The temperature range for this reaction is preferably between 0° C. and 40° C., in particular at room temperature. The base used is preferably triethylamine.

The compounds of the formulae (V), (VII), (VIII) and (X) are commercially available, known from the literature or preparable by customary methods.

The compounds of the formula (I) have a surprising and useful pharmacological activity spectrum and can therefore be used as versatile medicaments.

The pharmaceutical activity of the compounds of the formula (I) can be explained by their action as adenosine kinase inhibitors.

The compounds of the formula (I), alone or in combination with one or more other active compounds, are suitable for the prevention and/or treatment in particular of ischemia-related peripheral and cardiovascular disorders. Suitable active compounds for combinations are in particular pharmaceuticals which are part of the standard therapy of coronary heart disease, such as, for example, calcium canal blockers, nitro vasodilators, beta receptor blockers, platelet aggregation inhibitors, thrombolytics (fibrinolytics), anticoagulants, ACE inhibitors, glycol protein IIb/IIIa receptor antagonists, anti-arrhythmics, beta-adrenergic agonists or nucleoside transporter inhibitors.

In the context of the present invention, ischemia-related peripheral and cardiovascular disorders are to be understood as meaning, for example and in particular, acute and chronic treatment of ischemic disorders of the cardiovascular system, such as, for example, coronary heart disease, stable and unstable angina pectoris, peripheral and arterial occlusion diseases, thrombotic vessel occlusions, myocardial infarction and reperfusion damage.

In addition, the compounds of the formula (I) can be used, for example and in particular, for the prevention and/or treatment of cerebral ischemia, stroke, reperfusion damage, brain trauma, odema, spasms, epilepsy, respiratory arrest, cardiac arrest, Reye syndrome, cerebral thrombosis, embolism, tumors, hemorrhages, encephalomyelitis, hydroencephalitis, spinal injuries, post-operative brain damage, injuries of the retina or the optical nerve following glaucoma, ischemia, hypoxia, edema or trauma and in the treatment of schizophrenia, sleep disorders and acute and/or chronic pain and also neurodegenerative disorders, in particular for the prevention and/or treatment of cancer-induced pain and chronic neuropathic pain, such as, for example, that associated with diabetic neuropathy, post therapeutic neuralgia, peripheral nerve damage, central pain (for example as a result of cerebral ischemia) and trigeminal neuralgia and other chronic pain, such as, for example, lumbago, lower back pain or rheumatic pain.

The compounds of the formula (I) are furthermore suitable, for example and in particular, for the prevention and/or treatment of high blood pressure and cardiac insufficiency, myocarditis, nephritis, pancreatitis and diabetic nephropathy.

The present invention also relates to the use of the compounds of the formula (I) for preparing medicaments for the prophylaxis and/or treatment of the syndromes mentioned above.

The present invention furthermore relates to a method for the prophylaxis and/or treatment of symptoms mentioned above using the compounds of the formula (I).

The present invention further provides pharmaceuticals comprising at least one compound of the formula (I), preferably together with one or more pharmaceutically acceptable auxiliaries or carriers, and their use for the purposes mentioned above.

All conventional modes of application are suitable for administering the compounds of the general formula (I), that is to say oral, parenteral, inhalation, nasal, sublingual, buccal, rectal, local, such as, for example, in the case of implants or stents, or external, such as, for example, transdermal, particularly preferably oral or parenteral. For parenteral administration, mention must be made in particular of intravenous, intramuscular and subcutaneous administration, for example as subcutaneous depot. Oral or parenteral administration is preferred. Oral administration is particularly preferred.

In this connection, the active compounds can be administered alone or in the form of preparations. Preparations suitable for oral administration are, inter alia, tablets, capsules, pellets, coated tablets, pills, granules, solid and liquid aerosols, syrups, emulsions, suspensions and solutions. The active compound must be present therein in an amount such that a therapeutic effect is achieved. The active compound can be present in general in a concentration of from 0.1 to 100% by weight, in particular 0.5 to 90% by weight, preferably 5to 80% by weight. The concentration of active compound ought in particular to be 0.5 to 90% by weight, i.e. the active compound should be present in amounts sufficient to achieve the indicated dosage range.

For this purpose, the active compounds can be converted in a manner known per se into conventional preparations. This takes place by using inert, non-toxic, pharmaceutically acceptable carriers, auxiliaries, solvents, vehicles, emulsifiers and/or dispersants.

Excipients which may be mentioned, for example, are: water, non-toxic organic solvents, such as, for example, paraffins, vegetable oils (for example sesame oil), alcohols (for example ethanol, glycerol), glycols (for example polyethylene glycol), solid carriers, such as natural or synthetic ground rocks (for example talc or silicates), sugars (for example lactose), emulsifiers, dispersants (for example polyvinylpyrrolidone) and lubricants (for example magnesium sulfate).

In the case of oral administration, tablets may of course also contain additions such as sodium citrate together with additives such as starch,. gelatin and the like. Aqueous preparations for oral administration may furthermore be mixed with taste improvers or colors.

The preferred dosages on oral administration are from 0.001 to 5 mg/kg, preferably from 0.001 to 3 mg/kg, of body weight per 24 hours.

It may nevertheless be necessary where appropriate to deviate from the amounts mentioned, specifically as a function of the body weight, administration route, individual response toward the active compound, mode of preparation and time or interval over which administration takes place.

Unless indicated otherwise, all percent data in the examples below are based on weight; parts are parts by weight.

A. Evaluation of the Physiological Activity

The activity of the compounds according to the invention can be examined, for example, by the biological tests described below:

1. Inhibition of Human Recombinant Adenosine Kinase

The test is carried out on 96-well filter plates (DE81 from Millipore, DEAE cellulose, 0.65 μm). The test buffer contains 50 mM tris/HCl pH 8.0, 10 mM DTT and 1 mM MgCl₂; immediately prior to the start of the test, ATP is added in a concentration of 0.5 mM. The substrate used is ¹⁴C-adenosine, diluted 1:30 in cold adenosine; the final concentration of the cold adenosine is 4×10⁻⁶ M; the amount of ¹⁴C-adenosine is chosen such that, at a volume of 10 μl, an activity of 0.5 kBq (=30 000 decays per minute) is present per well of the filter plate. The substances to be tested are dissolved in DMSO in a concentration of 10⁻² M. Further dilutions are carried out using water.

When carrying out the test, initially 80 μl of buffer are initially charged into each well. The test substances, the substrate adenosine and finally human recombinant adenosine kinase (Mathews J. J. et al., Biochemistry (1998) 37, 15607-15620) in test buffer pH 7.4 are added successively, in each case in a volume of 10 μl. The adenosine kinase concentration is chosen such that at most 30% of the total amount of adenosine are converted. The plates are then incubated at 37° C. for 20 min. The reaction is then stopped by filtration with suction. The filters are washed in each case once with ice-cold wash buffer (15 mM Tris/HCl pH 8.5) and ethanol. The plate is dried at 37° C. (about 15 min). 50 μl of scintillation solution are then pipetted into each well, and the ¹⁴C activity is determined in a MicroBeta counter.

The inhibition of the enzyme activity by the substances tested is determined after substration of the blank value (no added enzyme) in comparison to the uninhibited enzyme (100% value).

The test results are stated as IC₅₀ values for the inhibition of adenosine kinase and listed in Table 1:

TABLE 1
Adenosine kinase inhibition (in vitro):

| Example | IC₅₀ [nM] |
|---|---|
| 4 | 50 |
| 6 | 20 |
| 8 | 80 |
| 10 | 10 |
| 13 | 100 |
| 24 | 30 |
| 29 | 80 |
| 32 | 30 |
| 35 | 20 |
| 37 | 20 |
| 193 | 30 |
| 198 | 20 |
| 201 | 20 |
| 255 | 5 |
| 270 | 30 |
| 326 | 15 |

2. In Vivo Test Model

Adult FBI (Foxhound Beagle Irish Setter) dogs (body weight 20-30 kg) are initially anesthetized using a combination of Trapanal 500 mg and Alloferin 55 mg. Anesthesia is maintained by infusion of a mixture of fentanyl 0.072 mg/kg, Alloferin 0.02 mg/kg and dihydrobenzpyridyl 0.25 mg/kg×min. Tubes are inserted into the animals, and they are ventilated with a mixture of O₂/N₂O (ratio 1:5) using an Engström ventilation pump with 16 breaths per min and a volume of 18-24 ml/kg. The body temperature is maintained at 38° C.±0.1° C. The arterial blood pressure is measured via a catheter in the femoral artery. A thoracotomy is carried out on the left side at the fifth intercostal space. The lung is pushed back and fixed and then incision in the pericardium is made. A proximal section of the LAD (left artery descendent) distally to the first diagonal branch is exposed, and a calibrated electromagnetic flow sensor (Gould Statham, Model SP7515) is placed around the vessel and linked to a flow meter (Statham, Model SP-2202). A mechanical occluder is placed distally to the flow sensor such that there are no branches between flow sensor and occluder.

Blood sampling and substance administration are carried out through a catheter in the femoral vein. A peripheral ECG is recorded using subcutaneously fixed needles. A microtip pressure manometer (Millar, Model PC-350) is pushed through the left atrium in order to measure left ventricular pressure. Measurement of the heart rate is triggered by the R wave of the ECG. The hemodynamic parameters and the coronary flow are recorded by a multi-channel recorder throughout the experiment.

An occlusion of four minutes causes a reactive hyperemia. The difference between the coronary flow under control conditions and the maximum flow during the reactive hyperemia is measured. The time required to reach half of this maximum flow during decrease is a suitable parameter to assess the reactive hyperemia.

Following a stabilization time of one hour, the experiment is started with a four-minute occlusion. 30 minutes later, the substance is administered (i.v.), and two minutes later, the vessel is reoccluded. Reactive hyperemia after verum and placebo is compared.

B. Working Examples

The present invention is illustrated by the following preferred examples, however, these examples do not limit the invention in any respect.

Abbreviations Used:

| | |
|---|---|
| DCI | Direct chemical ionization (with MS) |
| DMAP | 4-N,N-dimethylaminopyridine |
| DMF | N,N-dimethylformamide |
| DMSO | Dimethyl sulfoxide |
| EDC | N′-(3-dimethylaminopropyl)-N-ethylcarbodiimide × HCl |
| EI | Electron impact ionization (MS) |
| ESI | Electron spray ionization (MS) |
| HOBt | 1-hydroxy-1H-benzotriazol × H₂O |
| HPLC | High pressure, high performance liquid chromatography |
| MPLC | Medium pressure liquid chromatography |
| MS | Mass spectroscopy |
| NMR | Nuclear magnetic resonance spectroscopy |
| RP | Reverse phase (with HPLC) |
| RT | Room temperature |
| TFA | Trifluoroacetic acid |
| THF | Tetrahydrofuran |

Starting Materials:

### Example I

### Diethyl phenyl[trimethylsilyl)oxy]methylphosphonate

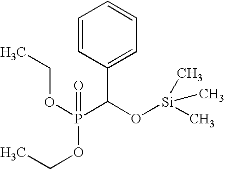

70.26 g (662.03 mmol) of benzaldehyde are added dropwise to a mixture of 100 g (601.84 mmol) of triethyl phosphite and 71.92 g (622.03 mmol) of chlorotrimethylsilane. The mixture is stirred at room temperature for 1 hour and then at 80° C. for 24 hours. The crude product is purified by fractional distillation. This gives 178.17 g (94%) of a colorless liquid of boiling point 110 to 113° C. (at 0.59 mbar).

¹H-NMR (200 MHz, DMSO-d₆): δ=0.0 (m, 9H), 1.1 (m, 6H), 3.7-4.02 (m, 4H), 5.05 (d, 1H), 7.15-7.45 (m, 5H).

### Example IIa

### 2-Hydroxy-2-(3,5-dimethoxyphenyl)-1-phenylethanone

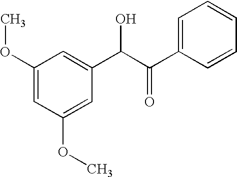

and

### Example IIb

### 1-(3,5-Dimethoxyphenyl-2-hydroxy-2-phenylethanone

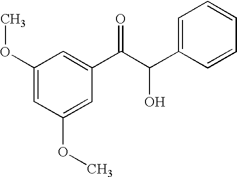

At −70° C. and under argon, 87.02 ml (139.24 mmol) of a 1.6 molar solution of n-butyllithium in hexane are added dropwise to a solution of 12.81 g (126.58 mmol) of diisopropylamine in 50 ml of THF. The mixture is stirred for another 30 minutes, and 40.05 g (126.58 mmol) of the compound from example I, dissolved in 50 ml of THF, are then added dropwise at −70° C. The mixture is stirred for another 30 min, and a solution of 18.93 g (113.92 mmol) of 3,5-dimethoxybenzaldehyde in 50 ml of THF is slowly added dropwise to the resulting suspension, and the mixture is stirred for 30 min. The mixture is warmed to room temperature, hydrolyzed dropwise using a 2 molar aqueous sodium hydroxide solution and stirred for another 1 h. Water is added and the mixture is extracted three times with diethyl ether. The combined organic phases are washed with 2 molar hydrochloric acid and sat. sodium chloride solution, dried and concentrated under reduced pressure. This gives about 43 g of an oil which, by crystallization from ethanol and flash or column chromatography on silica gel (mobile phase: cyclohexane/ethyl acetate) gives a total of 11.52 g (33%) of the compound IIa as colorless crystals of melting point 104 to 105° C. and 2.268 g (7%) of the compound IIb as colorless crystals of melting point 92 to 93° C.

### Example III

### 2-Amino-5-(3,5-dimethoxyphenyl)-4-phenylfuronitrile

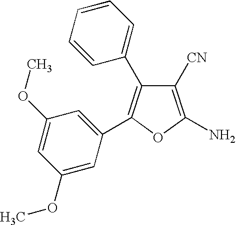

11.86 g (43.56 mmol) of the compound of example IIa are dissolved in 21.23 ml of DMF, 3.74 g (56.62 mmol) of malononitrile and 4.41 g (43.56 mmol) of triethylamine are added and the mixture is stirred at room temperature overnight. The mixture is then concentrated under reduced pressure and twice dissolved in toluene and reconcentrated, resulting in crystallization. The crystals are triturated with diethyl ether and a little ethyl acetate, filtered off with suction and washed with this mixture. The mother liquor is concentrated, again resulting in crystallization. The crystals are filtered off with suction, giving 10.2 g (73%) of light-beige crystals of melting point 179 to 181° C. which are used without further purification for the next step.

### Example IV

### Ethyl 3-cyano-5-(3,5,-dimethoxyphenyl)-4-phenyl-2-furanylimidoformate

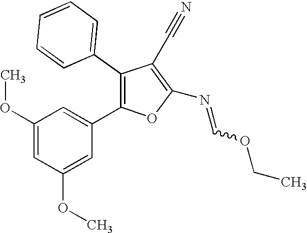

3.41 g (10.64 mmol) of the compound of example III in a mixture of 26.56 ml of ethyl orthoformate and 0.5 ml of acetic anhydride are heated under reflux for 5 hours, resulting in the formation of a solution. The reaction mixture is then concentrated under reduced pressure and purified by column chromatography (silica gel, mobile phase: cyclohexane/methylene chloride). This gives 3.62 g (90%) of beige crystals of melting point 103 to 104° C.

### Example V

### 6-(3,5-Dimethylphenyl)-5-phenylfuro[2,3-d]pyrimidin-4-(3H)-one

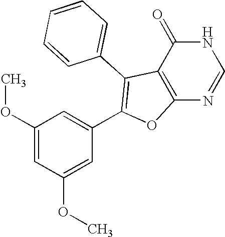

At 0° C., 30 ml of formic acid are added dropwise to 60 ml of acetic anhydride. The mixture is stirred at 0° C. for 30 min, 10.2g (31.84 mmol) of the compound of example III are added and the mixture is stirred under reflux. After 48 hours, the reaction mixture is cooled, giving the product in crystalline form, which can then be filtered off with suction and washed with diethyl ether. This gives 5.845 g (53%) of a colorless solid of melting point >250° C. From the filtrate, another 1.6 g (14%) of the product are isolated by repeating the reaction with 12 ml of acetic anhydride and 6 ml of formic acid.

¹H-NMR (400 MHz, DMSO-d₆): δ=3.61 (s, 6H), 6.46 (m, 1H), 6.55 (d. 2H), 7.38-7.48 (m, 5H), 8.17 (s, 1H), 12.64 (br. s, 1H).

### Example VI

### 4-Chloro-6-(3,5-dimethoxyphenyl)-5-phenylfuro[2,3-d]pyrimidine

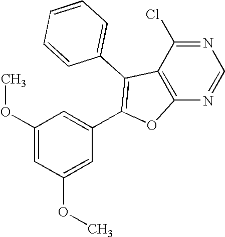

5.7 g (16.35 mmol of the compound of example V and 57 ml (611.53 mmol) of phosphoryl chloride are heated under reflux for 3 hours. The reaction mixture is then concentrated under reduced pressure, stirred with ice-water for 30 min and extracted with dichloromethane, and the extracts are dried and concentrated. This gives 5.6 g (93%) of a light-beige solid of melting point 138 to 140° C. which is used for the next step without further purification.

### Example VII

### 4-Chloro-6-(6-chloro-3-pyridinyl)-5-phenylfuro[2-3-d]pyrimidine

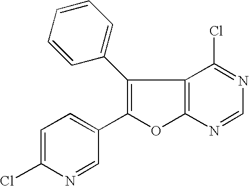

The ketone employed, 6-(6-chloro-3-pyridinyl)-5-phenylfuro[2,3-d]pyrimidin-4(3H)-one, is synthesized analogously to the preparation procedure of example V starting with 2-(6-chloro-3-pyridinyl)-2-hydroxy-1-phenylethanone which is synthesized analogously to the preparation procedure of examples IIa/IIb starting with 6-chloronicotinaldehyde.

Analogously to the reaction sequence for the synthesis of example VI, 2.6 g (8.03 mmol) of 6-(6-chloro-3-pyridinyl)-5-phenylfuro[2,3-d]pyrimidin-4(3H)-one are reacted in 26 ml of phosphoryl chloride. This gives 2.41 g (87.7%) of the product as colorless crystals which are reacted directly, without further purification.

### Example VIIIa

### 2-(4-Bromophenyl)2-hydroxy-1-phenylethanone

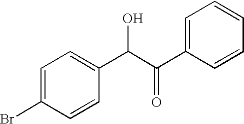

and

### Example VIIIb

### 1-(4-Bromophenyl)-2-hydroxy-2-phenylethanone

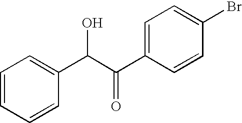

Analogously to the preparation of the compounds of examples IIa/IIb, 10.0 g (31.60 mmol) of the compound of example I are reacted with 5.26 g (28.44 mmol) of 4-bromobenzaldehyde. This gives 4 g (43%) of an isomer mixture, which is not separable, of example VIIIa and example VIIIb in the ratio 65:35 which is reacted directly in the next step.

¹H-NMR of the isomer mixture (200 MHz, CDCl₃): δ=4.51 (dd. 1H), 5.85-5.95 (m, 1H), 7.16-7.6 (m, 7H), 7.73=7.94 (m, 2H).

MS (DCIpos): m/z−308 (M+NH₄)⁺

### Example IXa

### 2-Amino-5-(4-bromophenyl)-4-phenyl-3-furonitrile

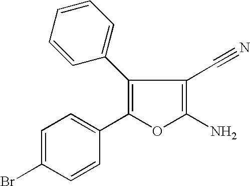

and

### Example IXb

### 2-Amino-4-(4-bromophenyl)-5-phenyl-3-furonitrile

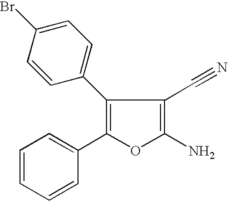

4.0 g (13.74 mmol) of the product mixture of examples VIIIa/VIIIb are reacted analogously to the preparation of the compound of example III. This gives 4.6 g (99%) of an isomer mixture, which is not separable, of example IXa and example IXb in the ratio 65: 35 which is directly reacted in the next step.

¹H-NMR of the isomer mixture (300 MHz, DMSO-d₆): δ=7.1-7.17 (m, 2H), 7.34-7.53 (m, 7H), 7.79 (s, 2H).

MS (EIpos): m/z−339.2 (M)⁺

### Example Xa

### 5-(4-Bromophenyl)-3-cyano-5-phenyl-2-furylimidoethylformate

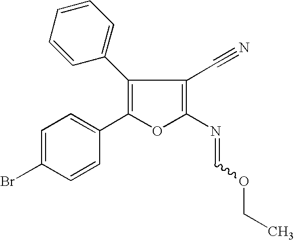

and

### Example Xb

### 4-(4-Bromophenyl)-3-cyano-4-phenyl-2-furylimidoethylformate

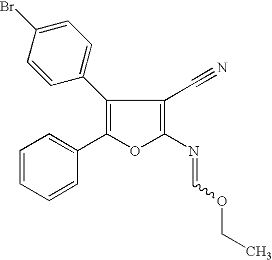

4.6 g (13.56 mmol) of the product mixture of examples IXa/IXb are reacted analogously to the preparation of the compound of example IV. This gives 3.9 g (69%) of an isomer mixture, which is not separable, for example Xa and example Xb in the ratio of 65:35 which is directly reacted in the next step.

¹H-NMR of the isomer mixture (300 MHz, CDCl₃): δ=1.43 (t, 3H), 4.47 (q, 2H), 7.23-7.45 (m, 8H), 7.52-7.58 (m, 1H), 8.45 (s, 1H).

MS (DCIpos): m/z=413 (M+NH₄)⁺

### Example Xa

### 6-(4-Bromophenyl)-N-isobutyl-5-phenylfuro[2,3-d]pyrimidine-4-amine

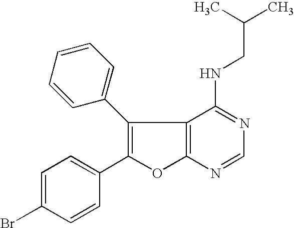

and

### Example XIb

### 5-(4-Bromophenyl)-N-isobutyl-6-phenylfuro[2,3-d]pyrimidine-4-amine

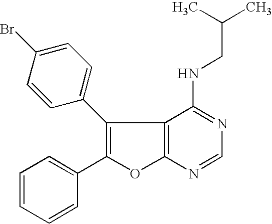

2.2 g (5.57 mmol) of the product mixture of the compounds of example Xa and example Xb are dissolved in 110 ml of ethanol, 2.44 g (33.4 mmol) of isobutylamine are added and the mixture is stirred at 40° C. for 1 hour. 33 ml of 1 molar aqueous sodium hydroxide solution are then added to the mixture, and the mixture is stirred at 90° C. for 1 hour. The resulting precipitate is filtered off with suction and the filtrate is separated by preparative RP-HPLC (Column: YMC Gel ODS-AQ S-11 μm, 250×30 mm; mobile phase: acetonitrile/water; flow rate: 50 ml/min, UV detection at 210 nm). This gives 1.0 g (39%) of the compound of example XIa and 450 mg (19%) of the compound of example XIb in the form of amorphous solids.

¹H-NMR of example XIa (300 MHz, CDCl₃): δ=0.75 (d, 6H), 1.68 (sept., 1H), 3.24 (t, 2H), 4.66 (br. t, 1H), 7.34-7.61 (m, 9H), 8.40 (s, 1H).

MS (ESIpos): m/z=424.3 (M+H)⁺

¹H-NMR of example XIb (300 MHz, CDCl₃): δ0.82 (d, 6H), 1.72 (sept., 1H), 3.28 (t. 2H), 4.61 (br. t. 1H), 7.25-7.721 (4m, 9H), 8.40 (s, 1H).

MS (EIpos): m/z=424.4 (M+H)⁺

### Example XII

### 6-(4-Bromophenyl)-4-chloro-5-phenylfuro[2,3-d]pyrimidine

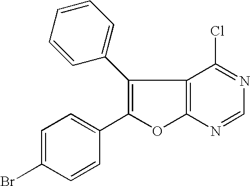

The ketone used, 6-(4-bromophenyl)-5-phenylfuro[2,3-d]pyrimidin-4(3H)-one, is synthesized analogously to the preparation of the compound of example V starting with the mixture of the compounds from example VIIIa/VIIIb (65:35).

A suspension of 1630 mg (4.44 mmol) of 6-(4-bromophenyl)-5-phenylfuro[2,3-d]-pyrimidin-4(3H)-one in 7 ml (75.10 mmol) of phosphoryl chloride is heated at reflux (oil bath temperature 135° C.) for 2 hours. After cooling, the solution is poured onto ice and made slightly alkaline using 25% strength aqueous ammonium solution. The resulting precipitate is filtered off with suction, washed with DMSO and dried under reduced pressure. This gives 680 mg (38%) of product as a light-beige solid.

¹H-NMR (300 MHz, DMSO-d₆): δ=7.38-7.68 (9H, m), 8.89 (1H, s).

MS (ESIpos): m/z=386 (M+H)⁺

### Example XIII

### 6-(4-Bromophenyl)4-chloro-5(4-fluorophenyl)furo[2,3-d]pyrimidine (Isomer mixture)

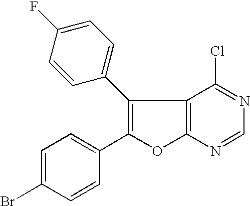

The ketone used, 6-(4-bromophenyl)-5-(4-fluorophenyl)-furo[2,3-d]pyrimidin-4(3H)-one, is synthesized analogously to the preparation of the compound of example V starting with the corresponding benzoin 2-(4-bromophenyl)-1-(4-fluorophenyl)-2-hydroxyethanone, which is employed as a mixture with 1-(4-bromophenyl)-2-(4-fluorophenyl)-2-hydroxyethanone in a ratio of 5:4. This mixture of the two isomeric benzoins is synthesized analogously to the procedure of the synthesis of the compounds of examples VIIIa/VIIIb starting with 4-bromobenzaldehyde and diethyl 4-fluorophenyl[trimethylsilyl)oxy]-methylphosphonate (prepared analogously to the compound of example I from 4-fluorobenzaldehyde).

Analogously to the preparation of the compound of example VI, 19.0 g (49.3 mmol) of 6-(4-bromophenyl)-5-(4-fluorophenyl)furo[2,3-d]pyrimidin-4(3H)-one are reacted with 67 ml (718.8 mmol) of phosphoryl chloride. This gives 19.7 g (94%) of the product as an isomer mixture [(6-(4-bromophenyl)-4-chloro-5-(4-fluorophenyl)furo[2,3-d]pyrimidine/5-(4-bromophenyl)-4-chloro-6-(4-fluorophenyl)-furo[2,3-d]pyrimidine=5:4] in the form of a white-brown solid.

¹H-NMR (300 MHz, DMSO-d₆) of the isomer mixture: δ=7.29-7.78 (8H, m), 8.88 (1H, s, Isomer), 8.89 (1H, s).

MS(EIpos): m/z=404 (M+H)⁺

### Example XIV

### 4-Chloro-5-(4-fluorophenyl)-6-phenylfuro[2,3-d]pyrimidine

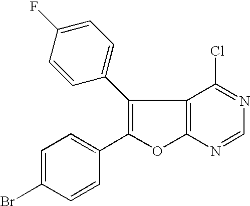

The ketone used, 5-(4-fluorophenyl)-6-phenylfuro[2,3-d]pyrimidin-4(3H)-one, is synthesized analogously to the preparation of the compound of example V starting with the corresponding benzoin 1-(4-fluorophenyl)-2-hydroxy-2-phenylethanone. The benzoin is synthesized analogously to the procedure of the synthesis of the compound of examples VIIIa/VIIIb starting with benzaldehyde and diethyl 4-fluorophenyl[trimethylsilyl)oxy]methylphosphonate (prepared analogously to the compound of example I from 4-fluorobenzaldehyde). Here, it is possible to obtain the desired isomer used for the synthesis by crystallization from ethanol (melting point 107 to 190° C.).

Analogously to the preparation of the compound of example VI , 7.9 g (25.79 mmol) of 5-(4-fluorophenyl)-6-phenylfuro[2,3-d]pyrimidin-4(3H)-one are reacted in 80 ml of phosphoryl chloride. This gives 7.65 g (91.3%) of the product as colorless crystals of melting point 124 to 127° C. which are directly reacted further.

### Example XV

### 4-Chloro-5,6-bis(4-methoxyphenyl)furo[2,3-d]pyrimidine

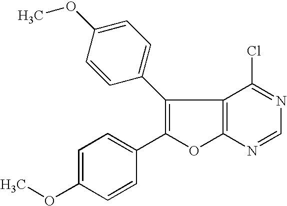

The synthesis of the ketone used, 5-6-bis(4-methoxyphenyl)furo[2,3-d]pyrimidin-4(3H)-one, is carried out analogously to the preparation of the compound of example V starting with 2-hydroxy-1,2-bis(4-methoxyphenyl)ethanone.

A suspension of 1.02 g (2.93 mmol) of 5,6-bis-(4-methoxyphenyl)-furo[2.3-d]pyrimidin-4(3H)-one in 4.0 ml (42.9 mmol) of phosphoryl chloride is heated at reflux for 45 min. Customary work-up gives 1.05 g (89%) of product in the form of a white solid.

¹H-NMR (300 MHz, DMSO-d₆): δ=3.78 (3H, s), 3.84 (3H, s), 6.99 (2H, d), 7.09 (2H, d), 7.43 (2H, d), 7.49 (2H, d), 8.80 (1H, s).

MS (EIpos): m/z=367 (M+H)⁺

### Example XVI

### N-(Diphenylmethylene)-N-{4-[5-(4-fluorophenyl)-4-(4-methyl-1-piperazinyl)-furo[2,3-d]pyrimidin-6-yl]phenyl}amine

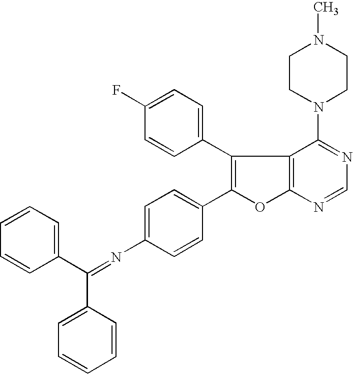

Under an argon atmosphere, 200 mg (0.428 mmol) of the compounds of example 21, 93.1 mg (0.51 mmol) of benzophenonemine and 57.6 mg (0.60 mmol) of sodium tertbutoxide are added to a suspension of 0.98 mg (0.001 mmol) of tris(dibenzylideneacetone)dipalladium(0) and 2.0 mg (0.003 mmol) of rac-2,2′-bis(diphenylphosphino)-1,1′-binaphthyl in 3 ml of dry toluene. The yellow reaction mixture is stirred at 80° C. for 3 hours. The mixture is then diluted with 10 ml of toluene and 10 ml of saturated aqueous sodium bicarbonate solution. The phases are separated and the organic phase is then dried over magnesium sulfate and concentrated under reduced pressure. The mixture is separated by preparative MPLC (Column: 50 g of silica gel; mobile phase: dichloromethane/ethanol 50:1 to 10:1; flow rate 80 ml/min; UV detection at 210 nm). Concentration under reduced pressure gives 190 mg (78%) of the product as a yellow solid.

¹H-NMR (300 MHz, DMSO-d₆): δ=1.97-2.09 (4H, m), 2.06 (3H, s), 3.08-3.21 (4H, m), 6.18 (2H, d), 7.10-7.22 (4H, m), 7.37-7.50 (10H, m), 7.61-7.69 (2H, m), 8.41 (1H, s).

MS (EIpos): m/z=568 (M+H)⁺

### Example XVII

### N-(5,6-Diphenylfuro[2,3-d]pyrimidin-4-yl)-N-isobutylamine

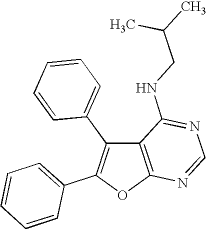

The product was prepared analogously to the preparation procedure of the compounds of examples XIa and XIb starting with 2-hydroxy- 1,2-diphenylethanone.

¹H-NMR (200 MHz, DMSO-d₆): δ=0.72 (d, 6H), 1.66 (sept., 1H), 3.20 (d, 2H), 4.89 (br. s. 1H), 7.25-7.68 (m, 10H), 8.32 (s, 1H).

MS (EIpos): m/z=344 (M+H)⁺

### Example XVIII

### 5-(4-Fluorophenyl)-4-(4-methyl-1-piperazinyl)-6-phenylfuro[2,3-d]pyrimidine

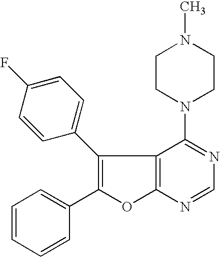

616.9 mg (6.16 mmol) of N-methylpiperazine are added to a suspension of 500 mg (1.54 mmol) of the compound of example XIV, and the mixture is stirred at boiling point for 4.5 h. 8 ml of water are then added, and the mixture is cooled to room temperature. The precipitate crystals are filtered off with suction and washed with ethanol/water (1:1). This gives, after drying, 384 mg (64.2%) of the product in the form of a light-yellow powder of melting point 143-144° C.

¹H-NMR (200 MHz, CDCl₃): δ=2.05-2.25 (m, 7H), 3.22-3.25 (m, 4H), 7.10-7.50 (m, 9H), 8.49 (s, 1H).

MS (EIpos): m/z=389 (M+H)⁺

### Example XIX

### 4-Chloro-6-(6-chloro-3-pyridinyl)-5-(4-fluorophenyl)furo[2,3-d]pyrimidine

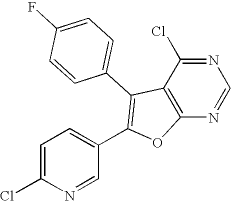

The synthesis of the ketone used, 6-(6-chloro-3-pyridiniyl-5-(4-fluorophenyl)-furo[2,3-d]pyrimidin-4(3H)-one, is carried out analogously to the preparation procedure of example V starting with 2-(6-chloro-3-pyridinyl)-2-hydroxy-1-(4-fluorophenyl)-ethanone, which is synthesized analogously to the preparation procedure of examples IIa/IIb starting with 6-chloronicotinaldehyde and 4-fluorobenzaldehyde.

Analogously to the reaction sequence for the synthesis of example VII, 3.34 g (9.77 mmol) of 6-(6-chloro-3-pyridinyl-5-(4-fluorophenyl)furo[2,3-d]pyrimidin-4(3H)-one are reacted in 35 ml of phosphoryl chloride. This gives 3.4 g (97%) of the product as a colorless to slightly yellow solid.

1H-NMR (300 MHz, DMSO-d₆): δ=7.36-7.46 (m, 2H), 7.56-7.67 (m, 3H), 7.89 (dd, 1H), 8.50 (d, 1H), 8.91 (s, 1H)

MS (ESIpos): m/z=360.2 (M+H)⁺

### Example XX

### 4-Chloro-6-(6-chloro-3-pyridinyl)-5-phenylfuro[2,3-d]pyrimidine

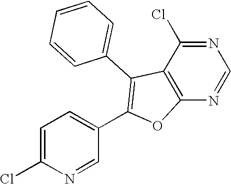

The synthesis of the ketone used, 6-(6-chloro-3-pyridinyl)-5-phenylfuro[2,3-d]pyrimidin-4(3H)-one, is carried out analogously to the preparation procedure of example V starting with 3-(6-chloro-3-pyridinyl-2-hydroxy-1-phenylethanone which is synthesized analogously to the preparation procedure of examples IIa/IIb starting with 6-chloronicotinaldehyde and benzaldhyde.

Analogously to the reaction sequence for the synthesis of example VII, 2.07 g (6.39 mmol) of 6-(6-chloro-3-pyridinyl)-5-phenylfuro[2,3-d]pyrimidin-4(3H)-one are reacted in 22.6 ml of phosphoryl chloride. This gives 1.48 g (68%) of the product as a colorless to slightly yellow solid.

MS (ESIpos): m/z=342 (M+H)⁺

### PREPARATION EXAMPLES

### Example 1

### N-Cycloheptyl-6-(3,5-dimethoxyphenyl)-5-phenylfuro[2,3-d]pyrimidine-4-amine

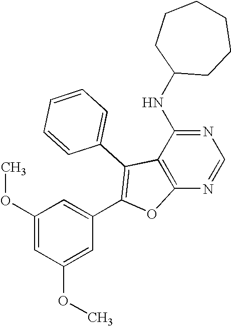

At 40° C., 100 mg (0.27 mmol) of the compound of example VI in 2 ml of ethanol are stirred with 180 mg (1.59 mmol) of cycloheptylamine for 2 hours, resulting in the formation of a precipitate. 2 ml of 1 N aqueous sodium hydroxide solution are then added and the mixture is stirred at 90° C. for 4 hours, whereupon a solution is formed again. The solution is stirred overnight and the crystalline product is then filtered off with suction and washed with water/ethanol. This gives 91 mg (77%) of colorless crystals of melting point 151 to 152° C.

¹H-NMR (300 MHz, CDCl₃): δ=1.22-1.6 (m, 10H), 1.75-188 (m, 2H), 3.61 (s, 6H), 4.2-4.34 (m, 1H), 4.65 (d, 1H), 6.35 (m, 1H), 6.7 (d, 2H), 7.47-7.61 (m, 5H), 8.39 (s, 1H).

MS (ESIpos): m/z=444 (M+H)⁺

### Example 2

### 6-(3,5-Dimethoxyphenyl)-5-phenyl-4-(1-piperidinyl)furo[2,3-d]pyrimidine

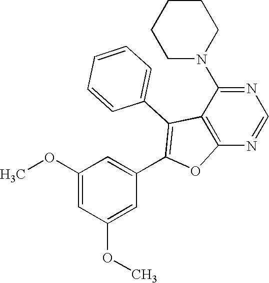

46.4 mg (0.55 mmol) of piperidine are added to a suspension of 50 mg (0.14 mmol) of the compound of example VI in 1 ml of ethanol, and the mixture is stirred at 80° C. for 3 hours. 1 ml of water is then added to the hot mixture, and the mixture is then cooled to room temperature. This results in the precipitation of the product in the form of crystals which are filtered off with suction and washed with ethanol/water 1:1. This gives 37 mg (65%) of colorless crystals of melting point 152 to 153° C.

¹H-NMR (300 MHz, CDCl₃): δ=1.12-1.29 (m, 4H), 1.34-1.5 (m, 2H), 3.2 (t. 4H), 3.6 (s, 6H), 6.35 (t, 1H), 6.62 (d, 2H), 7.35-7.55 (m, 5H), 8.45 (s, 1H).

MS (ESIpos): m/z−416 (M+H)⁺

### Example 3

### 5-(4-Fluorophenyl)-4-(4-methyl-1-piperazinyl)-6-(4-nitrophenyl)furo[2,3-d]pyrimidine

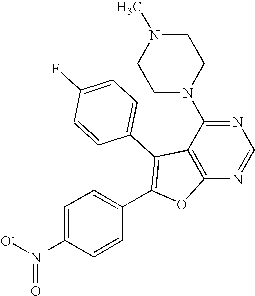

1 g (3.08 mmol) of the compound of example XIV are dissolved in warm acetonitrile and then cooled (ice/methanol cooling) with vigorous stirring. 613.4 mg (4.62 mmol) of nitronium tetrafluoroborate are then added to the mixture, the mixture is stirred at 0° C. for hour, 1.85 g (18.48 mmol) of 1-methylpiperazine are added and the mixture is heated at boiling point for 1.5 hours, resulting in the precipitation of a yellow solid. The reaction mixture is cooled and the solid is filtered off with suction and washed with acetonitrile. This gives 777.2 mg (58%) of a yellow product of melting point>250° C.

¹H-NMR (300 MHz, CDCl₃): δ=2.15 (t, 4H), 2.19 (s, 3H), 3.3 (t, 4H), 7.17-7.32 (m, 2H), 7.33-7.46 (m, 2H), 7.55-7.65 (m, 2H), 8.09-8.18 (m, 2H), 8.51 (s, 1H).

MS (ESIpos): m/z=434 (M+H)⁺

### Example 4

### 4-[5-(4-Fluorophenyl)-4-(4-methyl-1-piperazinyl)furo[2,3-d]pyrimidin-6-yl]aniline

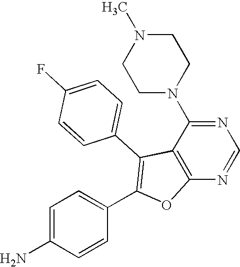

110 mg (0.25 mmol) of the compound of example 3 are dissolved in a mixture of 3 ml of methanol and 3 ml of THF, 11 mg of palladium on carbon (10% by weight) are added and the mixture is hydrogenated in atmosphere of hydrogen at atmospheric pressure. The mixture is then filtered through Celite, washed with THF and concentrated under reduced pressure. This gives 98 mg (96%) of colorless crystals of melting point 214 to 215° C. which were reacted without further purification.

MS (ESIpos): m/z=404 (M+H)⁺

### Example 5

### 6-(4-tert-Butyloxycarbonylaminomethylcarbonylaminophenyl)-5-(4-fluorophenyl)-4-(4-methyl-1-piperazinyl)furo[2,3-d]pyrimidine

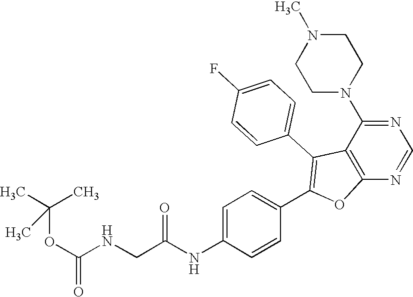

19.5 mg (0.11 mmol) of tert-butyloxycarbonylglycine and 28.5 mg (0.15 mmol) of EDC are added to a solution of 30 mg (0.07 mmol) of the compound from example 4 in 3 ml of THF and 2 ml of DMF, and the mixture is stirred at room temperature overnight. The mixture is then purified directly by column chromatography (silica gel, mobile phase: dichloromethane/methanol). Recrystallization from ethyl acetate gives 15.4 mg (37%) of the product as a colorless solid.

¹H-NMR (200 MHz, CDCl₃): δ=1.46 (s, 9H), 2.16 (t, 4H), 2.21 (s, 3H), 3.26 (t. 4H), 3.9 (d, 2H), 4.19 (br. s. 1H), 7.1-7.24 (m, 2H), 7.32-7.5 (m, 6H), 8.25 (br. s. 1H), 8.47 (s, 1H).

MS (ESIpos): m/z=561 (M+H)⁺

### Example 6

### N-1-{4-[5-(4-Fluorophenyl)-4-(4-methyl-1-piperazinyl)furo[2,3-d]pyrimidin-6-yl]phenylgylcinamide dihydrochloride

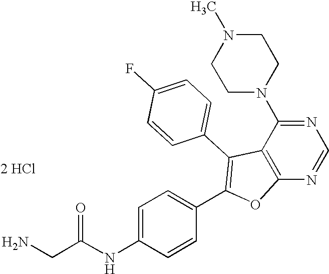

133 mg (0.24 mmol) of the compound from example 5 are stirred in 20 ml of 4 N HCl in dioxane for 2 hours, resulting in the precipitation of the product as a solid. The mixture is concentrated under reduced pressure and the solid is dissolved in a little water, filtered and dried. This gives 105 mg (83%) of a colorless powder.

¹H-NMR (400 MHz, D₂O): δ=2.56 (t, 2H), 2.84 (s, 3H), 3.08 (t, 2H), 3.31 (d, 2H), 3.71 (d, 2H), 4.02 (s, 2H), 7.29-7.46 (m, 8H), 8.34 (s, 1H).

MS (ESIpos): m/z=461 (M−2 HCl+H)⁺

### Example 7

### 4-(4-Methyl-1-piperazinyl)-6-[N-(4-morpholinyl)-3-pyridinyl]-5-phenylfuro[2,3-d]pyrimidine

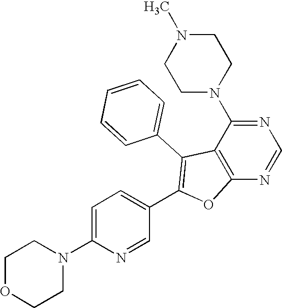

A mixture of 50 mg (0.12 mmol) of the compound from example 94 and 2 ml of morpholine is stirred at 130° C. for 1.5 hours. After cooling, the product precipitates as a solid. The mixture is diluted with ethyl acetate and the solid is filtered off with suction and then washed with ethanol and water. This gives 54 mg (96%) of light-beige crystals of melting point 255 to 257° C.

¹H-NMR (400 MHz, CDCl₃): δ=2.13 (s, 4H), 2.19 (s, 3H), 3.26 (t, 4H, 3.53 (t, 4H), 3.79 (t, 4H), 6.52 (d, 1H), 7.36-7.5 (m, 5H), 7.57 (dd, 1H), 8.28 (m, 1H), 8.45 (s, 1H).

MS (DCI): M/z=457 (M+H)⁺

### Example 8

### 5-[4-(4-Methyl-1-piperazinyl)-5-phenylfuro[2,3-d]pyrimidin-6-yl]-2-pyridineamine

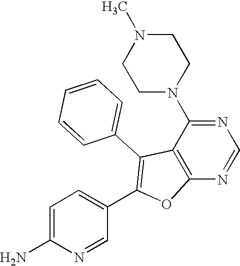

A mixture of 50 mg (0.12mmol) of the compound from example 94, 1.6 g of acetamide (27.09 mmol) and 200 mg of potassium carbonate is stirred at 210° C. for 32 hours. After cooling to 70° C., water and ethyl acetate are added, the aqueous phase is extracted with ethyl acetate and the combined organic phases are washed with water, dried and concentrated. The crude product is then triturated with a mixture of ethyl acetate and diethyl ether and filtered off. This gives 26.8 mg (56%) of beige crystals of melting point 229 to 231° C.

¹H-NMR (300 MHz, DMSO-d₆): δ=1.97 (t, 4H), 2.04 (s, 3H), 3.14 (t, 4H), 6.33 (s, 2H), 6.38 (d, 1H), 7.32-7.57 (m, 6H), 7.91 (d, 1H), 8.4 (s, 1H).

MS (ESIpos): m/z=387 (M+H)⁺

### Example 9

### 1-[6-(6-Chloro-3-pyridinyl)-5-phenylfuro[2,3-d]pyrimidin-4-yl]-4-piperidinol

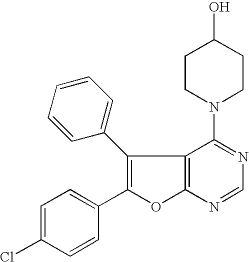

The compound is prepared in quantitative yield analogously to the procedure for the synthesis of example 2 from the compound of example VII and 4-piperidinol.

¹H-NMR (300 MHz, CDCl₃): δ=1.14-1.32 (m, 3H), 1.50-1.65 (m, 2H), 2.89-3.02 (m, 2H), 3.59-3.79 (m, 3H), 7.21-7.28 (m, 1H), 7.36-7.54 (m, 5H), 7.70-7.74 (m, 1H) 8.38-8.41 (m, 1H), 8.48 (s, 1H).

MS (ESIpos): m/z=407 (M+H)⁺

### Example 10

### 1-{6-[6-(4-Methyl-1-piperazinyl)-3-pyridinyl]-5-phenylfuro[2,3-d]pyrimidin-4-yl]-4-piperidinol

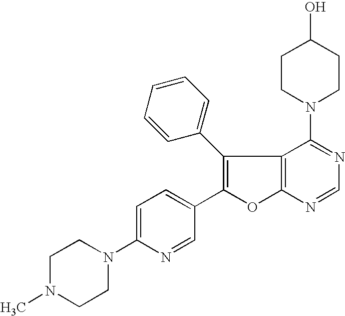

The compound was prepared analogously to the procedure for example 7 from example 9 and N-methylpiperazine (yield: 81%).

¹H-NMR (300 MHz, CDCl₃): δ=1.10-1.30 (m, 3H), 1.50-1.68 (m, 2H), 2.38 (s, 3H), 2.50-2.78 (m, 4H), 2.80-2.98 (m, 2H), 3.50-3.68 (m, 6H), 3.92-4.08 (m, 1H), 6.65 (d, 1H), 7.15-7.60 (m, 6H), 8.23-8.29 (m, 1H), 8.46 (s, 1H).

MS (ESIpos): m/z=471 (M+H)⁺

### Example 11

### Methyl 4-[4-(isobutylamino)-5-phenylfuro[2,3-d]pyrimidin-6-yl]benzoate

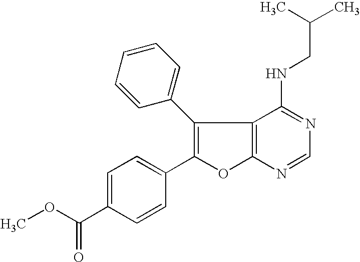

Under protective gas and with exclusion of oxygen, a mixture of 21.4 mg (0.05 mmol) of bis(diphenylphosphino)propane and 10.6 mg of palladium(II) acetate are initially charged in 2.0 ml of DMF. 200.0 mg (0.47 mmol) of the compound from example XIa (dissolved in a mixture of 4.0 ml of methanol and 4.0 ml of DMF) and 479.2 mg (4.74 mmol) of triethylamine are added, and the mixture stirred at 120° C. for 1 hour. The reaction mixture is then poured into water and extracted with methylene chloride. The combined organic phases are washed once with water and once with saturated sodium chloride solution, dried with sodium sulfate and concentrated under reduced pressure. The residue is purified by chromatography (silica gel, mobile phase: cyclohexane/ethyl acetate 1:1). This gives 137 mg (64.8%) of the product as an amorphous solid.

¹H-NMR (300 MHz, CDCl₃): δ=0.77 (d, 6H), 1.67 (sept., 1H), 3.26 (t, 2H), 3.89 (s, 3), 4.66-4.70 (m, 1H), 7.48-7.60 (m, 7H), 7.93 (d, 2H), 8.41 (s, 1H).

MS (ESIpos): m/z=402 (M+H)⁺

### Example 12

### 4-[4-(Isobutylamino)-5-phenylfuro[2.3-d]pyrimidin-6-yl]benzoic Acid

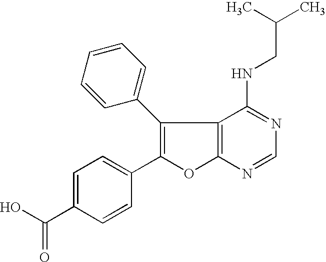

163.6 mg (6.83 mmol) of lithium hydroxide are added to a mixture of 590 mg (1.37 mmol) of the compound from example 11 in 14 ml of methanol and 5 ml of water, and the mixture is stirred at 50° C. for 4 hours. The methanol is then removed under reduced pressure and the aqueous phase that remains is, with ice-bath cooling, acidified with 1N aqueous hydrochloric acid. The mixture is concentrated under reduced pressure, resulting in the precipitation of the product. The crystals formed are washed with water and dried under reduced pressure. This gives 485 mg (91.6%) of the product as an amorphous solid which was directly reacted further.

### Example 13

### N-Isobutyl-6-{4-[(4-methyl-1-piperazinyl)carbonyl]phenyl}-5-phenylfuro[2,3-d]-pyrimidine-4-amine

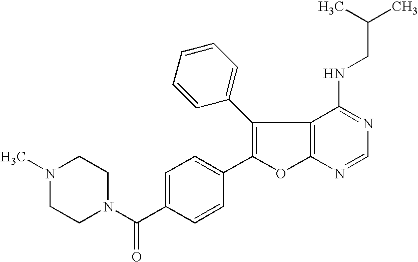

30.6 mg (0.23 mmol) of HOBTt, 45.5 mg (0.24 mmol) of EDC, 62.0 mg (0.62 mmol) of 1-methylpiperazine, 62.6 mg (0.62 mmol) of N-methylmorpholine and a catalytic amount of DMAP are added successively to a solution of 80.0 mg (0.21 mmol) of the compound from example 12 in 5 ml of DMF, and the reaction mixture is stirred at room temperature overnight. The mixture is separated by preparative R-HPLC (column: YMC Gel ODS-AQ S-11 μm, 250×30 mm; mobile phase: acetonitrile/water; flow rate: 50 ml/min; UV detection at 210 nm). This gives, after concentration under reduced pressure, 52 mg (53.6%) of the product as a colorless solid.

¹H-NMR (300 MHz, CDCL₃): δ=0.76 (s, 3H), 0.78 (s, 3H), 1.5-1.75 (m, 1H), 2.3 (s, 3H), 2.3-2.55 (br.s, 4H), 3.26 (m, 2H), 3.34-3.88 (m, 4H), 4.65-4.70 (t, 1H), 7.25-7.33 (d, 2H), 7.47-7.59 (m, 7H), 8.41 (s, 1H).

MS (ESIpos): m/z=470 (M+H)⁺

### Example 14

### 2-(4-{4-[4-(Isobutylamino)-5-phenylfuro[2,3-d]pyrimidin-6-yl]phenyl}-1-piperazinyl)ethanol

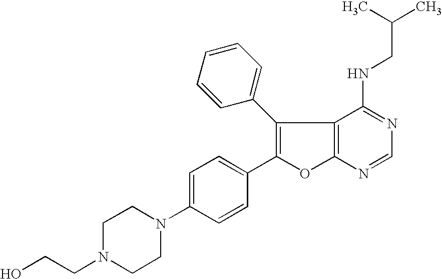

Under protective gas and with rigorous exclusion of oxygen, 75.0 mg of the compound of the example XIa, 4.88 mg (0.005 mmol) of tris(dibenzylideneacetone)dipalladium, 11.0 mg (0.018 mmol) of rac-2,2′-bis(diphenylphosphino)-1,1′-binaphthyl and 23.9 mg (0.21 mmol) of potassium tert-butoxide are initially charged in 5 ml of toluene, and 69.3 mg (0.53 mmol) of 2-(1-piperazinyl)ethanol are added. The reaction mixture is stirred at 60° C. for 2 hours, another charge of the catalytic amount of tris(dibenzylideneacetone)dipalladium stated above and rac-BINAP is added, and the mixture is stirred at 60° C. overnight. The solvent is removed under reduced pressure and the mixture is separated by preparative HPLC (Column: YMC Gel ODS-AQ S-11 μm, 250×30 mm; mobile phase: acetonitrile/water; flow rate: 50 ml/min; UV detection at 210 nm). This gives, after concentration under reduced pressure, 76 mg (90%) of the product as a colorless solid.

¹H-NMR (300 MHz, DMSO-d₆): δ=0.74 (d, 6H), 1.18 (sept., 1H), 2.40 (t, 2H), 3.13-3.34 (m, 10H), 3.47-3.59 (m, 2H), 4.38 (t, 1H), 4.78 (t, 1H), 6.85 (d, 2H), 7.27 (d, 2H), 7.51-7.61 (m, 5H), 8.27 (s, 1H).

MS (ESIpos): m/z=472 (M+H)⁺

### Example 15

### N-Isobutyl-6-(4-nitrophenyl)-5-phenylfuro[2,3-d]pyrimidine-4-amine

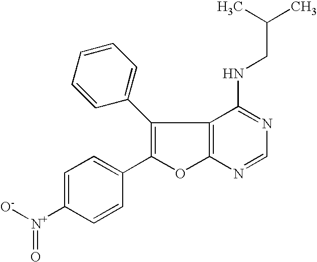

At 0° C., 352.7 mg (1.75 mmol) of nitronium tetrafluoroborate are added a little at a time to a solution of 500 mg (1.46 mmol) of the compound of example XVII in 25.0 ml of dichloromethane, and the reaction mixture is stirred at 0° C. for 1 hour and then at room temperature overnight. Water is added and the reaction mixture is then extracted twice with dichloromethane and the organic phases are combined, washed once with saturated sodium chloride solution, dried over sodium sulfate and concentrated under reduced pressure. Chromatography of the residue (silica gel, mobile phase: cyclohexane/ethyl acetate) gives 414 mg (66.6%) of the product as a solid.

¹H-NMR (200 MHz, DMSO-d₆): δ=0.73 (d, 6H), 1.68 (sept., 1H), 3.23 (t, 2H), 5.03 (br. t. 1H), 7.55-7.72 (m, 7H), 8.15-8.27 (m, 2H), 8.41 (s, 1H).

MS (DCIpos): m/z=389 (M+H)⁺

### Example 16

### 6-(4-Aminophenyl)-N-isobutyl-5-phenylfuro[2,3-d]pyrimidine-4-amine

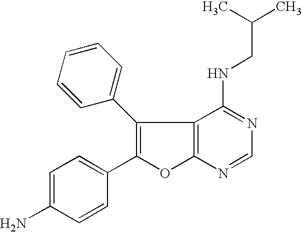

0.25 g (0.13 mmol) of palladium on carbon (10% by weight) is added to a solution of 5.0 g (12.87 mmol) of the compound from example 15 in 102 ml of THF and 102 ml of methanol, and the mixture is hydrogenated at room temperature in an atmosphere of hydrogen at 1 bar overnight. The mixture is then filtered through Celite and concentrated under reduced pressure. Chromatography of the residue (silica gel, mobile phase: cyclohexane/ethyl acetate) gives 1.84 g (39%) of the product in the form of light-beige crystals.

¹H-NMR (200 MHz, CDCl₃): δ=0.77 (d, 6H), 1.67 (sept., 1H), 3.24 (t, 2H), 3.80 (br. s, 2H), 4.58-4.63 (m, 1H), 6.55 (d, 2H), 7.33 (d, 2H), 7.47-7.54 (m, 5H), 8.36 (s, 1H).

MS (DCIpos): m/z=359 (M+H)⁺

### Example 17

### N-{4-[4-(Isobutylamino)-5-phenylfuro[2,3-d]pyrimidin-6-yl]phenyl}-N′,N″-dimethylglycinamide

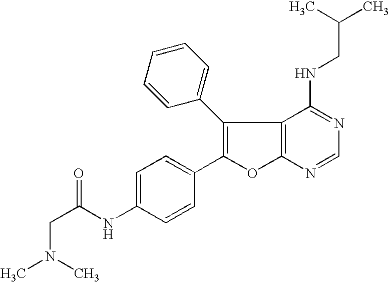

41.4 mg (0.31 mmol) of HOBt and 61.5 mg (0.32 mmol) of EDC are added to a solution of 43.1 mg (0.42 mmol) of N,N-dimethylglycine in 5.0 ml of DMF, and the mixture is stirred at room temperature for 30 min. 100.0 mg (0.28 mmol) of the compound from example 16, 112.8 mg (1.12 mmol) of N-methylmorpholine and 3.41 mg (0.28 mmol) of DMAP are then added, and the mixture is stirred overnight. The crude mixture is then separated directly by preparative HPLC (Column: YMC Gel ODS-AQ S-11 μm, 250×30 mm; mobile phase: acetonitrile/water; flow rate: 50 ml/min; UV detection at 210 mn). Further separation by column chromatography (silica gel, mobile phase: methylene chloride/methanol) gives, after concentration under reduced pressure, 49 mg (37%) of the product as a colorless solid.

¹H-NMR (200 MHz, DMSO-d₆): δ=0.72 (d, 6H), 1.61 (sept., 1H), 2.25 (s, 6H), 3.06 (s, 2H), 3.21 (t, 2H), 4.89 (t, 1H), 7.35 (d, 2H), 7.53-7.67 (m, 7H), 8.32 (s, 1H), 9.87 (s, 1H).

MS (DCIpos): m/z=444 (M+H)⁺

### Example 18

### N-{4-[4-(Isobutylamino)-5-phenylfuro[2,3-d]pyrimidin-6-yl]phenyl}methanesulfonamide

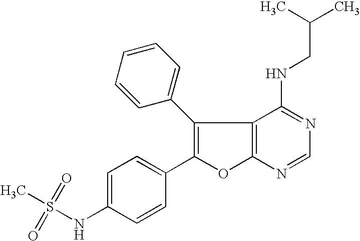

23.0 mg (0.20 mmol) of methanesulfonyl chloride are added to a solution of 60.0 mg (0.17 mmol) of the compound from example 16 in 2.0 ml of pyridine. The reaction mixture is stirred at room temperature overnight and then separated directly by preparative HPLC (Column: YMC Gel ODS-AQ S-11 μm, 250×30 mm; mobile phase: acetonitrile/water; flow rate: 50 ml/min; UV detection at 210 nm). This gives 67 mg (90%) of the product as a colorless solid.

¹H-NMR (300 MHz, CDCl₃): δ=0.78 (d, 6H), 1.67 (sept., 1H), 3.04 (s, 3H), 3.25 (t, 2H), 4.65 (t, 1H), 7.12 (d, 2H), 7.45-7.6 (m, 7H), 8.40 (s, 1H).

MS (ESIpos): m/z=437 (M)⁺

### Example 19

### tert-Butyl 4-[4-(isobutylamino-5-phenylfuro[2,3-d]pyrimidin-6-yl]phenyl-carbamate

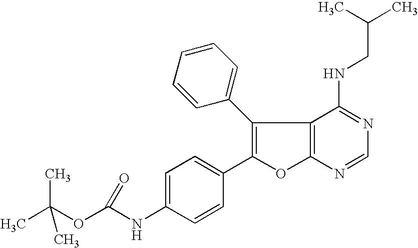

66.9 mg (0.31 mmol) of di-tert-butyl pyrocarbonate and 73.9 mg (0.7 mmol) of sodium carbonate are added to a solution of 100.0 mg (0.28 mmol) of the compound from example 16 in 2.0 ml of water and 1.0 ml of THF, and the mixture is stirred overnight. The organic solvent is then removed under reduced pressure and the mixture is extracted with dichloromethane. Since the reaction was incomplete, the mixture is reconcentrated under reduced pressure and taken up in 3 ml of dioxane, 66.98 mg (0.31 mmol) of di-tert-butyl pyrocarbonate are added and the mixture is again stirred overnight. Following concentration under reduced pressure, the mixture is separated directly by preparative HPLC (Column: YMC Gel ODS-AQ S-11 μm, 250×30 mm; mobile phase: acetonitrile/water; flow rate: 60 ml/min; UV detection at 210 nm). This gives 49 mg (38%) of the product as a colorless solid.

¹H-NMR (300 MHz, CDCl₃): δ=0.73 (d, 6H), 1.47 (s, 9H), 1.65 (sept., 1H), 3.20 (t, 2H), 4.85 (t, 1H), 7.31 (d, 2H), 7.40 (d, 2H), 7.5-7.64 (m, 5H), 8.30 (s, 1H).

MS (ESIpos): m/z=459 (M)⁺

### Example 20

### N-{4-[4-(Isobutylamino)-5-phenylfuro[2,3-d]pyrimidin-6-yl]phenyl}-N′-(4-methoxyphenyl)urea

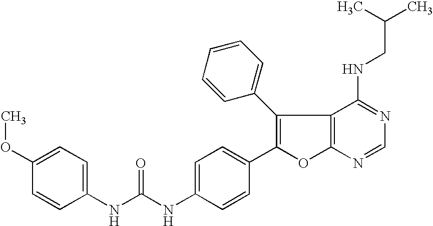

At 0° C., 50.8 mg (0.5 mmol) of triethylamine and a catalytic amount of DMAP are added to a solution of 60.0 mg (0.17 mmol) of the compound from example 16 in 3.0 ml of dichloromethane. 37.4 mg (0.25 mmol) of 1-isocyanato-4-methoxybenzene are then added dropwise, and the mixture is stirred at room temperature for 24 h. The reaction mixture is poured into water and extracted with dichloromethane. The organic phase is then washed with saturated sodium chloride solution, dried over sodium sulfate and concentrated under reduced pressure. The product is then separated directly by preparative HPLC (Column: YMC Gel ODS-AQ S-11 μm, 250×30 mm; mobile phase: acetonitrile/water; flow rate: 50 ml/min; UV detection at 210 nm). This gives 37 mg (44%) of the product as a colorless solid.

¹H-NMR (300 MHz, CDCl₃): δ=0.74 (d, 6H), 1.66 (sept., 1H), 3.21 (t, 2H), 3.71 (s, 3H), 4.85 (t, 1H), 6.85 (d, 2H), 7.28-7.38 (m, 4H), 7.39-7.45 (m, 2H), 7.53-7.65 (m, 5H), 8.3 (s, 1H), 8.5 (s, 1H), 8.72 (s, 1H).

MS (ESIpos): m/z=508 (M)⁺

### Example 21

### 6-(4-Bromophenyl)-5-(4-fluorophenyl)-4-(4-methyl-1-piperazinyl)furo[2,3-d]pyrimidine

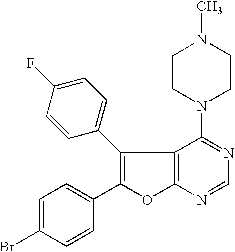

3.00 g (7.4 mmol) of the compound of example XIII (isomer mixture 5:4) are suspended in 60 ml of dioxane and, after addition of 4.47 g (44.6 mmol) of 1-methylpiperazine, heated at reflux (oil bath temperature 82° C.) for 12 hours. After customary work-up, the resulting residue is separated by preparative HPLC (Column: 250×20 mm Kromasil 100 C-18, 5 μm; mobile phase: water/acetonitrile/1% strength trifluoroacetic acid, 44:45:11; flow rate: 25 ml/min; UV detection at 210 nm). This gives 957 mg (28%) of the product in the form of a slightly yellow solid, in addition to 344 mg (10%) of the corresponding isomers.

¹H-NMR (300 MHz, DMSO-d₆): δ=2.13 (4H, t), 2.20 (3H, s), 3.29 (4H, t), 7.12-7.47 (8H, m), 8.48 (1H, s).

MS (ESIpos): m/z=468 (M)⁺

### Example 22

### 6-(4-Bromophenyl)-4-(4-methyl-1-piperazinyl)-5-phenylfuro[2.3-d]pyrimidine

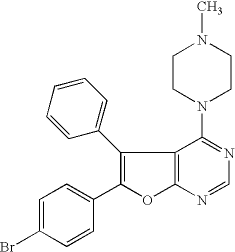

1.35 g (9.80 mmol) of potassium carbonate are added to a suspension of 630 mg (1.63 mmol) of the compound of example XII in 50 ml of tetrahydrofuran. After addition of 654 mg (6.53 mmol) if 1-methylpiperazine, the suspension is heated at reflux (oil bath temperature 88° C.) for 90 min. After cooling, 20 ml of water and 20 ml of ethyl acetate are added to the reaction solution. The aqueous phase is extracted three times with ethyl acetate. The combined organic phases are washed twice with saturated-aqueous sodium chloride solution, dried over magnesium sulfate and concentrated under reduced pressure. This gives 710 mg (88%) of the product in the form of a white solid.

¹H-NMR (300 MHz, DMSO-d₆): δ=1.91-2.08 (4H, m), 2.03 (3H, s), 3.09-3.21 (4H, m), 7.24-7.36 (2H, m), 7.38-7.61 (7H, m), 8.46 (1H, s).

MS (ESIpos): m/z=460 (M+H)⁺

### Example 23

### 1-[6-(4-Bromophenyl)-5-(4-fluorophenyl)furo[2,3-d]pyrimidin-4-yl]-4-piperidinol

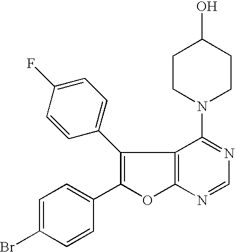

Analogously to the reaction of example 21, 2.00 g (4.95 mmol) of the compound of example XIII (isomer mixture) are reacted with 2.00 g (19.82 mmol) of 4-hydroxypiperidine. This gives 1.50 g (64%) of the product as a white solid.

¹H-NMR (300 MHz, DMSO-d₆): δ=0.98-1.12 (2H, m), 1.38-1.50 (2H, m), 2.82-2.97 (2H, m), 3.45-3.59 (3H, m), 4.48 (1H, d), 7.29 (2H, d), 7.32-7.53 (4H, m), 7.57 (2H, d), 8.43 (1H, s).

MS (ESIpos): m/z=469 (M+H)⁺

### Example 24

### 1-{4-[5-(4-Fluorophenyl)-4-(4-methyl-1-piperazinyl)furo[2,3-d]pyrimidin-6-yl]phenyl}-2-azetidinone

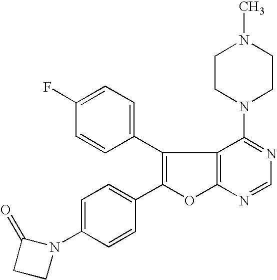

In a Schlenk tube which had been baked out, 2.0 mg (0.002 mmol) of tris(dibenzylideneacetone)dipalladium, 3.7 mg (0.006 mmol) of 9,9-dimethyl-4,5-bis(diphenylphosphino)xanthene, 18.3 mg (0.251 mmol) of 2-azetidinone, 97.6 mg (97.61 mmol) of cesium carbonate and 100.0 mg (0.21 mmol) of the compound from example 21 are initially charged under argon. After further flushing with argon, 0.30 ml of dry dioxane are added. The suspension, which is initially reddish and later yellow, is heated at 100° C. for 48 h, with vigorous stirring. After cooling, dichloromethane (about 5 ml) is added to the reaction mixture and the mixture is filtered and concentrated under reduced pressure. The mixture is separated by preparative RP-HPLC (Column: YMC Gel ODS-AQ S-11 μm, 250×30 mm; mobile phase: acetonitrile/water; flow rate: 50 ml/min; UV detection at 210 nm). This gives, after concentration under reduced pressure, 39.0 mg (39.8%) of the product as a colorless solid.

¹H-NMR (300 MHz, CDCl₃): δ=2.11-2.17 (4H, m), 2.19 (3H, s), 3.19 (2H, t), 3.22-3.31 (4H, m), 3.64 (2H, t), 7.14-7.21 (2H, m), 7.23-7.30 (2H, m), 7.33-7.45 (m, 4H), 8.48 (1H, s).

MS (EIpos): m/z=458.4 (M+H)⁺

### Example 25

### 4-[5-(4-Fluorophenyl)-4-(4-methyl-1-piperazinyl)furo[2,3-d]pyrimidin-6-yl]-phenylamine

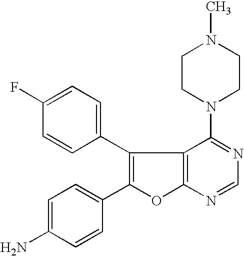

At 60° C., 249.9 mg (3.96 mmol) of ammonium formate and 14.1 mg (0.013 mmol) of palladium on carbon (10% by weight) are added to a solution of 150 mg (0.26 mmol) of the compound of example XVI in 3 ml of methanol. The reaction mixture is stirred at the stated temperature for 4 hours and then filtered through a Seitz filter. The filtrate is concentrated under reduced pressure, dissolved in DMSO and then separated by preparative RP-HPLC (Column: YMC Gel ODS-AQ S-11 μm, 250×30 mm; mobile phase: acetonitrile/water; flow rate: 50 ml/min; UV detection at 210 nm). This gives, after concentration under reduced pressure, 89 mg (65%) of the product as TFA salt in the form of a colorless solid.

¹H-NMR (300 MHz, DMSO-d₆): δ=2.73 (3H, s), 2.81-3.01 (2H, m), 3.12-3.29 (2H, m), 3.60-3.85 (4H, m), 6.50 (2H, d), 7.09 (2H, d), 7.29-7.54 (4H, m), 8.48 (1H, s), 9.55 (2H, br s).

MS (EIpos): m/z=404 (M+H)⁺

### Example 26

### N-(4-{[5,6-Bis(4-methoxyphenyl)furo[2,3-d]pyrimidin-4-yl]amino}transcyclohexyl)-5-oxotetrahydro-2-furancarboxylamide

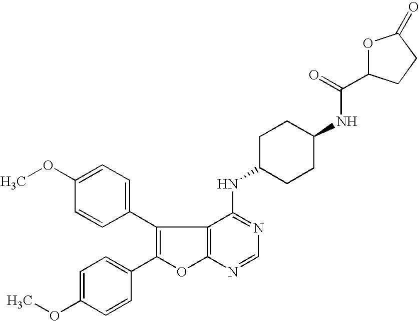

120 mg (0;27 mmol) of the compound from example 168, 56.9 mg (0.297 mmol) of EDC, 40.1 mg (0.297 mmol) of HOBt and 109 mg (1.08 mmol) of triethylamine are added to a solution of 35;1 mg (0;270 mmol) of 5-oxotetrahydro-2-furancarboxylic acid in 2 ml of dichloromethane. The mixture is stirred at room temperature for 24 hours and, after addition of 2 ml of saturated aqueous sodium bicarbonate solution, filtered through Extrelut, and the filtrate is concentrated under reduced pressure. The mixture is separated by MPLC (Column: 50 g of silica gel; mobile phase: dichloromethane/ethanol 20:1; flow rate 80 ml/min; UV detection at 210 nm). This gives 50 mg (33%) of the product as a colorless solid.

¹H-NMR (300 MHz, DMSO-d₆): δ=0.94-1.12 (2H, m), 1.21-1.42 (2H, m), 1.65-1.78 (2H, m), 1.88-2.11 (3H, m), 2.25-2.59 (3H, m), 3.46-3.61 (1H, m), 3.75 (3H, s), 3.80-3.94 (m, 1H), 3.86 (3H, s), 4.19 (1H, d), 4.80 (1H, dd), 6.94 (2H, d), 7.17 (2H, d), 7.40 (2H, d), 7.44 (2H, d), 8.08 (1H, d), 8.31 (1H, s).

MS (EIpos): m/z=557 (M+H)⁺

### Example 27

### N-(4-{[5,6-Bis(4-methoxyphenyl)furo[2,3-d]pyrimidin-4-yl]amino}transcyclohexyl)methanesulfonamide

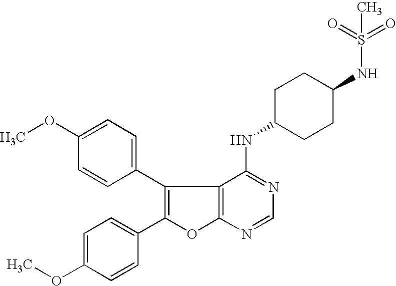

A solution of 47.6 mg (0.11 mmol) of the compound from example 168 in 0.5 ml of dichloromethane and 43.3 mg (0.43 mmol) of triethylamine is added dropwise to a solution of 12.3 mg (0.11 mmol) of methanesulfonyl chloride in 2 ml of dichloromethane. A spatula tip of 4-dimethylaminopyridine is added, and the mixture is then stirred at room temperature for 24 h. 2 ml of saturated aqueous sodium bicarbonate solution are added and the mixture is filtered through Extrelut. The filtrate is concentrated under reduced pressure and the mixture is separated directly by preparative HPLC (Column: YMC Gel ODS-AQ S-11 μm, 250×30 mm; mobile phase: acetonitrile/water; flow rate: 50 ml/min; UV detection at 210 nm). This gives 20.1 mg (35.2%) of the product as a colorless solid.

¹H-NMR (300 MHz, DMSO-d₆): δ=0.94-1.10 (2H, m), 1.21-1.40 (2H, m), 1.76-1.97 (4H, m), 2.88 (3H, s), 3.02-3.17 (1H, m), 3.75 (3H, s), 3.78-3.89 (1H, m), 3.86 (3H, s), 4.15 (1H, d), 6.90-6.99 (3H, m), 7.16 (2H, d), 7.90 (2H, d), 7.94 (2H, d), 8.30 (1H, s).

MS (EIpos): m/z=523 (M+H)⁺

### Example 28

### N-(4-{[5,6-Bis(4-methoxyphenyl)furo[2,3-d]pyrimidin-4-yl]amino}transcyclohexyl)-N′-ethylurea

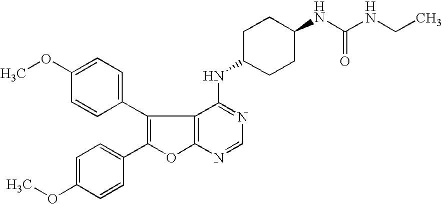

59.6 mg (0.13 mmol) of the compound from example 168 are added to a solution of 9.5 mg (0.13 mmol) of 1-isocyanatoethane in 3 ml of THF, and the reaction mixture is stirred at room temperature for 24 hours. 5 ml of ethanol are then added to the mixture, and the product, which is obtained as a precipitate, is filtered off with suction and washed with ethanol. This gives 40 mg (57.9%) of product as a colorless solid.

¹H-NMR (300 MHz, DMSO-d₆): δ=0.96 (3H, t), 1.01-1.28 (4H, m), 1.64-1.77 (2H, m), 1.82-1.94 (2H, m), 2.98 (2H, dq), 3.76 (3H, s), 3.87 (3H, s), 4.68 (1H, d), 5.58-5.69 (2H, m), 6.93 (2H, d), 7.16 (2H, d), 7.41 (2H, d), 7.46 (2H, d), 8.31 (1H, s).

MS (EIpos): m/z=516 (M+H)⁺

### Example 29

### N-Cycloheptyl-5,6-bis(4-methoxyphenyl)-N-methylfuro[2,3-d]pyrimidine-4-amine

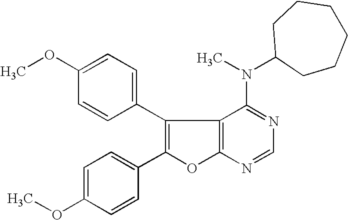

The cycloheptylmethylamine used for the synthesis was prepared analogously to the following literature procedure: K. A. Neidigh, M. A. Avery, J. S. Williamson, S. Bhattacharyya, J. Chem. Soc., Perkin Trans. 1 1998, 2527-2531.

398 mg (2.88 mmol) of potassium carbonate and 366 mg (2.88 mmol) of cycloheptylmethylamine are added to a solution of 176 mg (0.48 mmol) of the compound from example XV in 10 ml of THF, and the reaction mixture is stirred under reflux for 12 hours. Ethyl acetate is then added to the mixture, and the mixture is washed with saturated aqueous sodium bicarbonate solution. The organic phase is removed, dried over magnesium sulfate and concentrated under reduced pressure. The mixture is separated by prepative MPLC (Column: 100 g of silica gel, mobile phase: cyclohexane/ethyl acetate 10:1; flow rate 80 ml/min; UV detection at 210 nm). Concentration under reduced pressure gives 180 mg (82%) of the product as a colorless solid.

¹H-NMR (300 MHz, DMSO-d₆): δ=1.06-1.70 (12H, m), 2.47 (3H, s), 3.74 (3H, s), 3.83 (3H, s), 4.08-4.23 (1H, m), 6.91 (2H, d), 7.07 (2H, d), 7.27-7.36 (4H, m), 8.33 (1H, s).

MS (EIpos): m/z=458.3 (M+H)⁺

### Example 30

### rac-4-[cis-3,5-Dimethyl-1-piperazinyl]-5,6-bis(4-methoxyphenyl)furo[2,3-d]pyrimidine

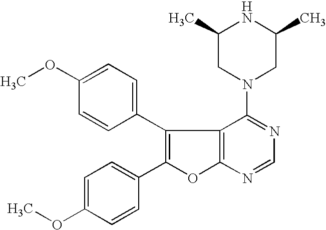

Analogously to the preparation procedure for example 29, 181 mg (1.31 mmol) of potassium carbonate and 100 mg (0.87 mmol) of cis-2,6-dimethylpiperazine are added to a solution of 80 mg (0.22 mmol) of the compound of example XV in 3 ml of THF, and the reaction mixture is stirred under reflux for 12 h. After work-up, the mixture is separated by preparative RP-HPLC (Column: YMC Gel ODS-AQ S-11 μm, 250×30 mm; mobile phase: acetonitrile/water; flow rate: 50 ml/min; UV detection at 210 nm). This gives 35 mg (36%) of the product as a colorless solid.

¹H-NMR (300 MHz, DMSO-d₆): δ=0.70 (6H, d), 2.09 (2H, dd), 2.30-2.59 (3H, m), 3.66 (2H, dd), 3.77 (3H, s), 3.82 (3H, s), 6.92 (2H, d), 7.08 (2H, d), 7.28-7.47 (4H, m), 8.48 (1H, s).

MS (EIpos): m/z=445.4 (M+H)⁺

### Example 31

### rac-1-[6-{4-[cis-3,5-Dimethyl-1-piperazinyl]phenyl}-5-(4-fluorophenyl)furo[2.3-d]-pyrimidin-4-yl]-4-piperidinol

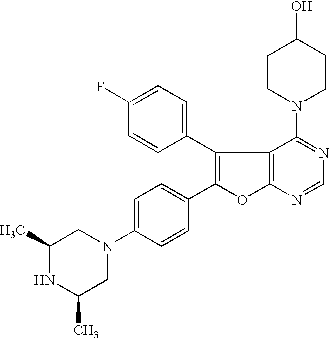

Analogously to the preparation procedure for example 14, 70 mg (0.22 mmol) of the compound of example 23, 4.1 mg (0.004 mmol) of tris(dibenzylideneacetone)dipalladium, 9.3 mg (0.015 mmol) of rac-2,2′-bis(diphenylphosphino)-1,1′-binaphthyl and 20.1 mg (0.18 mmol) of potassium tert-butoxide are initially charged in 6 ml of toluene. 51 mg (0.45 mmol) of cis-2,6-dimethylpiperazine are added, and the mixture is then stirred at 60° C. for 36 h. The reaction solution is diluted with ethyl acetate and washed with aqueous saturated sodium bicarbonate solution. The aqueous phase is extracted with dichloromethane and the organic phases are combined. Removal of the solvent under reduced pressure gives 42 mg (56%) of the product as a colorless solid.

¹H-NMR (300 MHz, DMSO-d₆): δ=0.92-1.49 (2H, m), 1.01 (6H, d), 1.37-1.49 (2H, m), 2.14 (2H, dd), 2.54-2.60 (1H, m), 2.70-2.92 (4H, m), 3.43-3.58 (3H m), 3.62 (2H, dd), 4.58 (1H, d), 6.87 (2H, d), 7.20 (2H, d), 7.29-7.50 (4H, m), 8.38 (1H, s).

MS (EIpos): m/z=502.3 (M+H)⁺

### Example 32

### N-{4-[4-(4-Methyl-1-piperazinyl)-5-phenylfuro[2,3-d]pyrimidin-6-yl]phenyl}-nicotinamide

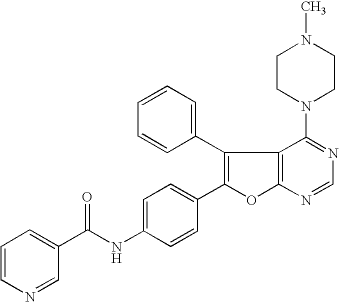

Analogously to the preparation of example 24, 2.0 mg (0.002 mmol) of tris-dibenzylideneacetone)dipalladium, 5.2 mg (0.009 mmol) of 9,9-dimethyl-4,5-bis(diphenylphosphino)xanthene, 32.6 mg (0.27 mmol) of nicotinamide and 102 mg (0.31 mmol) of cesium carbonate are initially charged in 0.25 ml of dry dioxane. Following addition of 100.0 mg (0.22 mmol) of the compound from example 22, the mixture is stirred at 100° C. for 30 h. After customary work-up and purification by preparative HPLC (Column: YMC Gel ODS-AQ S-11 μm, 250×30 mm; mobile phase: acetonitrile/water; flow rate: 50 ml/min; UV detection at 210 nm), 19 mg (17%) of the product are obtained in the form of a colorless solid.

¹H-NMR (300 MHz, DMSO-d₆): δ=1.94-2.08 (4H, m), 2.05 (3H, s), 3.12-3.23 (4H, m), 7.34-7.62 (8H, m), 7.75 (2H, d), 8.34-8.40 (1H, m), 8.44 (1H, s), 8.73-8.79 (1H, m), 9.08 (1H, s), 10.54 (1H, s).

MS (EIpos): m/z=491.4 (M+H)⁺

### Example 33

### N-{4-[5-(4-Fluorophenyl)-4-(4-methyl-1-piperazinyl)furo[2,3-d]pyrimidin-6-yl]-phenyl}-N-methylamine

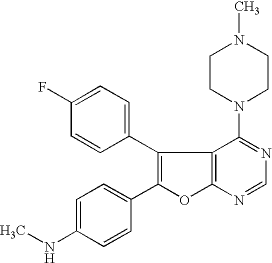

Analogously to the reaction of example 24, 100.0 mg (0.21 mmol) of the compound from example 21 are reacted with 15.2 mg (0.26 mmol) of N-methylformamide. However, the mixture is stirred at 100° C. for 40 h. After customary work-up, 39 mg (43%) of the decarbonylated amidation product are obtained in the form of a colorless solid.

¹H-NMR (300 MHz, DMSO-d₆): δ=1.93-2.08 (4H, m), 2.06 (3H, s), 2.67 (3H, d), 3.05-3.19 (4H, m), 6.10-6.18 (1H, m), 6.48 (2H, d), 7.13 (2H, d), 7.28-7.41 (2H, m), 7.41-7.51 (2H, m), 8.39 (1H, s).

MS (ESIpos): m/z=418.4 (M+H)⁺

The examples 34 to 184 listed in the table below are prepared analogously to the procedures given above:

| | | | | | | Synthesis |
|---|---|---|---|---|---|---|
| Exam- | Calculated | | Rt [min] | MS | m.p. | analogous |
| ple | mass | Structure | (method) | [M + H] | [° C.] | to example |
| 34 | 488.56 | 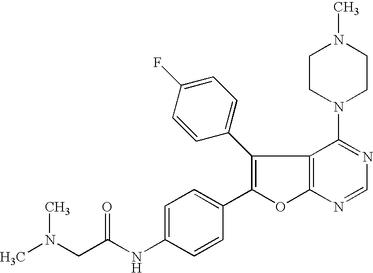 | 3.49 (A) | 489 | | 5 |
| 35 | 475.52 | 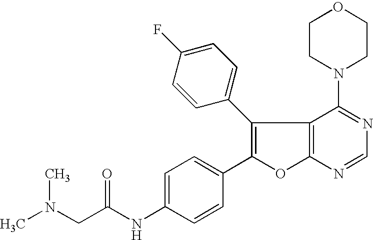 | | 475.9 | 228-235 | 5 |
| 36 | 527.02 | 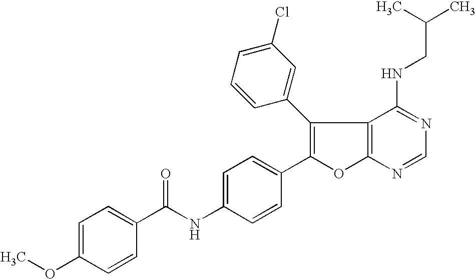 | 4.91 (A) | 526.9 | | 17 |
| 37 | 430.51 | 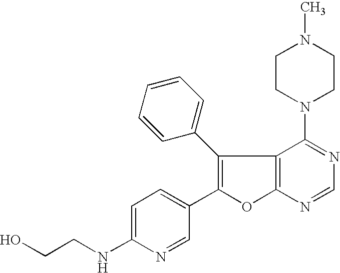 | | 431.2 | 219-221 | 7 |
| 38 | 470.53 | 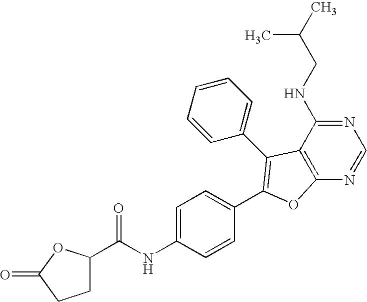 | 4.39 (A) | 471.2 | | 17 |
| 39 | 478.52 | 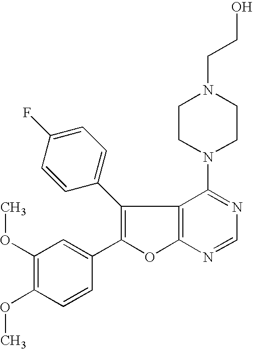 | | 478.9 | 156-158 | 2 |
| 40 | 429.52 | 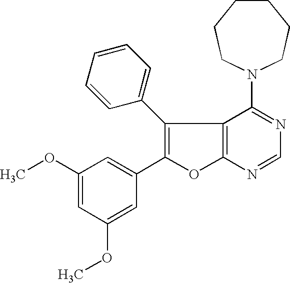 | 5.51 (B) | 430.4 | | 2 |
| 41 | 449.48 | 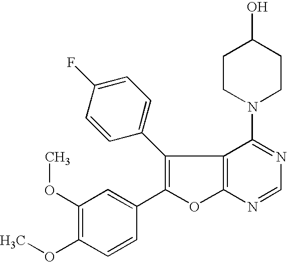 | 3.82 (B) | 450.4 | | 2 |
| 42 | 517.59 | 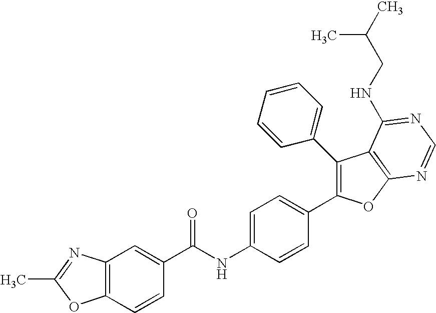 | 4.81 (B) | 518.4 | | 17* |
| 43 | 484.6 | 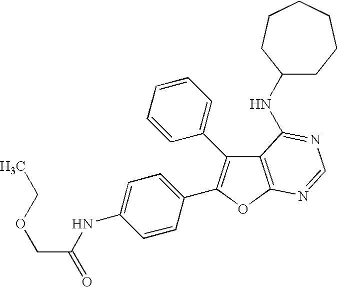 | 3.31 (E) | 485 | | 17 |
| 44 | 509.61 | 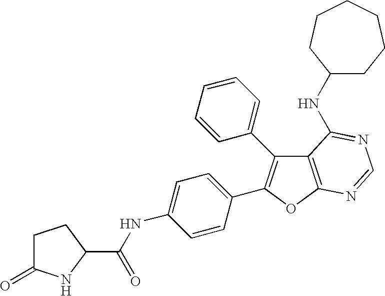 | 2.75 (E) | 510 | | 17 |
| 45 | 489.55 | 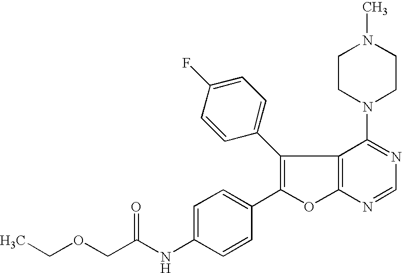 | 4.01 (A) | 490 | | 17 |
| 46 | 517.56 | 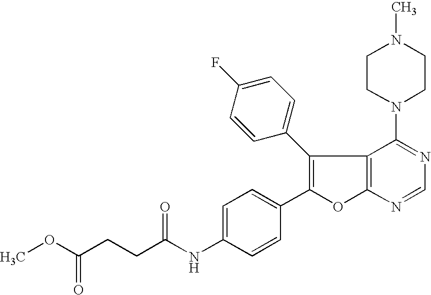 | | 518 | | 17 |
| 47 | 539.52 | 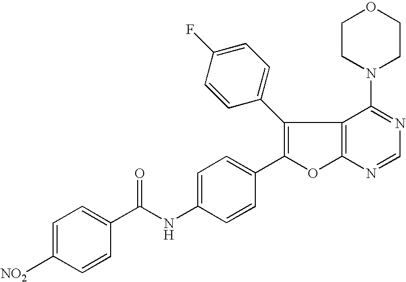 | | 540.1 | >250 | 17 |
| 48 | 552.56 | 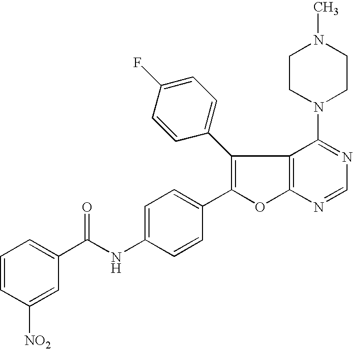 | | 552.9 | >250 | 17 |
| 49 | 501.56 | 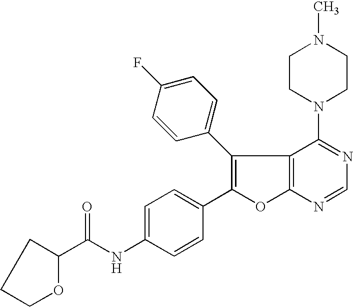 | | 502 | >250 | 17 |
| 50 | 492.58 | 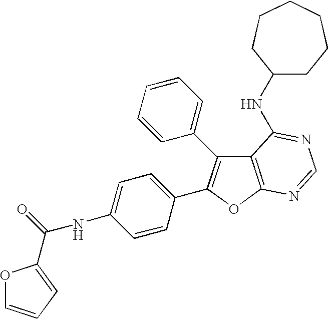 | 3.15 (E) | 493 | | 17 |
| 51 | 476.51 | 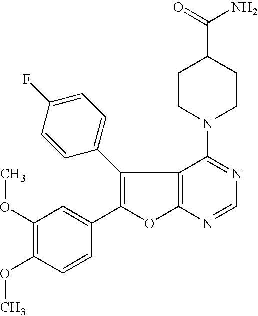 | | 476.9 | 235-237 | 2 |
| 52 | 539.52 | 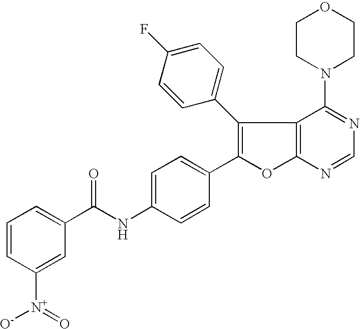 | 2.78 (E) | 540 | | 17 |
| 53 | 448.5 | 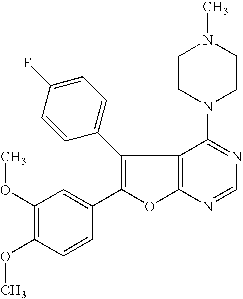 | | 449 | 168-169 | 2 |
| 54 | 537.59 | 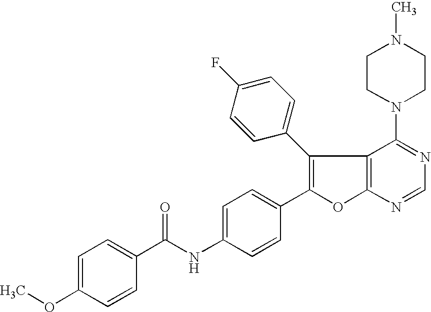 | | 538.1 | 252-253 | 17 |
| 55 | 503.53 | 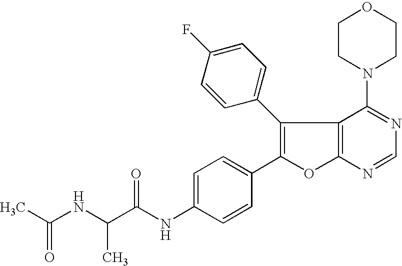 | | 504.2 | 250-252 | 17 |
| 56 | 459.52 | 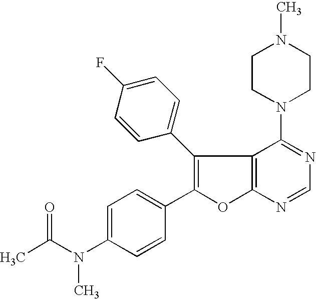 | 2.54 (B) | 460.4 | | 24 |
| 57 | 486.59 | 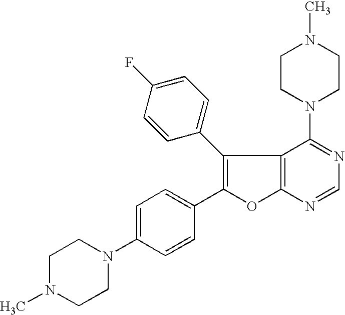 | 1.31 (E) | 487 | | 14 |
| 58 | 480.54 | 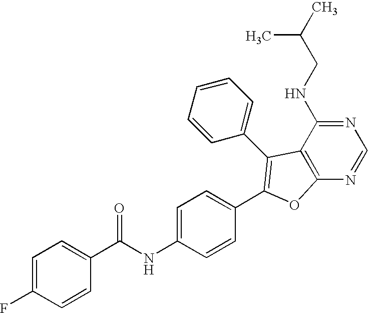 | 4.78 (A) | 481 | | 17 |
| 59 | 456.55 | 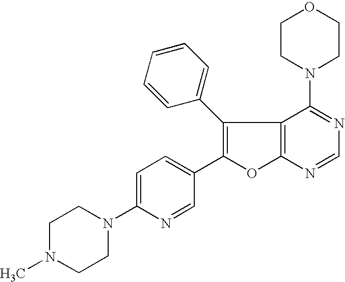 | 1.71 (E) | 457 | | 7 |
| 60 | 524.55 | 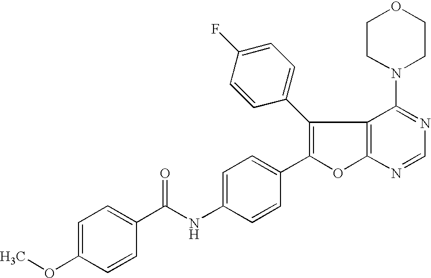 | 2.75 (E) | 525 | | 17 |
| 61 | 492.58 | 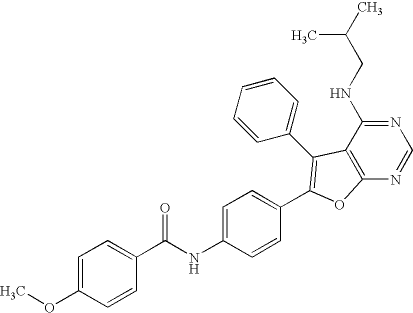 | 4.95 (B) | 492 | | 17 |
| 62 | 520.59 | 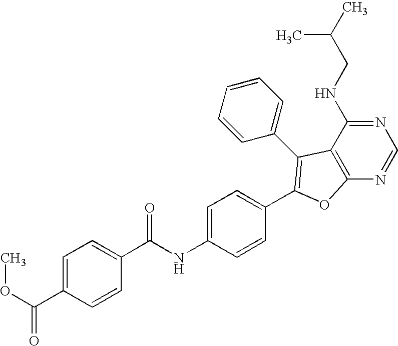 | 4.98 (A) | 520 | | 17 |
| 63 | 403.48 | 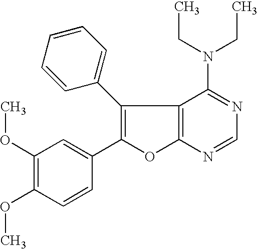 | | 404.3 | 125-126 | 2 |
| 64 | 476.55 | 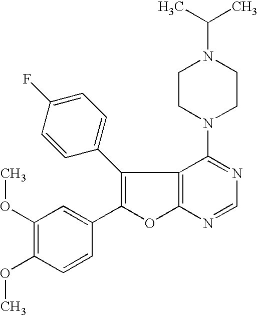 | | 477.6 | 180-181 | 2 |
| 65 | 491.52 | 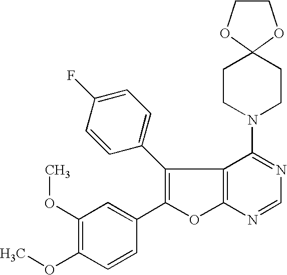 | 4.52 (B) | 492.4 | | 2 |
| 66 | 458.559 | 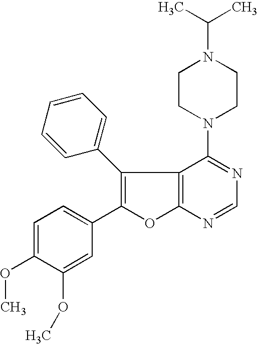 | 2.76 (B) | 459.4 | | 2 |
| 67 | 571.47 | 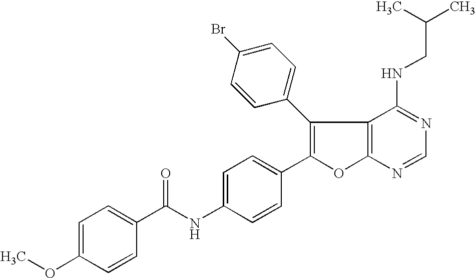 | 5.02 (A) | 572.7 | | 17 |
| 68 | 472.56 | 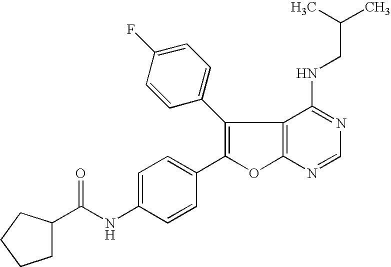 | 3.04 (E) | 473 | | 17 |
| 69 | 461.53 | 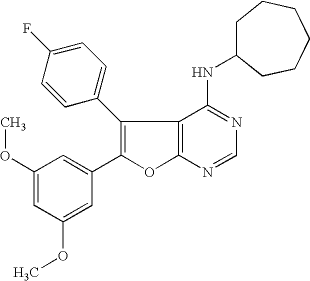 | | 462.1 | 170-172 | 1 |
| 70 | 508.55 | 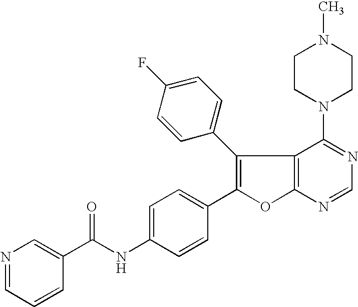 | | 509 | >250 | 17 |
| 71 | 488.52 | 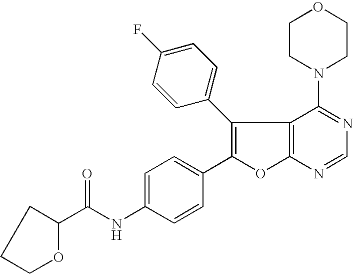 | | 489.3 | 201-202 | 17 |
| 72 | 600.61 | 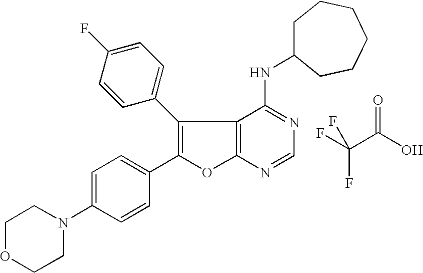 | 3.24 (E) | 487 (-TFA) | | 14 |
| 73 | 587.57 | 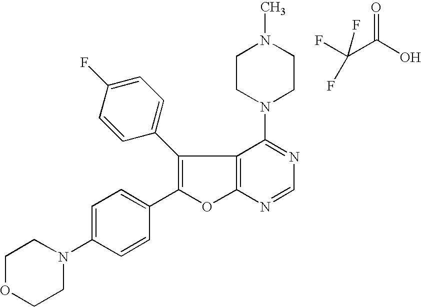 | 1.79 (E) | 474 (-TFA) | | 14 |
| 74 | 492.58 | 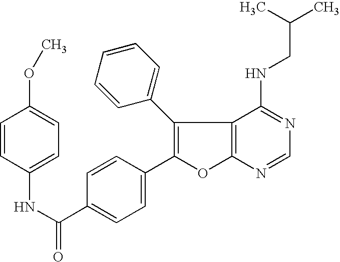 | 4.80 (A) | 493.1 | | 13 |
| 75 | 476.58 | 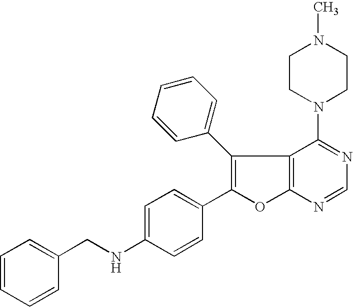 | | 477 | 161-163 | 7 |
| 76 | 545.68 | 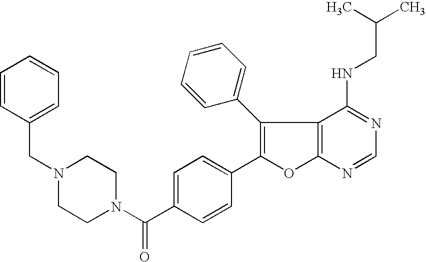 | 4.33 (A) | 546 | | 13 |
| 77 | 489.5 | 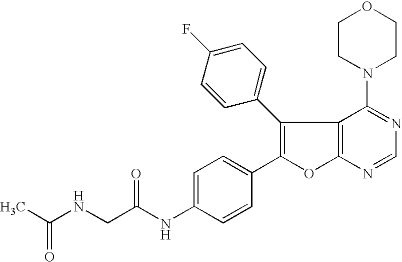 | 2.20 (E) | 490 | | 17 |
| 78 | 456.54 | 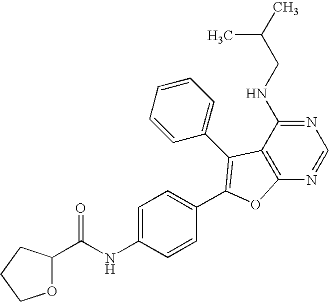 | 4.48 (A) | 457.2 | | 17 |
| 79 | 541.99 | 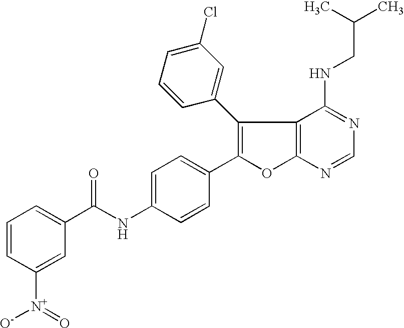 | 4.98 (A) | 542 | | 17 |
| 80 | 483.61 | 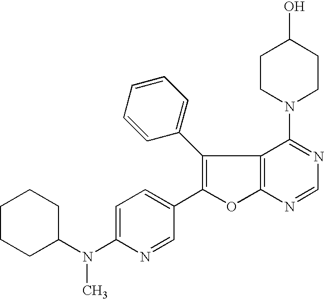 | 3.90 (B) | 484.4 | | 7 |
| 81 | 430.51 | 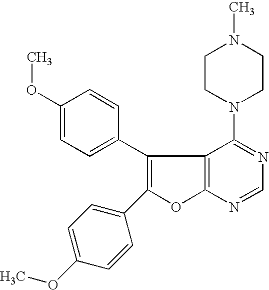 | 4.18 (A) | 431 | | 2 |
| 82 | 469.45 | 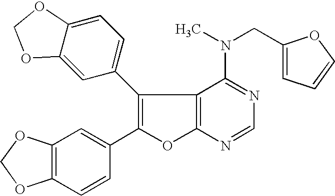 | 4.81 (B) | 470.4 | | 2 |
| 83 | 478.52 | 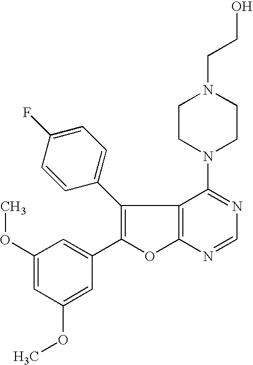 | | 479 | 161-163 | 2 |
| 84 | 462.52 | 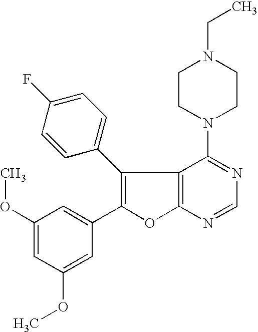 | | 463.1 | 146+147 | 2 |
| 85 | 483.61 | 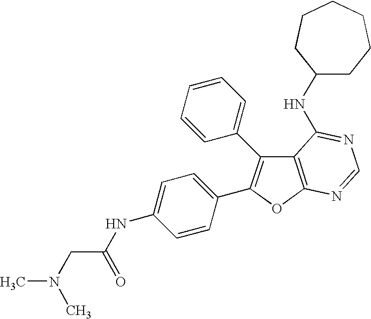 | 2.04 (E) | 484 | | 17 |
| 86 | 510.63 | 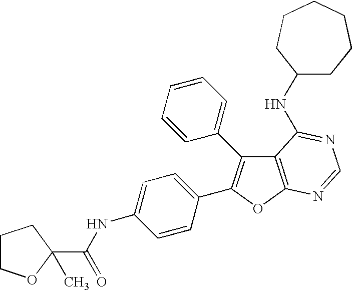 | 3.28 (E) | 511 | | 17* |
| 87 | 552.56 | 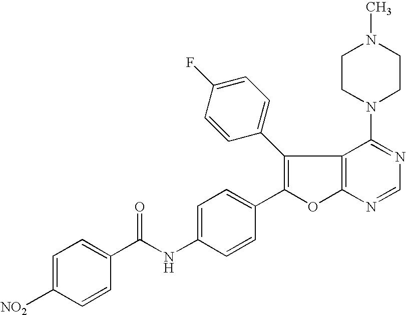 | 4.31 (A) | 552.8 | | 17 |
| 88 | 565.6 | 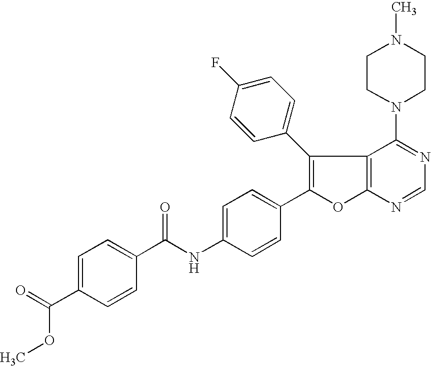 | | 566.1 | >250 | 17 |
| 89 | 473.57 | 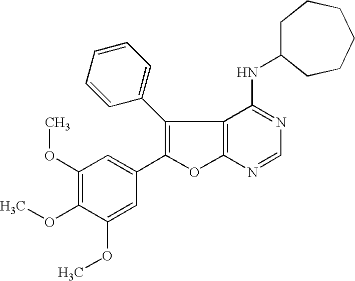 | 3.91 (E) | 474 | | 1 |
| 90 | 443.54 | 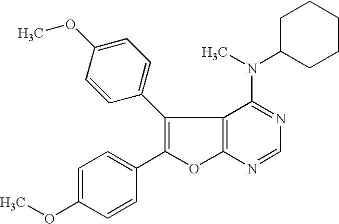 | 5.53 (B) | 444.4 | | 2 |
| 91 | 586.49 | 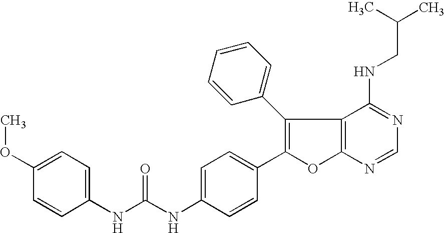 | 4.82 (A) | 585 | | 20 |
| 92 | 448.50 | 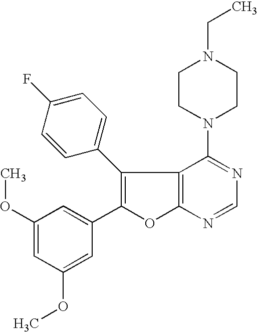 | | 449 | 151-155 | 2 |
| 93 | 476.55 | 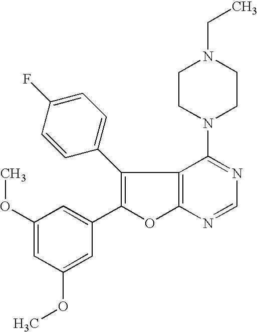 | | 477 | 151-152 | 2 |
| 94 | 405.89 | 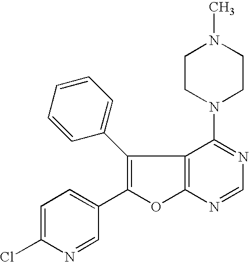 | | 406.1 | 173-175 | 2 |
| 95 | 464.53 | 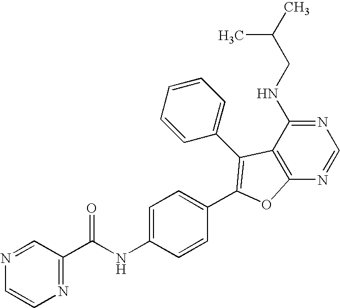 | 4.59 (A) | 464.9 | | 17 |
| 96 | 470.57 | 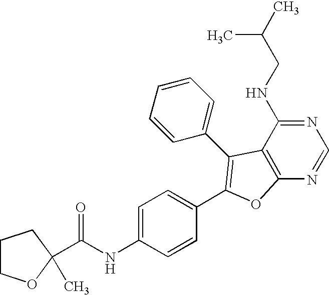 | 4.69 (A) | 471.1 | | 17* |
| 97 | 443.54 | 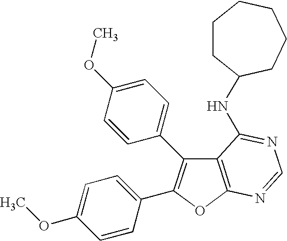 | 3.89 (E) | 444 | | 1 |
| 98 | 468.6 | 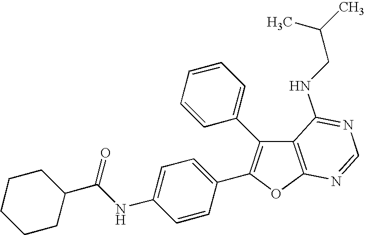 | 5.20 (B) | 469.3 | | 17 |
| 99 | 399.49 | 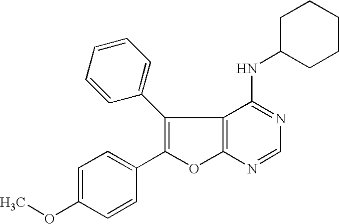 | 3.76 (B) | 400 | | 1 |
| 100 | 511.67 | 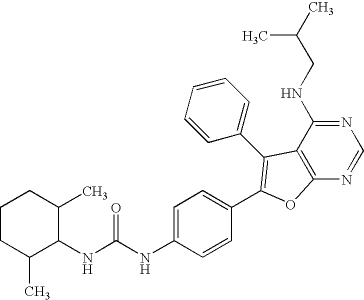 | 5.05 (A) | 512.5 | | 20* |
| 101 | 441.48 | 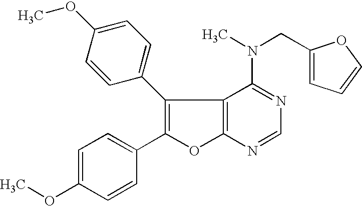 | 5.06 (D) | 442.1 | | 2 |
| 102 | 454.57 | 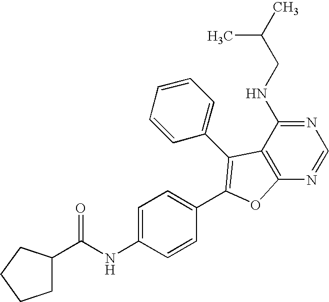 | 4.94 (A) | 454 | | 17 |
| 103 | 541.45 | 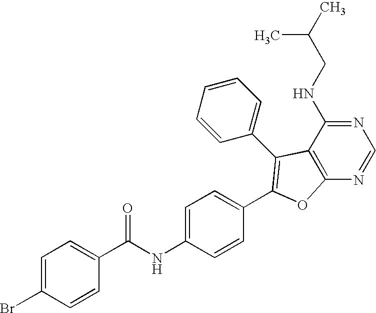 | 4.97 (A) | 541.1 | | 17 |
| 104 | 507.55 | 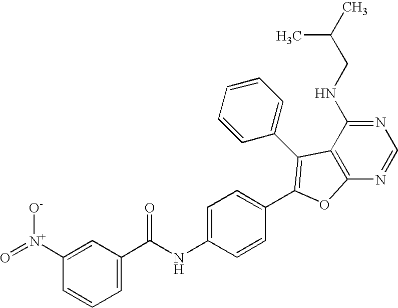 | 4.76 (A) | 508.1 | | 17 |
| 105 | 537.62 | 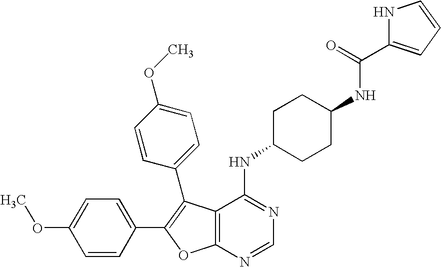 | 4.38 (B) | 538.4 | | 26 |
| 106 | 530.55 | 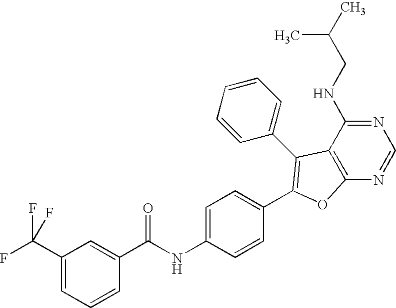 | 5.01 (A) | 531 | | 17 |
| 107 | 583.73 | 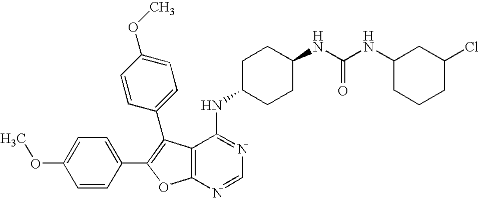 | 334 (E) | 584 | | 28* |
| 108 | 421.45 | 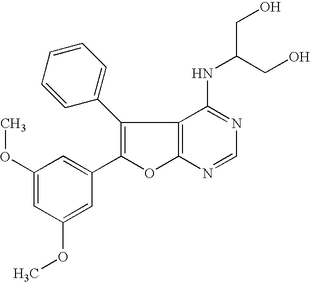 | | 422.1 | 131-133 | 1 |
| 109 | 459.54 | 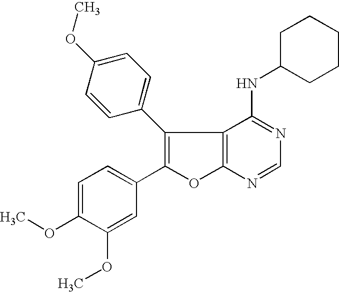 | | 460.2 | 181-182 | 1 |
| 110 | 440.54 | 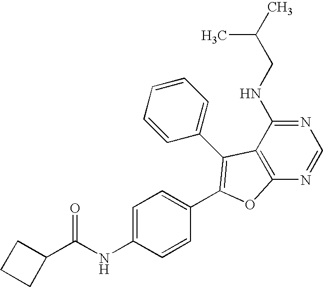 | 4.62 (A) | 440 | | 17 |
| 111 | 431.49 | 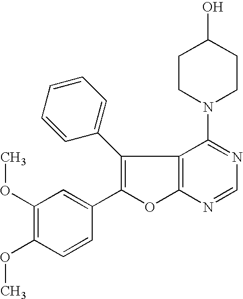 | | 432.3 | 176-178 | 2 |
| 112 | 460.53 | 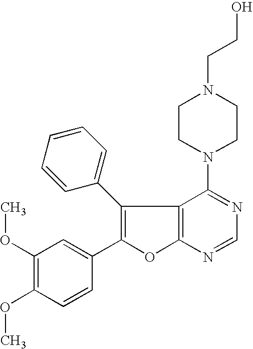 | | 461.1 | 188-189 | 2 |
| 113 | 430.51 | 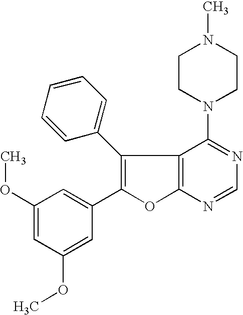 | | 431.3 | 156-157 | 2 |
| 114 | 511.55 | 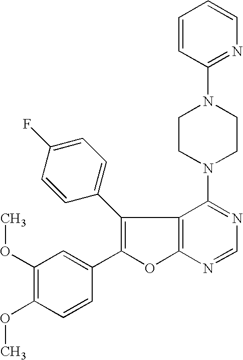 | | 512.2 | 210-212 | 2 |
| 115 | 461.53 | 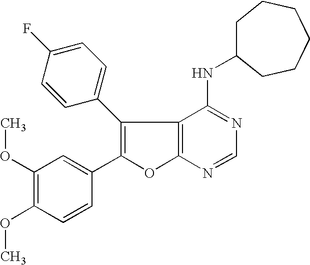 | | 462.1 | 180-182 | 2 |
| 116 | 435.45 | 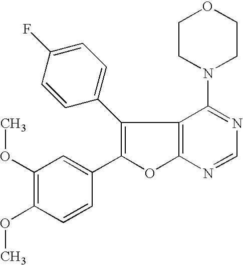 | | 436.1 | 211-213 | 2 |
| 117 | 460.53 | 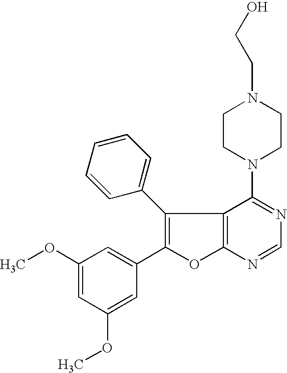 | 2.81 (B) | 461.9 | | 2 |
| 118 | 471.51 | 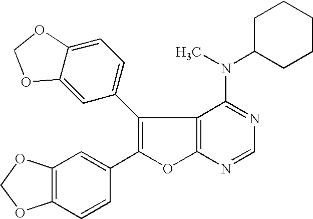 | 5.27 (B) | 472.3 | | 2 |
| 119 | 445.43 | 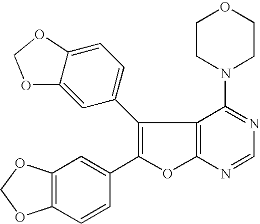 | 4.31 (B) | 446.3 | | 2 |
| 120 | 487.56 | 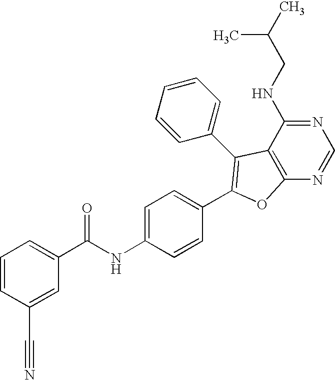 | 4.98 (A) | 488.1 | | 17 |
| 121 | 458.53 | 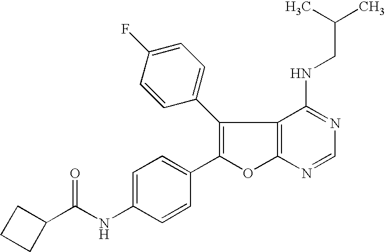 | 2.99 (E) | 459 | | 17 |
| 122 | 541.99 | 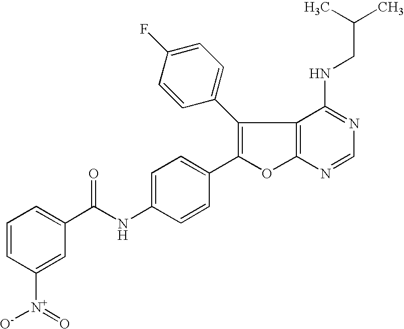 | 4.95 (A) | 542 | | 17 |
| 123 | 538.58 | 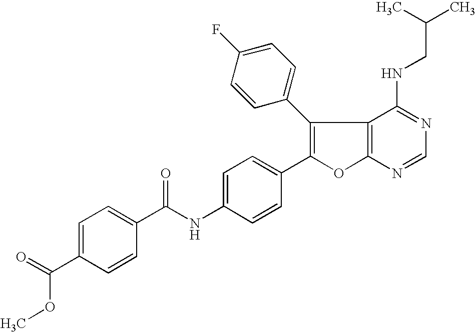 | 2.99 (E) | 539 | | 17 |
| 124 | 586.44 | 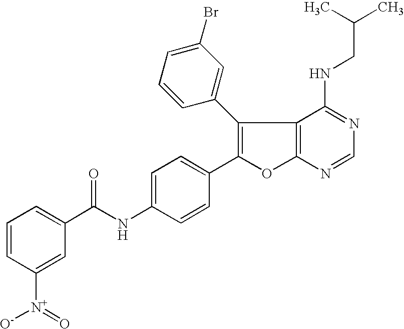 | 5.1 (A) | 587.7 | | 17 |
| 125 | 468.6 | 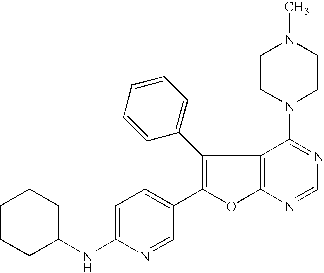 | | 469 | 208-209 | 7 |
| 126 | 517.67 | 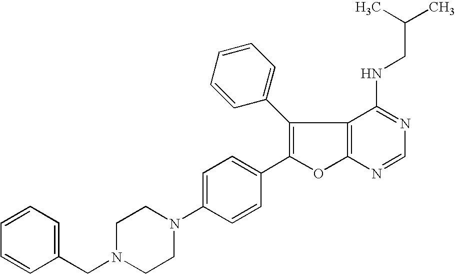 | 4.44 (A) | 518.1 | | 14 |
| 127 | 530.57 | 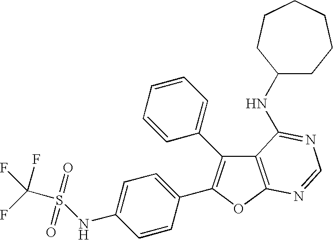 | 3.30 (E) | 531 | | 18 |
| 128 | 431.49 | 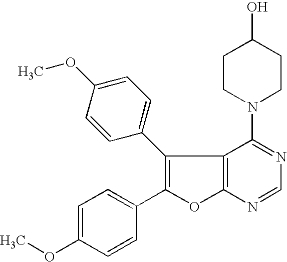 | 2.54 (E) | 432 | | 2 |
| 129 | 463.54 | 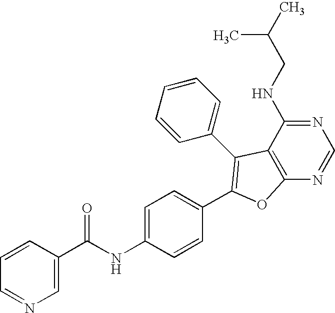 | 4.12 (A) | 464 | | 17 |
| 130 | 463.54 | 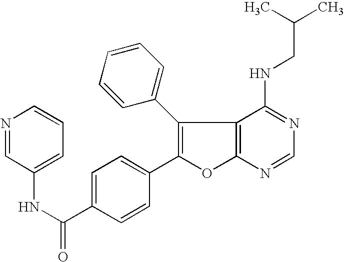 | 4.17 (A) | 464 | | 13 |
| 131 | 421.45 | 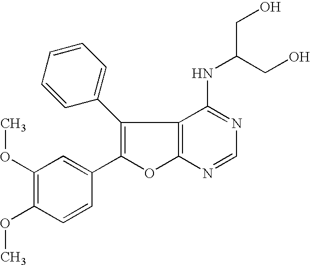 | 2.91 (E) | 422 | | 2 |
| 132 | 499.61 | 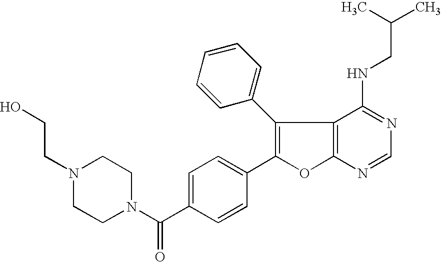 | 3.94 (A) | 500.1 | | 13 |
| 133 | 591.71 | 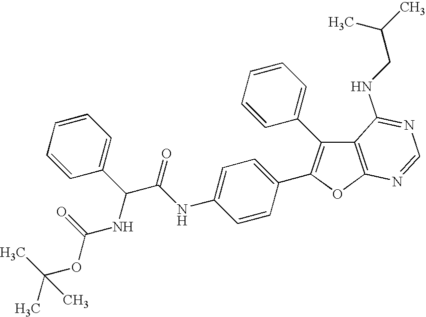 | 5.11 (A) | 592 | | 17 |
| 134 | 457.55 | 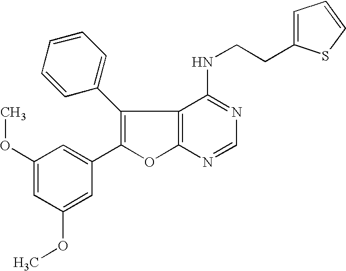 | | 458.1 | 152-153 | 1 |
| 135 | 415.49 | 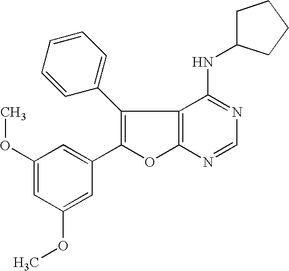 | | 416.2 | 153-154 | 1 |
| 136 | 473.57 | 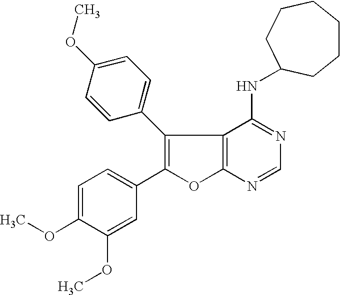 | 5.3 (B) | 474.4 | | 1 |
| 137 | 498.53 | 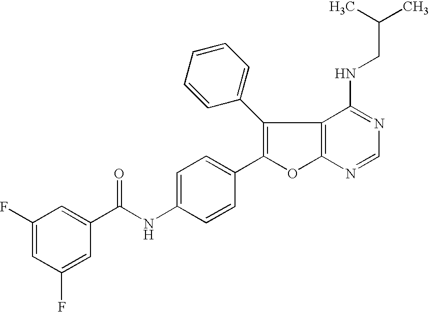 | 5.09 (A) | 499.2 | | 17 |
| 138 | 430.51 | 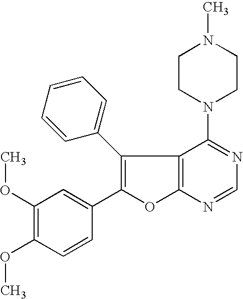 | 2.75 (B) | 431.4 | | 2 |
| 139 | 494.64 | 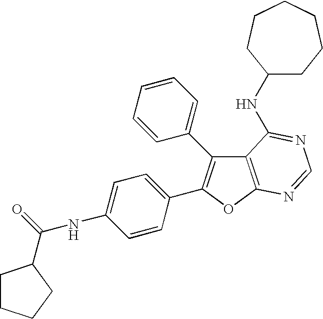 | 3.89 (E) | 495 | | 17 |
| 140 | 510.59 | 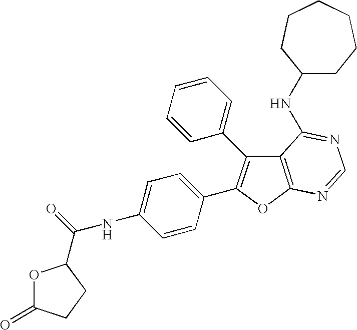 | 3.44 (E) | 511 | | 17 |
| 141 | 447.51 | 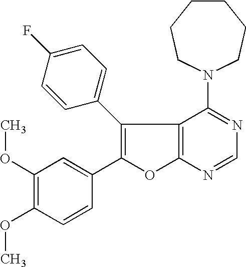 | | 448 | 148-150 | 2 |
| 142 | 466.97 | 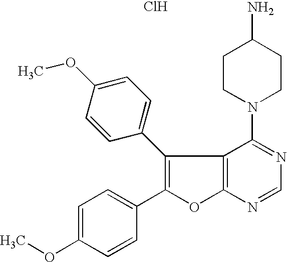 | 3.00 (B) | 431.2 (—HCl) | | 2* |
| 143 | 644.59 | 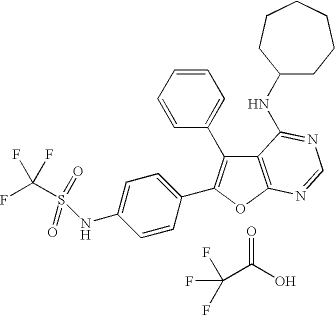 | 3.88 (E) | 531 (-TFA) | | 18 |
| 144 | 429.517 | 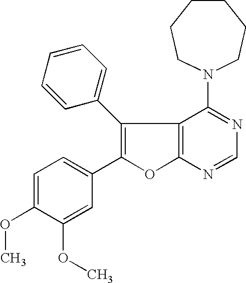 | 5.24 (B) | 430.6 | | 2 |
| 145 | 458.47 | 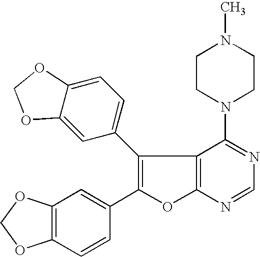 | 1.75 (E) | 459.3 | | 2 |
| 146 | 506.56 | 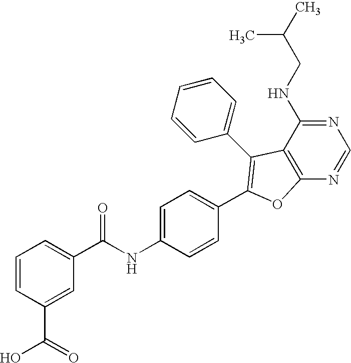 | 4.50 (A) | 507 | | 17 |
| 147 | 522.6 | 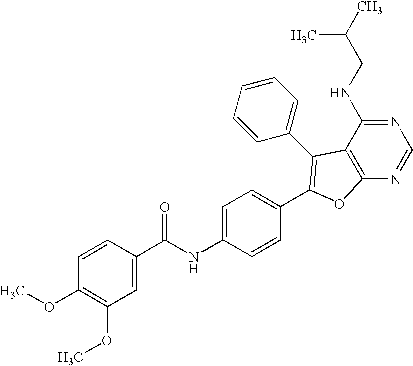 | 4.64 (A) | 523.1 | | 17 |
| 148 | 525.54 | 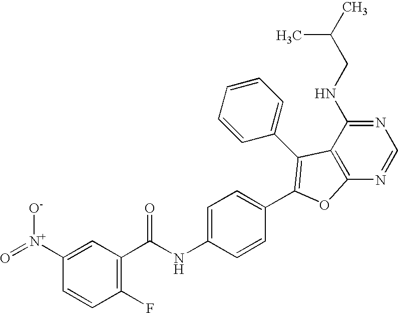 | 4.87 (A) | 526 | | 17 |
| 149 | 419.47 | 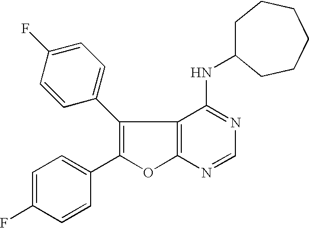 | 3.42 (E) | 420 | | 2 |
| 150 | 406.43 | 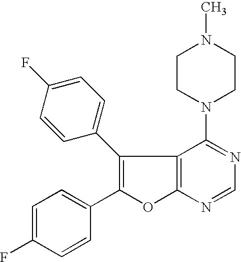 | 1.86 (E) | 407 | | 2 |
| 151 | 434.49 | 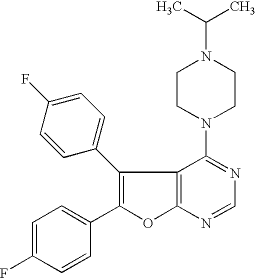 | 1.90 (E) | 435 | | 2 |
| 152 | 463.54 | 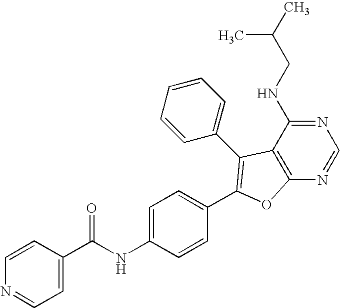 | 4.12 (A) | 464 | | 17 |
| 153 | 457.53 | 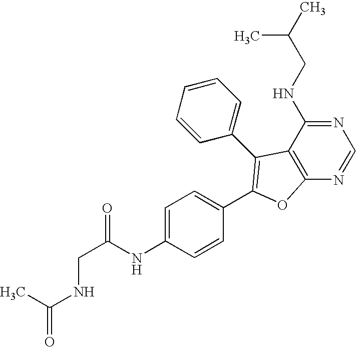 | 4.02 (A) | 458.2 | | 17 |
| 154 | 458.52 | 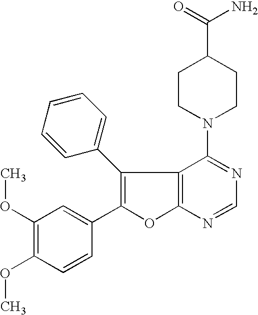 | | 459.2 | >250 | 2 |
| 155 | 470.57 | 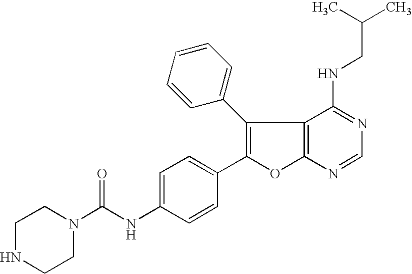 | 3.98 (A) | 471.2 | | 20* |
| 156 | 417.51 | 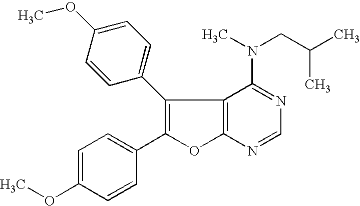 | 5.21 (B) | 418.7 | | 2 |
| 157 | 405.45 | 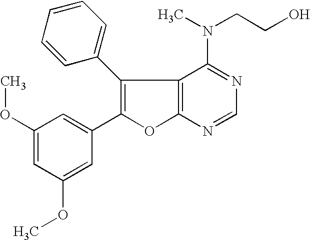 | | 406.2 | 132-134 | 2 |
| 158 | 471.51 | 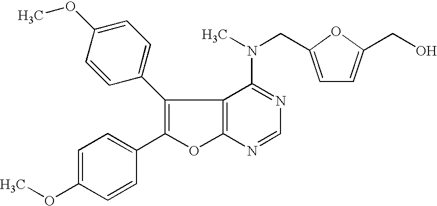 | 4.34 (B) | 472.2 | | 2* |
| 159 | 435.48 | 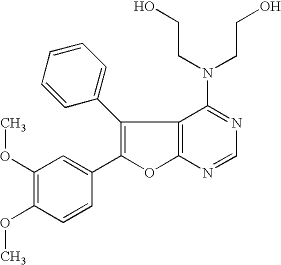 | | 436.2 | 137-139 | 2 |
| 160 | 463.38 | 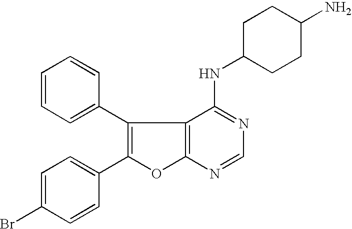 | 4.38 (A) | 463 | | Intermediate product XIa |
| 161 | 469.59 | 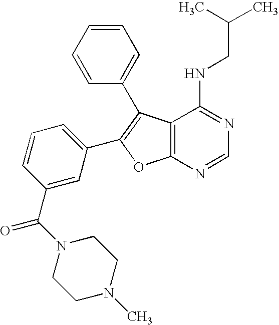 | 4.02 (A) | 470.2 | | 13 |
| 162 | 417.41 | 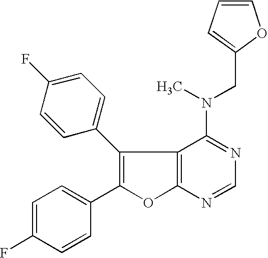 | 5.24 (B) | 418.3 | | 2 |
| 163 | 455.56 | 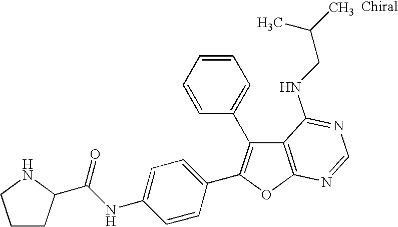 | 4.13 (A) | 456 | | 17* |
| 164 | 491.59 | 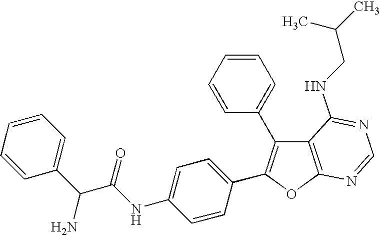 | 4.26 (A) | 492 | | 17* |
| 165 | 403.48 | 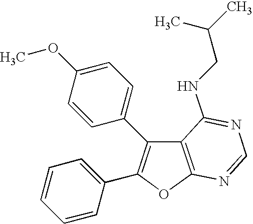 | 4.76 (A) | 404.18 | | Intermediate XVII |
| 166 | 375.43 | 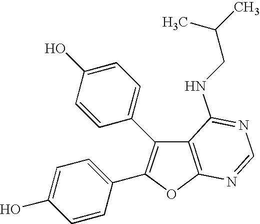 | 3.20 (E) | 376.25 | | 165 with subsequent cleavage with HBr* |
| 167 | 430.51 | 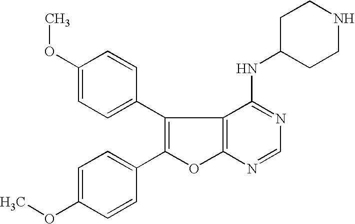 | 4.19 (A) | 431.3 | | Intermediate XVII* |
| 168 | 444.53 | 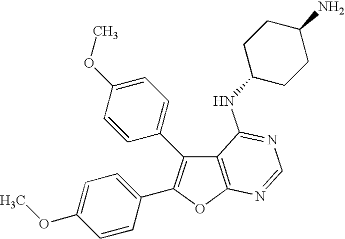 | 3.01 (B) | 445.3 | | 1 |
| 169 | 494.57 | 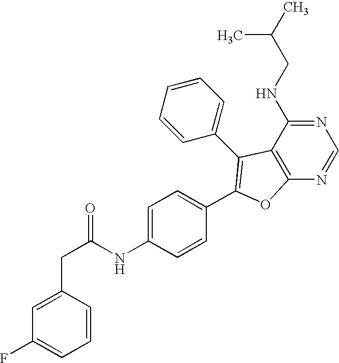 | 4.75 (A) | 495.1 | | 17 |
| 170 | 420.51 | 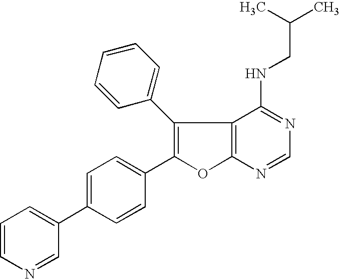 | 4.16 (A) | 421 | | Intermediate XIa with subsequent Stille coupling* |
| 171 | 460.53 | 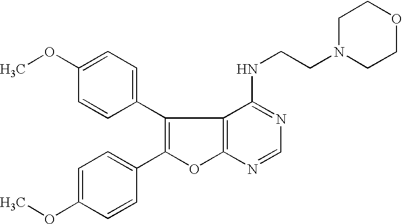 | 4.21 (A) | 461 | | 1 |
| 172 | 424.46 | 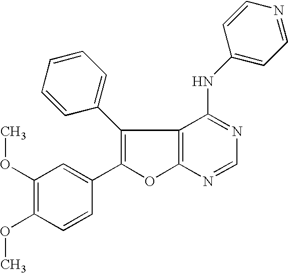 | | 425 | >250 | 2 |
| 173 | 469.59 | 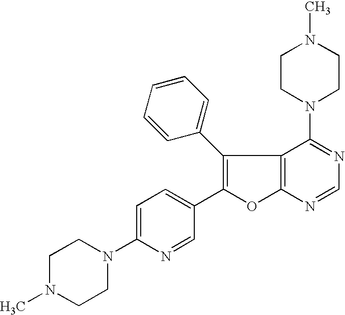 | 1.34 (E) | 470 | 176-177 | 7 |
| 174 | 427.51 | 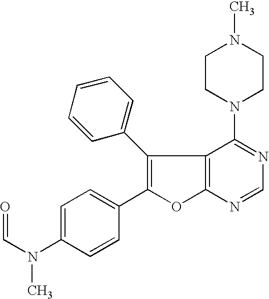 | 2.50 (B) | 428.3 | | 24 |
| 175 | 428.44 | 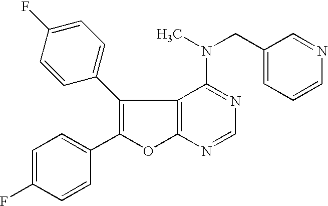 | 2.24 (E) | 429 | | 2 |
| 176 | 434.49 | 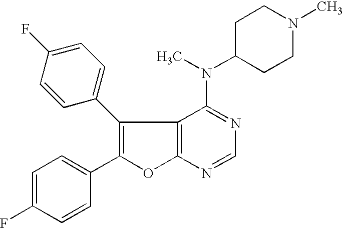 | | 435 | 180-181 | 2 |
| 177 | 457.48 | 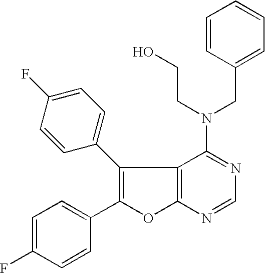 | | 458.2 | 156-158 | 2 |
| 178 | 498.63 | 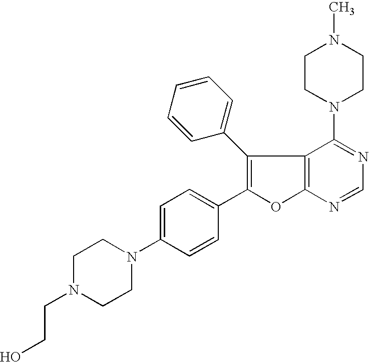 | 1.32 (E) | 499 | | 14 |
| 179 | 496.66 | 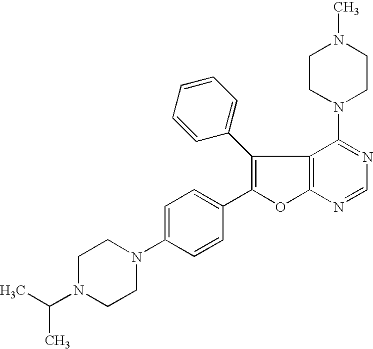 | 1.42 (E) | 497 | | 14 |
| 180 | 483.93 | 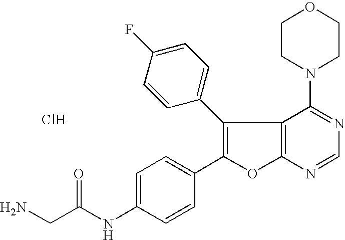 | 2.38 (A) | 447.4 (—HCl) | | 6 |
| 181 | 510.01 | 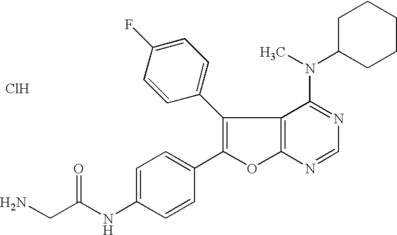 | 2.10 (E) | 473 (—HCl) | | 6 |
| 812 | 473.55 | 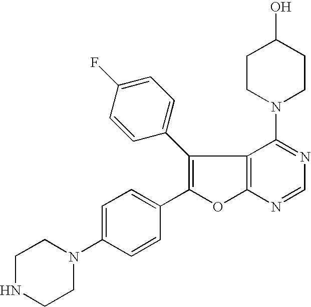 | 2.16 (B) | 474.3 | | 14 |
| 183 | 517.6 | 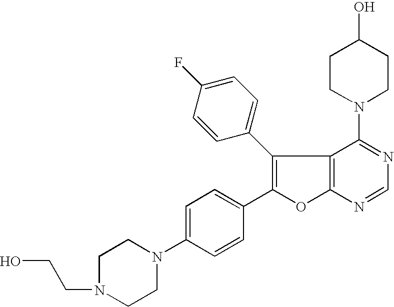 | 2.60 (B) | 518.3 | | 14 |
| 184 | 501.6 | 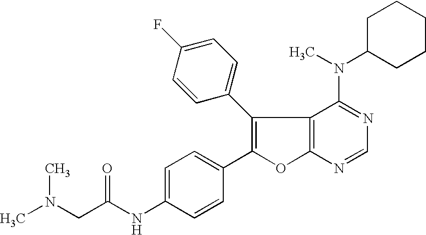 | 3.3 (B) | 501 | | 17 |

### Example 185

### 1-[6-(4-Bromophenyl)-5-phenylfuro[2,3-d]pyrimidin-4-yl]-4-piperidinol

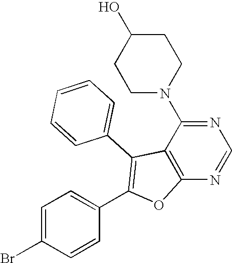

Analogously to the reaction of example 21, 3.00 g (7.78 mmol) of the compound of example XII are reacted with 3.15 g (31.12 mmol) of 4-hydroxypiperidine. This gives 3.12 g (89%) of the product as a white solid.

¹H-NMR (300 MHz, DMSO-d₆): δ=0.96-1.10 (m, 2H), 1.33-1.45 (m, 2H), 2.81-2.93 (m, 2H), 3.43-3.57 (m, 3H), 4.56 (d, 1H), 7.26-7.33 (m, 2H), 7.38-7.47 (m, 2H), 7.49-7.59 (m, 5H), 8.42 (s, 1H)

MS (ESIpos): m/z=450 (M+H)⁺

### Example 186

### 1-(6-{4-[(3-Chlorophenyl)amino]phenyl}-5-phenylfuro[2,3-d]pyrimidin-4-yl)-4-piperidinol

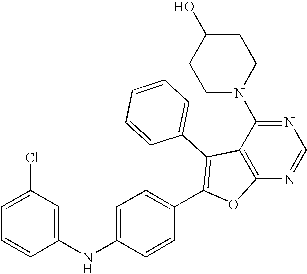

85.0 mg (0.67 mmol) of 3-chloroaniline are added to a reddish suspension of 6.1 mg (0.01 mmol) of tris(dibenzylideneacetone)dipalladium (0), 11.1 mg (0.02 mmol) of rac-2,2′-bis(diphenylphoshino)-1,1′-binaphthyl, 100.0 mg (0.22 mmol) of the compound of example 185 and 29.9 mg (0.27 mmol) of potassium tert-butoxide in 2 ml of toluene. The reaction mixture is heated at 60° C. for 24 h, with vigorous stirring. After cooling, dichloromethane (about 5 ml) is added to the reaction mixture, and the mixture is filtered and concentrated under reduced pressure. The mixture is separated by preparative RP-HPLC (Column: YMC Gel ODS-AQ S-11 μm, 250×30 mm; mobile phase: acetonitrile/water; flow rate: 50 ml/min; UV detection at 210 nm). This gives, after concentration under reduced pressure, 36 mg (33%) of the product as a colorless solid.

¹H-NMR (300 MHz, DMSO-d₆): δ=0.95-1.09 (m, 2H), 1.32-1.45 (m, 2H), 2.79-2.91 (m, 2H), 3.41-3.56 (m, 3H), 4.54 (d, 1H), 6.86-6.92 (m, 1H), 6.98-7.09 (m, 4H), 7.21-7.33 (m, 2H), 7.40-7.58 (m, 5H), 8.39 (s, 1H), 8.63 (s, 1H)

MS (ESIpos): m/z=497.4 (M+H)⁺

### Example 187

### tert-Butyl 1-[6-(4-bromophenyl)-5-(4-fluorophenyl)furo[2,3-d]pyrimidin-4-yl]-4-piperidinylcarbamate

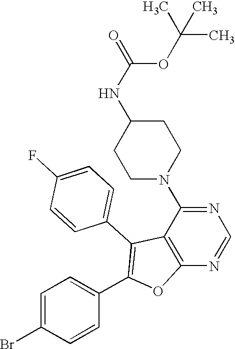

Analogously to the reaction of example 21, 1 g (2.59 mmol) of the compound of example XII are reacted with 2.08 g (10.37 mmol) of tert-butyl 4-piperidinylcarbamate. This gives 1.4 g (98%) of the product as a white solid.

1H-NMR (300 MHz, CDCl₃): δ=0.93-1.08 (m, 2H), 1.41 (s, 9H), 1.58-1.69 (m, 2H), 2.68-2.81 (m, 2H), 3.37-3.52 (m, 1H), 4.28 (br s, 1H), 7.27-7.50 (m, 8H), 8.47 (s, 1H)

MS (ESIpos): m/z=550 (M+H)⁺

### Example 188

### tert-Butyl 1-(5-(4-fluorophenyl)-6-{4-[formyl(methyl)amino]phenyl}furo[2,3-d]-pyrimidin-4-yl)-4-piperidinylcarbamate

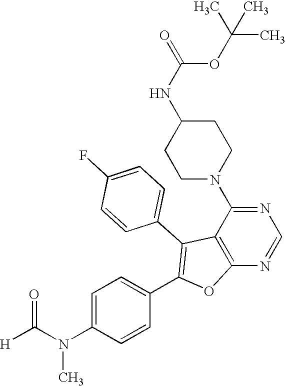

Analogously to the preparation of example 24, 3.33 mg (0.003 mmol) of tris(dibenzylideneacetone)dipalladium, 8.42 mg (0.01 mmol) of 9,9-dimethyl-4,5-bis(diphenylphoshino)xanthene, 12.9 mg (0.22 mmol) of N-methylformamide and 71.16mg (0.22 mmol) of cesium carbonate are initially charged in 1 ml of dry dioxane. After addition of 100.0 mg (0.18 mmol) of the compound from example 187, the mixture is stirred at 100° C. for 18 h. Customary work-up and purification by preparative RP-HPLC (Column: YMC Gel ODS-AQ S-11 μm, 250×30 mm; mobile phase: acetonitrile/water; flow rate: 50 ml/min; UV detection at 210 nm) gives 41 mg (39%) of the product in the form of a yellow solid.

MS (ESIpos): m/z=528 (M+H)⁺

### Example 189

### 4-[4-(4-Amino-1-piperidinyl)-5-(4-fluorophenyl)furo[2,3-d]pyrimidin-6-yl]-phenyl(methyl)formamide Hydrochloride

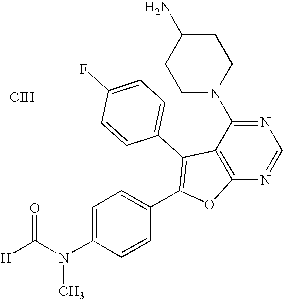

0.43 ml (1.71 mmol) of a 4 M solution of HCl in dioxane is added to a solution of 30 mg (0.06 mmol) of the compound from example 188 in 0.5 ml of dioxane, and the clear solution is stirred at room temperature for 4 h. A white solid is formed. The solvent is removed under reduced pressure, giving 25 g (88%) of product in the form of a colorless solid.

¹H-NMR (300 MHz, DMSO-d₆): δ=1.09-1.48 (m, 4H), 1.55-1.67 (m, 2H), 2.63-2.77 (m, 2H), 2.99-3.14 (m, 1H), 3.18 (s, 3H), 3.72-3.83 (m, 2H), 7.3-7.61 (m, 7H), 7.87-7.99 (m, 2H), 8.47 (s, 1H), 8.62 (s, 1H)

MS (ESIpos): m/z=428 (M+H)⁺

### Example 190

### 1-[6-(2′-Nitro-1,1′-biphenyl-4-yl)-5-phenylfuro[2,3-d]pyrimidin-4-yl]-4-piperidinol

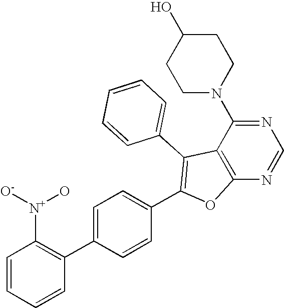

A suspension of 23.38 mg (0.03 mmol) of bis(triphenylphosphine)palladium(II) chloride, 150 mg (0.33 mmol) of the compound from example 185 and 72.28 mg (0.43 mmol) of (2-nitrophenyl)boric acid in 4 ml of N,N-dimethylformamide is stirred at 70° C. for 1 h. 0.5 ml of a 2 M aqueous sodium carbonate solution are then added to the yellow solution, and the reaction mixture is stirred at room temperature overnight. The crude reaction mixture is separated by preparative RP-HPLC (Column: YMC Gel ODS-AQ S-11 μm, 250×30 mm; mobile phase: acetonitrile/water; flow rate: 50 ml/min; UV detection at 210 nm). This gives 40 mg (24%) of the product in the form of a colorless solid.

¹H-NMR (300 MHz, DMSO-d₆): δ=0.95-1.10 (m, 2H), 1.33-1.46 (m, 2H), 2.82-2.96 (m, 2H), 3.44-3.58 (m, 3H), 7.31 (d, 1H), 7.42-7.80 (m, 10H), 7.98 (d, 1H), 8.44 (s, 1H)

MS (ESIpos): m/z=493.3 (M+H)⁺

### Example 191

### 5-(4-Fluorophenyl)-N-methyl-6-[6-(methylamino)-3-pyridinyl]furo[2,3-d]-pyrimidine-4-amine

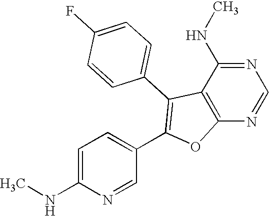

1.67 ml of a 33% strength solution of methylamine in ethanol are added to a solution of 200 mg (0.56 mmol) of the compound from example XIX in 1.67 ml of methanol. 0.08 ml (0.59 mmol) of triethylamine are added to the reaction mixture, and the mixture is then stirred at 70° C. in a closed vessel for 16 h. The crude mixture is separated directly by preparative RP-HPLC (Column: YMC Gel ODS-AQ S-11 μm, 250×30 mm; mobile phase: acetonitrile/water; flow rate: 50 ml/min; UV detection at 210 nm). This gives 38 mg (14%) of product in the form of a colorless solid.

¹H-NMR (300 MHz, CDCl₃): δ=2.93 (d, 3H), 2.98 (d, 3H), 4.51 (br d. 1H), 4.82 (br d, 1H), 6.35 (d, 1H), 7.19-7.29 (m, 2H), 7.41-7.49 (m, 2H), 7.50 (dd, 1H), 8.17 (d, 1H), 8.42 (s. 1H)

MS (DCI): m/z=350 (M+H)⁺

### Example 192

### 1-[6-(6-Chloro-3-pyridinyl)-5-(4-fluorophenyl)furo[2,3-d]pyrimidin-4-yl]-4-piperidinol

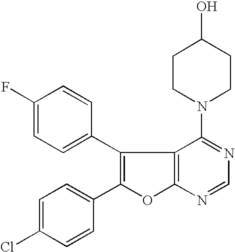

The compound is prepared in a yield of 70% from the compound of example XIX and 4-piperidinol, analogously to the procedure for the synthesis of example 9.

¹H-NMR (300 MHz, DMSO-d₆): δ=0.99-1.13 (m, 2H), 1.39-1.51 (m 2H), 2.88-2.98 (m, 2H), 3.48-3.59 (m, 3H), 4.60 (d, 1H), 7.36-7.45 (m, 2H), 7.48-7.59 (m, 3H), 7.77 (dd, 1H), 8.34 (d, 1H), 8.47 (s, 1H)

MS (ESIpos): m/z=425 (M+H)⁺

### Example 193

### 1-(5-(4-Fluorophenyl)-6-{6-[(2-hydroxyethyl)amino]-3-pyridinyl]furo[2,3-d]pyrimidin-4-yl)-4-piperidinol

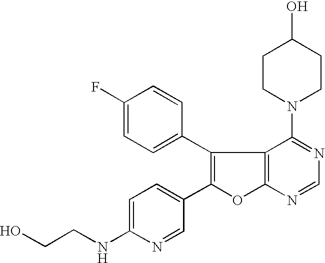

A mixture of 1000mg (2.35 mmol) of the compound from example 192 and 2-aminoethanol is stirred at 135° C. for 12 h. After cooling, the mixture is diluted with ethyl acetate and, after customary work-up, separated by preparative RP-HPLC (Column: YMC Gel ODS-AQ S-11 μm, 250×30 mm; mobile phase: acetonitrile/water; flow rate: 50 ml/min; UV detection at 210 nm). This gives 919 mg (87%) of the product in the form of a colorless solid.

¹H-NMR (300 MHz, DMSO-d₆): δ=0.97-1.11 (, 2H), 1.38-1.49 (m, 2H), 2.80-2.93 (m, 2H), 3.42-3.57 (m, 5H), 4.58 (br s, 2H), 6.49 (d, 1H), 6.98 (br s, 1H), 7.29-7.41 (m, 3H), 7.42-7.51 (m, 2H), 7.97 (d, 1H), 8.39 (s, 1H)

MS (ESIpos): m/z=450.4 (M+H)⁺

### Example 194

### 1-{5-(4-Fluorophenyl)-6-[6-(2-hydroxyethoxy)-3-pyridinyl]furo[2,3-d]pyrimidin-4-yl}-4-piperidinol

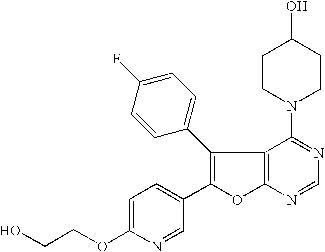

14.5 mg (0.26 mmol) of powdered potassium hydroxide are added to a solution of 100 mg (0.24 mmol) of the compound from example 192 in 1 ml of 1,2-ethanediol, and the reaction mixture is stirred at 100° C. for 2.5 h. After cooling, water (about 15 ml) is added and the resulting precipitate is filtered off with suction and dried under reduced pressure. The crude product is separated by preparative RP-HPLC (Column: YMC Gel ODS-AQ S-11 μm, 250×30 mm; mobile phase: acetonitrile/water; flow rate: 50 ml/min; UV detection at 210 nm). This gives 7 mg (6%) of product in the form of a colorless solid.

¹H-NMR (300 MHz, DMSO-d₆): δ=0.96-1.11 (m, 2H), 1.34-1.50 (m, 2H), 2.8-2.96 (m, 2H), 3.42-3.59 (m, 3H), 3.41-3.73 (m, 2H), 4.19-4.31 (m, 2H), 4.49 (d, 1H), 4.79 (t, 1H), 6.83 (d, 1H), 7.29-7.54 (m, 4H), 7.56-7.67 (m, 1H), 8.13 (d, 1H), 8.41 (s, 1H)

MS (ESIpos): m/z=451.3 (M+H)⁺

### Example 195

### 1-{5-(4-Fluorophenyl)-6-[6-(4-morpholinyl)-3-pyridinyl]furo[2,3-d]pyrimidin-4-yl}-4-piperidinol

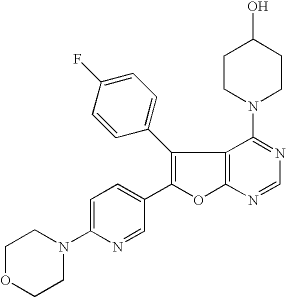

The compound is prepared analogously to the procedure for example 7 from example 192 and morpholine (yield: 85%).

¹H-NMR (300 MHz, CDCl₃): δ=0.97-1.11 (m, 2H), 1.38-1.49 (m, 2H), 2.81-2.93 (m, 2H), 3.44-3.57 (m, 7H), 3.62-3.71 (m, 4H), 4.58 (d, 1H), 6.82 (d, 1H), 7.31-7.41 (m, 2H), 7.43-7.54 (m, 3H), 8.12 (d, 1H), 8.39 (s, 1H)

MS (ESIpos): m/z=476.5 (M+H)⁺

### Example 196

### 1-{5-(4-Fluorophenyl)-6-[6-(4-morpholinyl)-3-pyridinyl]furo[2,3-d]pyrimidin-4-yl}-4-piperidinol Hydrochloride

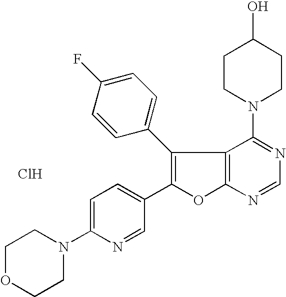

2 ml of a 4 M solution of HCl in dioxane are added to 40 mg (0.08 mmol) of the compound from example 195, and the reaction mixture is stirred at room temperature for 3 h. Removal of the solvent gives 43 mg (95%) of product in the form of a colorless solid.

MS (ESIpos): m/z=476.4 (M+H)⁺

### Example 197

### 4-(4-Isopropyl-1-piperazinyl)-5-phenyl-6-[4-(1-piperazinyl)phenyl]furo[2,3-d]pyrimidine Hydrochloride

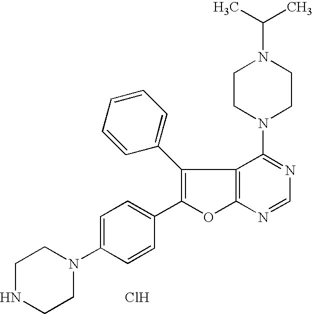

The bromide used, 6-(4-bromophenyl)-4-(4-isopropyl-1-piperazinyl)-5-phenylfuro[2,3-d]pyrimidine, is synthesized analogously to the preparation procedure of example 22 starting with the compound of example XII and 4-isopropylpiperazine.

Under protective gas and with vigorous exclusion of oxygen, 100 mg (0.21 mmol) of 6-(4-bromophenyl)-4-(4-isopropyl-1-piperazinyl)-5-phenylfuro[2,3-d]pyrimidine, 5.75 mg (0.01 mmol) of tris(dibenzylideneacetone)dipalladium, 13.0 mg (0.02 mmol) of rac-2,2′-bis(diphenylphosphino)-1,1′-binaphthyl and 28.2 mg (0.29 mmol) of sodium tert-butoxide are initially charged in 4 ml of toluene, and 54.1 mg (0.63 mmol) of piperazine are added. The reaction mixture is stirred at 60° C. for 20 h. After removal of the solvent, ethyl acetate is added and the mixture is worked up in the customary manner. The mixture is separated by preparative RP-HPLC (Column: YMC Gel ODS-AQ S-11 μm, 250×30 mm; mobile phase: acetonitrile/water+0.3% HCl; flow rate: 50 ml/min; UV detection at 210 nm). This gives 16 mg (18%) of product in the form of a yellow solid.

¹H-NMR (300 MHz, DMSO-d₆): δ=0.17 (d, 6H), 2.48-2.60 (m, 2H), 3.05-3.23 (m, 8H), 3.29-3.40 (m, 1H), 3.41-3.48 (m, 4H), 3.73 (d, 1H), 6.95 (d, 2H), 7.33 (d, 2H), 7.43-7.61 (m, 5H), 8.52 (s, 1H), 9.05 (br s, 1H), 10.51 (br s, 1H)

MS (ESIpos): m/z=477 (M+H)⁺

### Example 198

### 1-{5-[5-(4-Fluorophenyl)-4-(4-hydroxy-1-piperidinyl)furo[2,3-d]pyrimidin-6-yl]-2-pyridinyl}-4-piperidinol

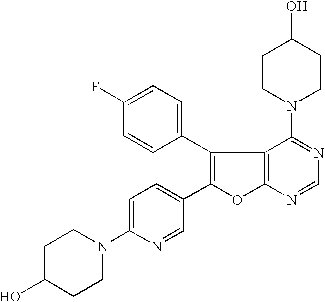

8.33 g (82.38 mmol) of 4-hydroxypiperidine are added to 593.4 mg (1.65 mmol) of the compound from example XIX. The reaction mixture is stirred at 135° C. for 14 h. After cooling, a solid is formed which is dissolved in water and ethanol. Following customary work-up, the mixture is separated by preparative RP-HPLC (Column: YMC Gel ODS-AQ S-11 μm, 250×30 mm; mobile phase: acetonitrile/water; flow rate: 50 ml/min; UV detection at 210 nm). This gives 380 mg (47%) of product in the form of a colorless solid.

¹H-NMR (200 MHz, DMSO-d₆): δ=1.18-1.57 (m, 4H), 1.66-1.86 (m, 2H), 2.77.-2.99 (m, 2H), 3.03-3.26 (m, 2H), 3.42-3.59 (m, 3H), 3.61-3.80 (m, 1H), 3.91-4.10 (m, 2H), 4.62 (d, 1H), 4.72 (d, 1H), 6.82 (d, 1H), 7.30-7.57 (m, 5H), 8.09 (d. 1H), 8.40 (s, 1H)

MS (ESIpos): m/z=490.4 (M+H)⁺

### Example 199

### 2-({5-[4-(1,4-Dioxa-8-azaspiro[4.5]dec-8-yl)-5-(4-fluorophenyl)furo[2,3-d]pyrimidin-6-yl]-2-pyridinyl}amino)ethanol

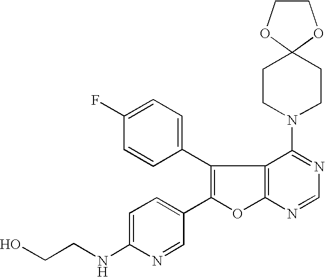

The chloride used, 8-[6-(6-chloro-3-pyridinyl)-5-(4-fluorophenyl)furo[2,3-d]pyrimidin-4-yl]-1,4-dioxa-8-azaspiro[4.5]decane, is synthesized analogously to the preparation procedure of example 22 starting with the compound of example XII and 1,4-dioxa-8-azaspiro[4.5]decane.

Analogously to the preparation procedure for example 7, 135 mg (0.29 mmol) of 8-[6-(6-chloro-3-pyridinyl)-5-(4-fluorophenyl)furo[2,3-d]pyrimidin-4-yl]-1,4-dioxa-8-azaspiro[4.5]decane and 3.04 g (49.70 mmol) of 2-aminoethanol are mixed and stirred at 135° C. for 14 h. Customary work-up and separation by preparation RP-HPLC (Column: YMC Gel ODS-AQ S-11 μm, 250×30 mm; mobile phase: acetonitrile/water; flow rate: 50 ml/min; UV detection at 210 nm) give 79mg (56%) of product in the form of a colorless solid.

¹H-NMR (200 MHz, DMSO-d₆): δ=1.23-1.38 (m, 4H), 3.15-3.27 (m, 4H), 3.27-3.33 (m, 2H), 3.41-3.55 (m, 2H), 3.80 (s, 4H), 4.70 (t, 1H), 6.48 (d, 1H), 7.02 (t, 1H), 7.28-7.52 (m, 5H), 7.98 (d, 1H), 8.41 (s, 1H)

MS (ESIpos): m/z=492.3 (M+H)⁺

### Example 200

### 1-{5-(4-Fluorophenyl)-6-[6-(4-isopropyl-1-piperazinyl-2-pyridinyl]furo[2,3-d]-pyrimidin-4-yl}-4-piperidinol

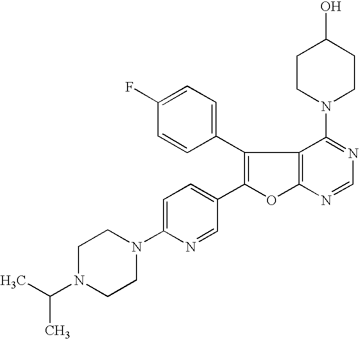

Analogously to the preparation procedure for example 7, 60 mg (0.14 mmol) of the compound from example 192 and 905.36 mg (7.06 mmol) of 4-isopropylpiperazine are mixed and stirred at 135° C. for 16 h. Following customary work-up, a precipitate is generated by addition of DMSO, and this precipitate is filtered off with suction, washed with DMSO and dried under reduced pressure. This gives 12 mg (16%) of product in the form of a colorless solid.

¹H-NMR (300 MHz, DMSO-d₆): δ=0.98 (d, 6H), 1.01-1.12 (m, 2H), 1.38-1.50 (m, 2H), 2.23-2.30 (m, 1H), 2.60-2.75 (m, 3H), 2.81-2.92 (m, 2H), 3.44-3.56 (m, 8H), 4.58 (d, 1H), 6.80 (d, 1H), 7.31-7.52 (m, 5H), 8.10 (d, 1H), 8.39 (s, 1H)

MS (ESIpos): m/z=517.4 (M+H)⁺

### Example 201

### N-{4-[5-Fluorophenyl)-4-(4-hydroxy-1-piperidinyl)furo[2.3-d]pyrimidin-6-yl]-phenyl}-L-prolinamide Hydrochloride

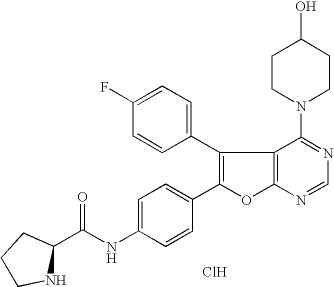

Analogously to the preparation procedure for example, 17, 60 mg (0.15 mmol) of the compound from example 23 and 38.32 mg (0.18 mmol) of 1-(tert-butoxycarbonyl)-L-prolin are reacted. Subsequent deprotection with a solution of HCl in dioxane gives 23 mg (13%) of product in the form of a colorless solid.

¹H-NMR (300 MHz, DMSO-d₆): δ=0.96-1.12 (m, 2H), 1.38-1.50 (m, 2H), 1.87-2.04 (m, 3H), 2.39-2.46 (m, 1H), 2.80-2.93 (m, 2H), 3.18-3.32 (m, 2H), 3.50-3.60 (m, 4H), 4.29-4.41 (m, 1H), 7.31-7.41 (m, 4H), 7.41-7.51 (m, 2H), 7.60 (d, 2H), 8.40 (s, 1H), 8.59-8.72 (m, 1H), 9.50-9.67 (m, 1H), 10.85 (s, 1H)

MS (ESIpos): m/z=502.2 (M+H)⁺

### Example 202

### N-(1-{5-[4-(4-Amino-1-piperidinyl)-5-(4-fluorophenyl)furo[2,3-d]pyrimidin-6-yl]-2-pyridinyl}-4-piperidinyl)acetamide Hydrochloride

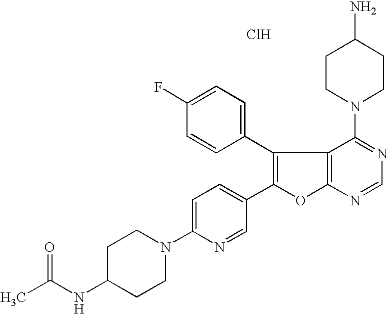

The chloride used, tert-butyl 1-[6-(6-chloro-3-pyridinyl)-5-(4-fluorophenyl)furo[2,3-d]pyrimidin-4-yl]-4-piperidinylcarbamate, is synthesized analogously to the preparation procedure of example 22 starting with the compound of example XII and tert-butyl 4-piperidinylcarbamate.

Analogously to the preparation procedure for example 7, 70 mg (0.11 mmol) of tert-butyl 1-[6-(6-chloro-2-pyridinyl)-5-(4-fluorophenyl)furo[2,3-d]pyrimidin-4-yl]-4-piperidinylcarbamate and 434.21 mg (3.05 mmol) of N-acetylpiperidine are mixed and stirred at 135° C. for 14 h. Customary work-up and separation by preparative RP-HPLC (Column: YMC Gel ODS-AQ S-11 μm, 250×30 mm; mobile phase: acetonitrile/water; flow rate: 50 ml/min; UV detection at 210 nm) gives 90 mg (75%) of tert-butyl 1-[6-{6-[4-(acetylamino)-1-piperidinyl]-3-pyridinyl}-5-(4-fluorophenyl)furo[2,3-d]pyrimidin-4-yl]-4-piperidinylcarbamate in the form of a colorless solid. A solution of HCl in dioxane is then added in the customary manner to 70 mg (0.11 mmol) of this substance, giving 62.5 mg (96%) of the corresponding hydrochloride in the form of a colorless solid.

¹H-NMR (400 MHz, D₂O): δ=0.95-1.08 (m, 2H), 1.28-1.41 (m, 2H), 1.57 (d, 2H), 1.77 (s, 3H)), 1.78-1.88 (m, 2H), 2.52-2.64 (m, 2H), 2.96-3.18 (m, 4H), 3.51-3.61 (m, 1H), 3.69-3.87 (m. 4H), 6.82 (d, 1H), 7.12 (dd. 2H), 7.21-7.29 (m, 2H), 7.40-7.46 (m, 1H), 7.70 (d, 1H), 8.12 (s, 1H)

MS (ESIpos): m/z=530.3 (M+H)⁺

### Example 203

### N-{5-[4-(4-Hydroxy-1-piperindinyl)-5-phenylfuro[2,3-d]pyrimidin-6-yl]-2-pyridinyl}acetamide

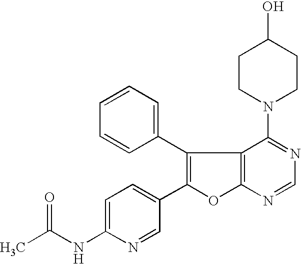

The chloride used, 1-[6-(6-Chloro-3-pyridinyl)-5-phenylfuro[2,3-d]pyrimidin-4-yl]-4-piperidinol, is synthesized analogously to the preparation procedure of example 192 starting with the compound of example XX and 4-hydroxypiperidine.

200 mg (0.49 mmol) of 1-[6-(6-chloro-3-pyridinyl)-5-phenylfuro[2,3-d]pyrimidin-4-yl]-4-piperidinol, 5.8 g (98.19 mmol) of acetamide and 1.69 g (12.23 mmol) of potassium carbonate are mixed and stirred at 210° C. for 32 h. The mixture is cooled to 70° C., and water is added. Following dilution with ethyl acetate, the phases are separated and the aqueous phase is extracted with ethyl acetate. The combined organic phases are washed with water, dried over magnesium sulfate and concentrated under reduced pressure. Separation by preparative RP-HPLC (Column: YMC Gel ODS-AQ S-11 μm, 250×30 mm; mobile phase: acetonitrile/water; flow rate: 50 ml/min; UV detection at 210 nm) gives 12 mg (6%) of product in the form of a colorless solid.

¹H-NMR (400 MHz, D₂O): δ=0.97-1.11 (m, 2H), 1.33-1.46 (m, 2H), 2.08 (s, 3H), 2.83-2.95 (m, 2H), 3.45-3.59 (m, 3H), 4.56 (d, 1H), 7.41-7.59 (m, 5H), 7.72 (dd, 1H) 8.02 (d, 1H), 8.20 (d, 1H), 8.42 (s, 1H)

MS (ESIpos): m/z=430.3 (M+H)⁺

### Example 204

### 1-[6-{6-[(2-Aminoethyl)amino]-3-pyridinyl}-5-(4-fluorophenyl)furo[2,3-d]pyrimidin-4-yl]-4-piperidinol Hydrochloride

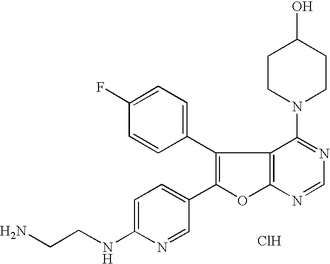

150 mg (0.35 mmol) of the compound from example 192 are suspended in 1.06 g (17.65 mmol) of ethylenediamine and stirred at 120° C. for 12 hours. After cooling, the reaction mixture is taken up in N,N-dimethylformamide and separated directly by preparative RP-HPLC (Column: YMC Gel ODS-AQ S-11 μm, 250×30 mm; mobile phase: acetonitrile/water+0.3% HCl; flow rate: 50 ml/min; UV detection at 210 nm). This gives 67 mg (39%) of product in the form of a colorless solid.

¹H-NMR (400 MHz, D₂O): δ=0.97-1.12 (m, 2H), 1.38-1.50 (m, 2H), 2.82-3.07 (m, 4H), 3.45-3.62 (m, 5H), 6.75 (d, 1H), 7.31-7.57 (m, 5H), 7.91-8.12 (br m, 2H), 7.96 (d, 1H), 8.41 (s, 1H)

MS (ESIpos): m/z=449.3 (M+H)⁺

Examples 205 to 331 listed in the table below are prepared analogously to the procedures given above:

| | | | | | Synthesis |
|---|---|---|---|---|---|
| Exam- | Calculated | | Rₜ [min] | MS | analogous |
| ple | mass | Structure | (method) | [M +]H]⁺ | to example |
| 205 | 550.63 | 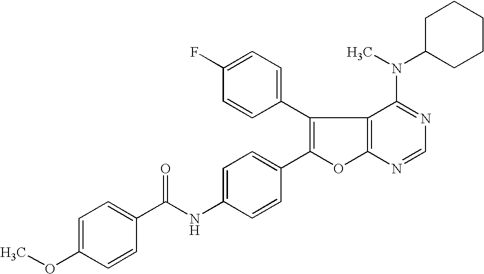 | 3.24 (E) | 551 | 5 |
| 206 | 428.49 | 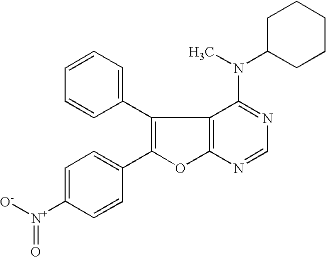 | 5.52 (A) | 429 | 3 |
| 207 | 387.44 | 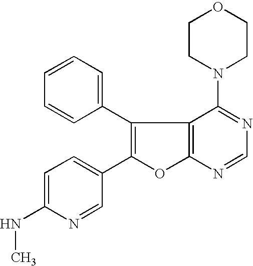 | 2.7 (B) | 388.3 | 33 |
| 208 | 433.44 | 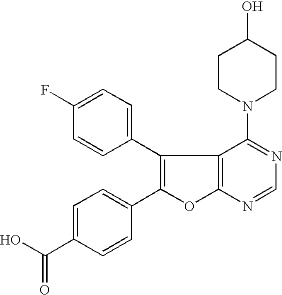 | 3.5 (B) | 434.3 | 12 |
| 209 | 501.56 | 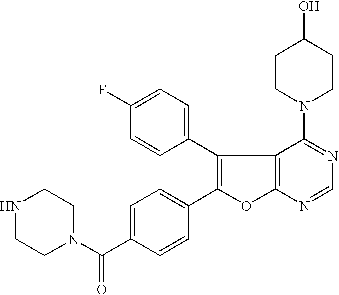 | 1.52 (E) | 502 | 13 |
| 210 | 430.46 | 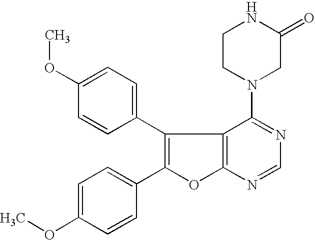 | 2.40 (E) | 431 | 2 |
| 211 | 463.53 | 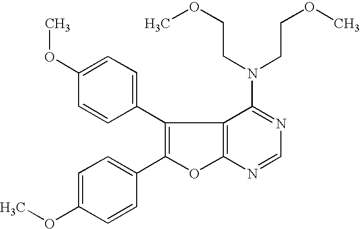 | 2.89 (E) | 464 | 2 |
| 212 | 529.66 | 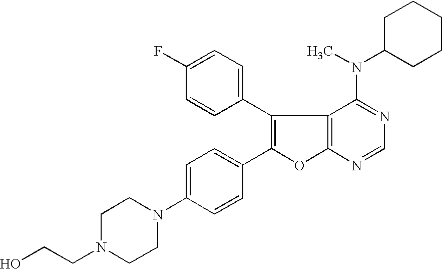 | 3.4 (B) | 530.2 | 14 |
| 213 | 541.67 | 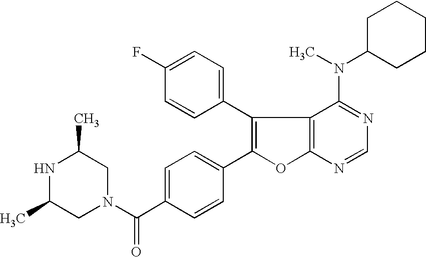 | 1.93 (E) | 542 | 13 |
| 214 | 557.67 | 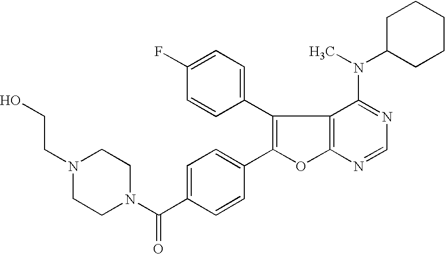 | 1.97 (E) | 558 | 13 |
| 215 | 446.55 | 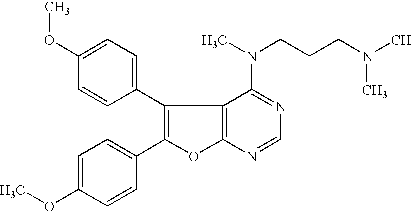 | 3.08 (B) | 447.3 | 2 |
| 216 | 445.49 | 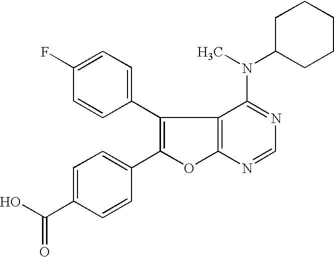 | 3.08 (E) | 446 | 12 |
| 217 | 444.53 | 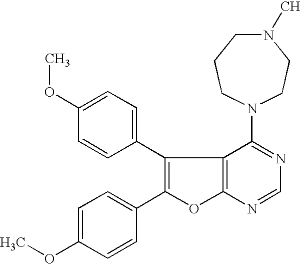 | 2.97 (B) | 445.2 | 2 |
| 218 | 452.51 | 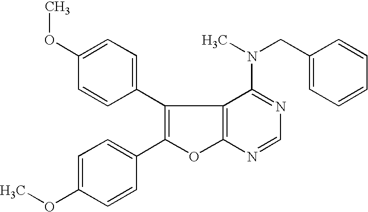 | 2.10 (E) | 453 | 2 |
| 219 | 497.96 | 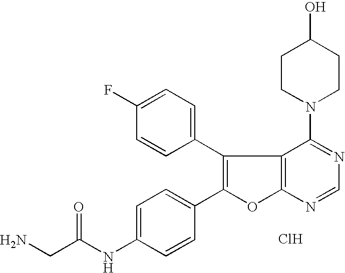 | 1.64 (E) | 462 | 5* |
| 220 | 431.49 | 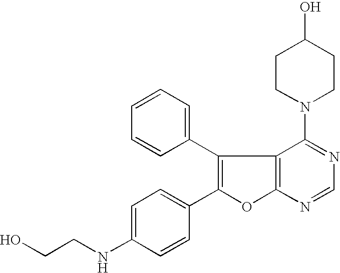 | 2.47 (B) | 432.3 | 7 |
| 221 | 499.63 | 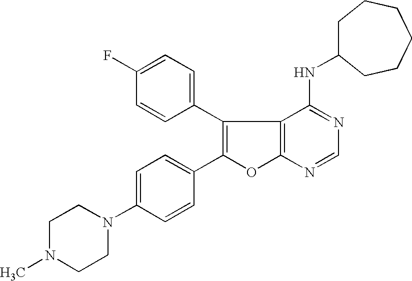 | 3.4 (B) | 500.47 | 14 |
| 222 | 387.44 | 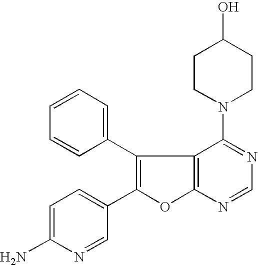 | 2.3 (B) | 388.31 | 8 |
| 223 | 455.56 | 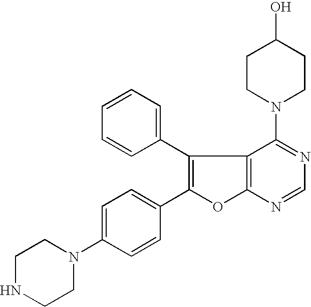 | 2.5 (B) | 456.4 | 14 |
| 224 | 405.43 | 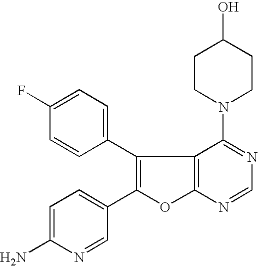 | 0.33 (B) | 406.3 | 8 |
| 225 | 386.45 | 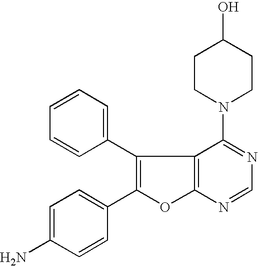 | 3.3 (B) | 387.4 | 25 |
| 226 | 449.31 | 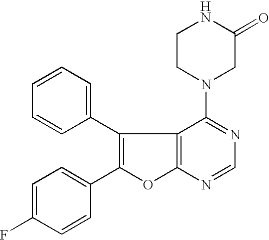 | 4.3 (B) | 449 | 22 |
| 227 | 513.61 | 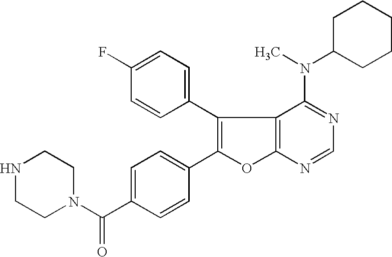 | 3.33 (A) | 514.3 | 13 |
| 228 | 419.46 | 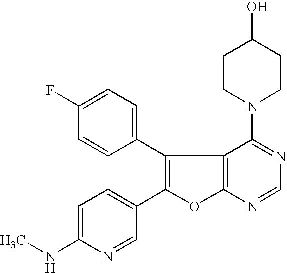 | 2.5 (B) | 420.4 | 33 |
| 229 | 485.58 | 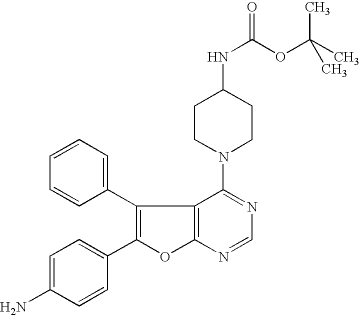 | 4.4 (B) | 486.4 | 25 |
| 230 | 515.44 | 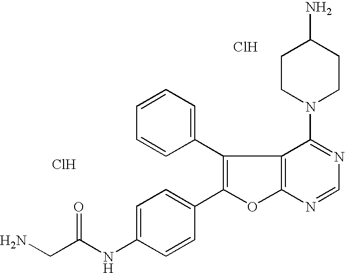 | 0.34 (B) | 443.4 | 5* |
| 231 | 458.39 | 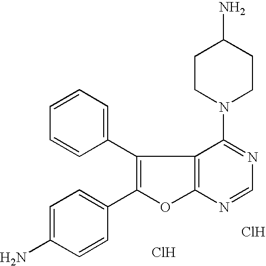 | 1.56 (E) | 386 | 25* |
| 232 | 473.53 | 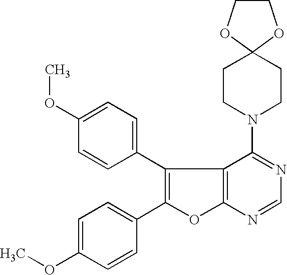 | 4.81 (B) | 474.4 | 2 |
| 233 | 339.45 | 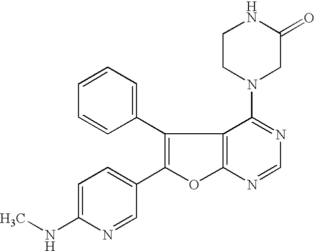 | 3.58 (B) | 400.4 | 33 |
| 234 | 429.47 | 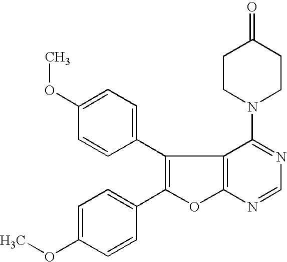 | 4.5 (B) | 430.4 | 2 |
| 235 | 415.45 | 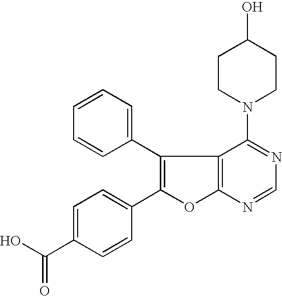 | 3.43 (B) | 416.3 | 12 |
| 236 | 371.44 | 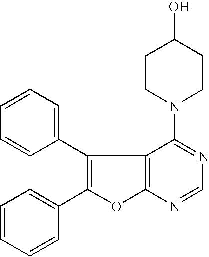 | 4.11 (B) | 372.4 | 2 |
| 237 | 598.74 | 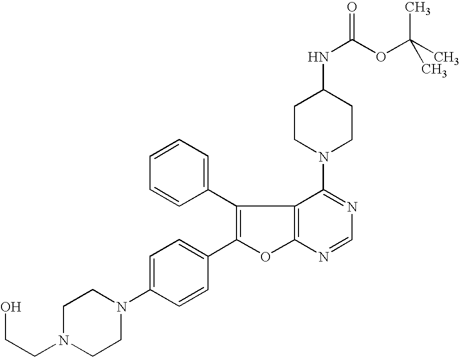 | 1.95 (E) | 599 | 14 |
| 238 | 520.03 | 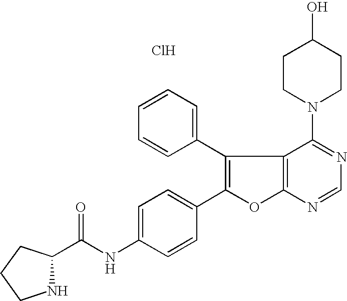 | 1.68 (E) | 484 | 5* |
| 239 | 499.61 | 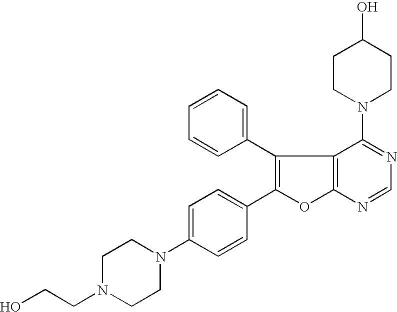 | 1.72 (E) | 500 | 14 |
| 240 | 474.54 | 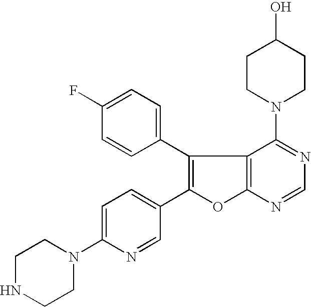 | 2.49 (B) | 475.4 | 7 |
| 241 | 488.56 | 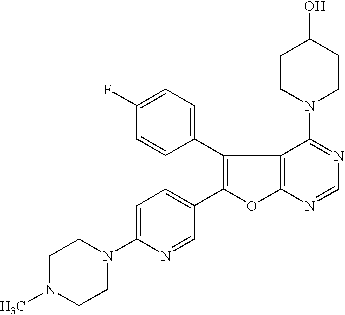 | 2.09 (E) | 489 | 7 |
| 242 | 483.61 | 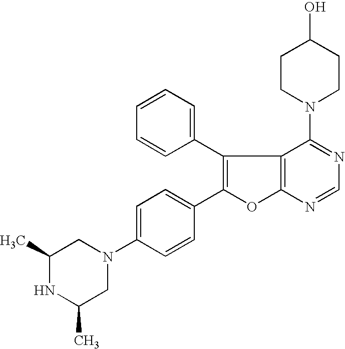 | 2.65 (B) | 484.5 | 14 |
| 243 | 485.94 | 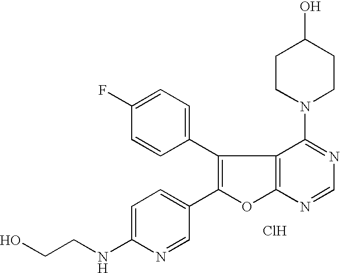 | 2.19 (E) | 450 | 7 |
| 244 | 532.64 | 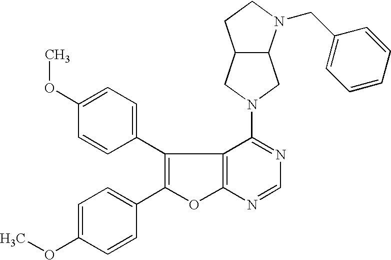 | 2.19 (E) | 533 | 2 |
| 245 | 433.48 | 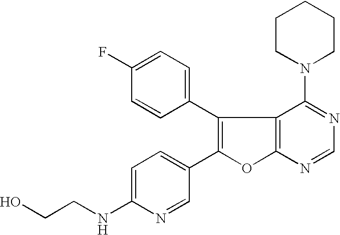 | 3.21 (B) | 434.3 | 7 |
| 246 | 447.47 | 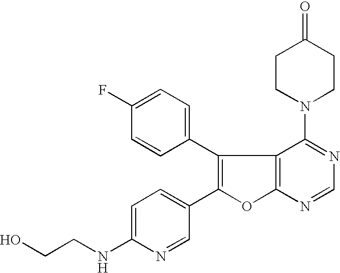 | 2.73 (B) | 448.3 | 7 |
| 247 | 509.54 | 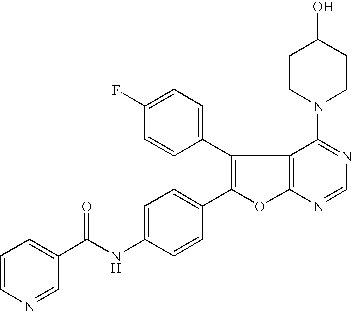 | 3.71 (A) | 510 | 5 |
| 248 | 588.68 | 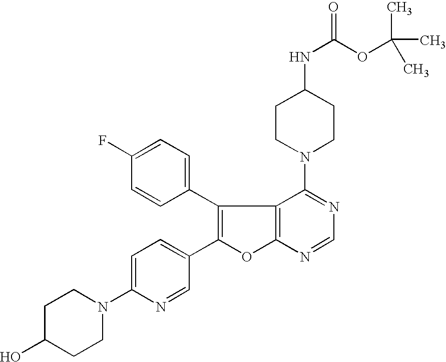 | 3.92 (B) | 589.69 | 7 |
| 249 | 676.54 | 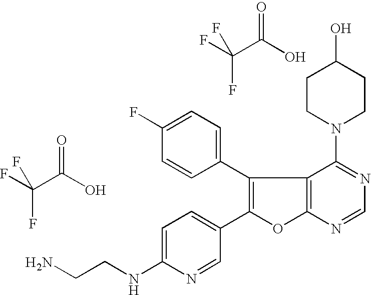 | 1.85 (E) | 449 | 7* |
| 250 | 587.70 | 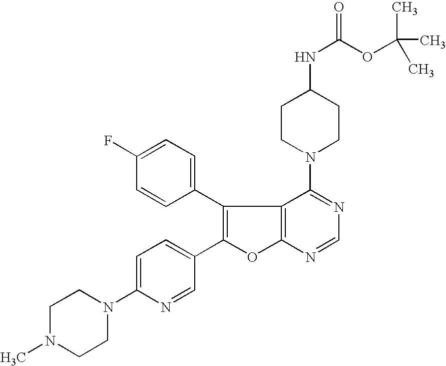 | 2.06 (E) | 588 | 7 |
| 251 | 561.49 | 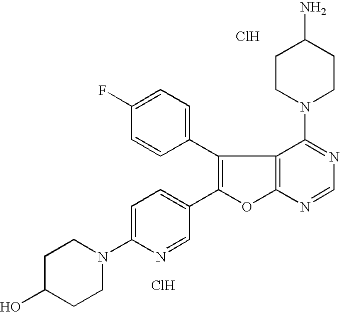 | 1.87 (E) | 489 | 7* |
| 252 | 560.50 | 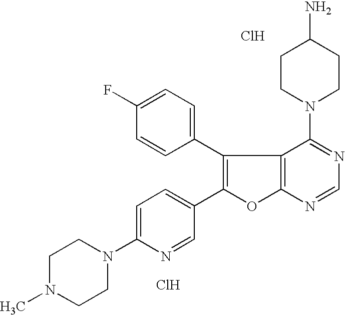 | 1.53 (E) | 265 | 7* |
| 253 | 491.53 | 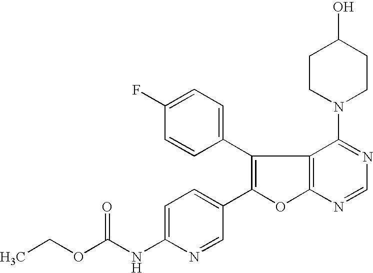 | 2.97 (B) | 491 | from example 224* |
| 254 | 531.59 | 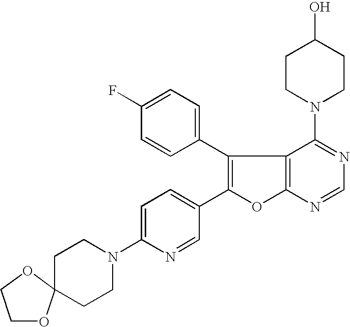 | 2.48 (E) | 532 | 7 |
| 255 | 516.57 | 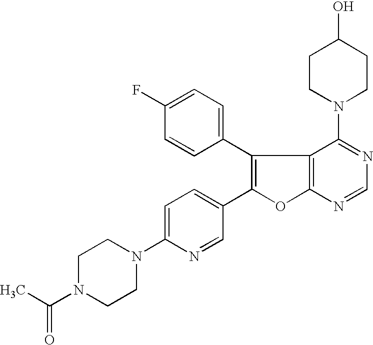 | 3.32 (B) | 517.3 | 7 |
| 256 | 473.55 | 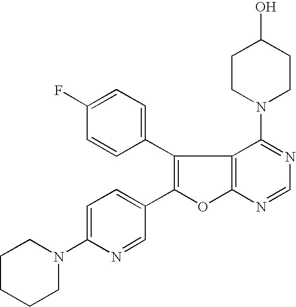 | 3.90 (B) | 474.3 | 7 |
| 257 | 459.52 | 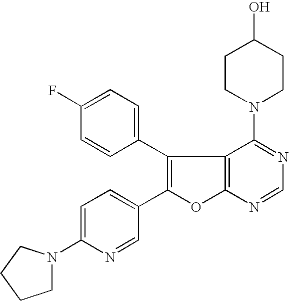 | 2.99 (B) | 460.3 | 7 |
| 258 | 530.60 | 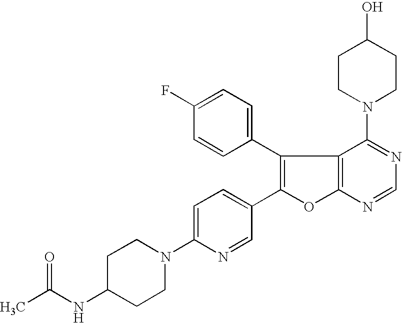 | 3.03 (B) | 531.3 | 7 |
| 259 | 458.49 | 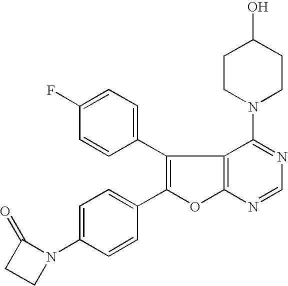 | 3.75 (B) | 459.3 | 24 |
| 260 | 422.89 | 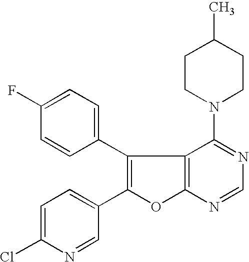 | 5.38 (B) | 432.2 | 2 |
| 261 | 439.92 | 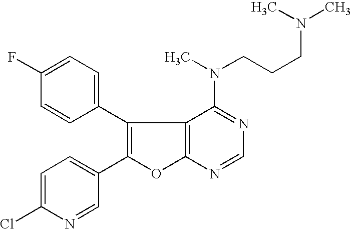 | 2.83 (B) | 440.3 | 2 |
| 262 | 502.59 | 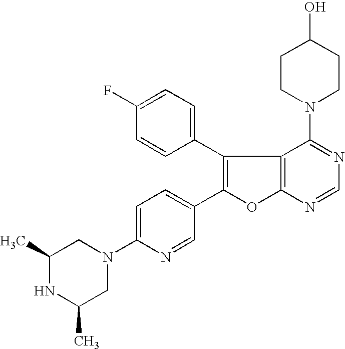 | 2.55 (B) | 503.3 | 7 |
| 263 | 687.81 | 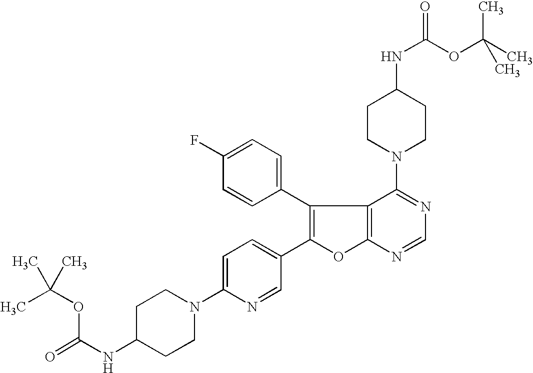 | 4.93 (B) | 688 | 7 |
| 264 | 488.56 | 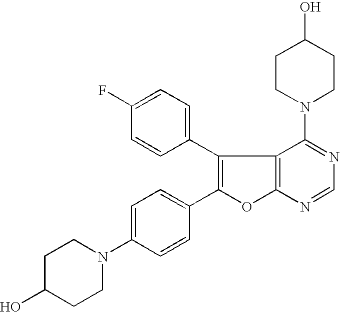 | 3.39 (B) | 489.3 | 14 |
| 265 | 471.56 | 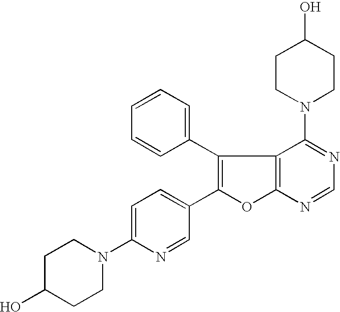 | 2.41 (B) | 472.14 | 198 |
| 266 | 575.47 | 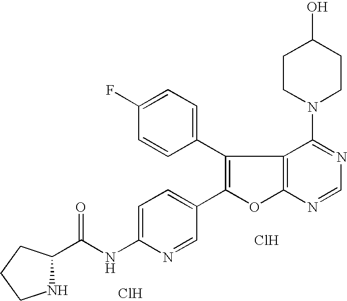 | 1.99 (B) | 503.17 | 5* |
| 267 | 487.58 | 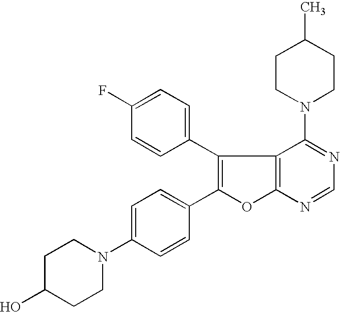 | 4.6 (B) | 488.3 | 7 |
| 268 | 487.53 | 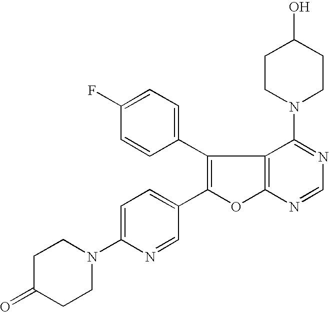 | 3.47 (A) | 488 | 7 |
| 269 | 538.02 | 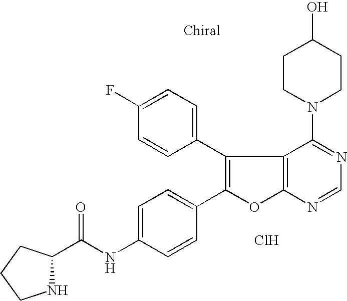 | 3.07 (B) | 502.2 | 5* |
| 270 | 596.96 | 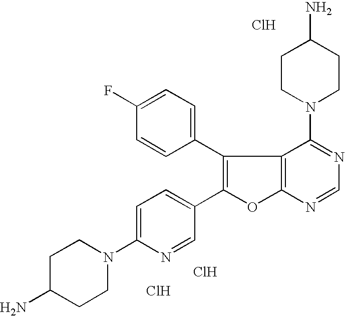 | 3.17 (A) | 488.3 | 198* |
| 271 | 445.52 | 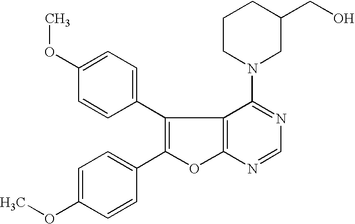 | 4.5 (B) | 446.6 | 2 |
| 272 | 541.56 | 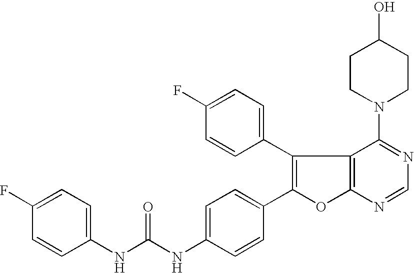 | 4.3 (B) | 542.3 | 20 |
| 273 | 575.64 | 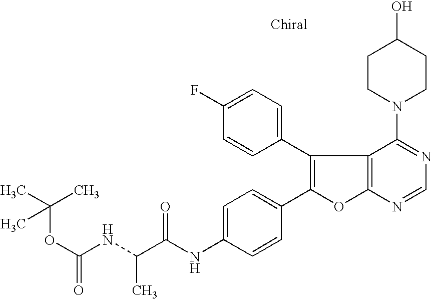 | 4.1 (B) | 567.4 | 5 |
| 274 | 431.49 | 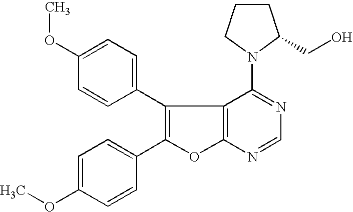 | 4.4 (B) | 432.3 | 2 |
| 275 | 433.46 | 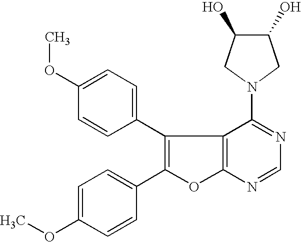 | 4.6 (B) | 434.3 | 2 |
| 276 | 431.49 | 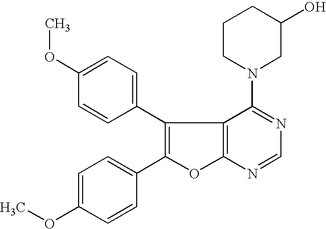 | 4.4 (B) | 432.5 | 2 |
| 277 | 459.54 | 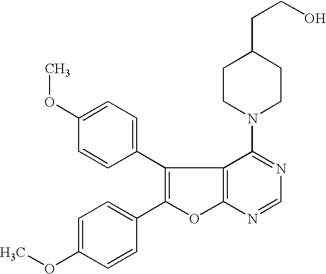 | 4.5 (B) | 460.9 | 2 |
| 278 | 445.52 | 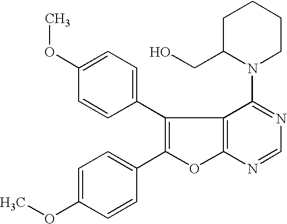 | 4.6 (B) | 446.8 | 2 |
| 279 | 403.44 | 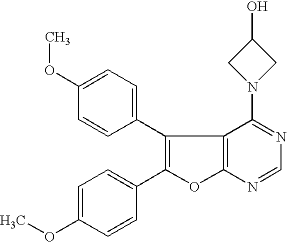 | 4.0 (B) | 404.5 | 2 |
| 280 | 498.60 | 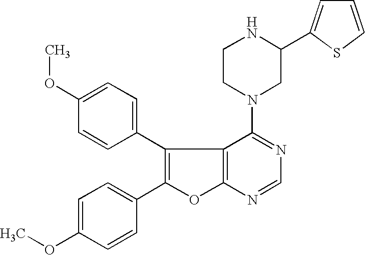 | 3.2 (B) | 499.7 | 2 |
| 281 | 548.44 | 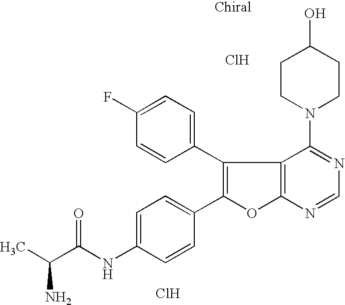 | 2.6 (B) | 476.3 | 5* |
| 282 | 458.56 | 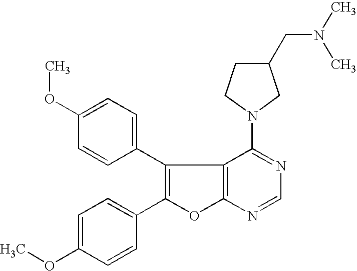 | 3.0 (B) | 459.3 | 2 |
| 283 | 531.59 | 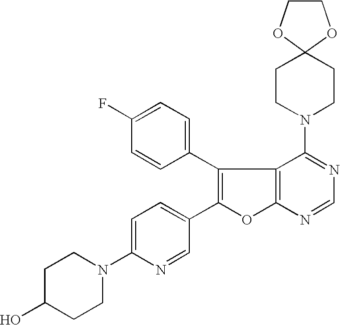 | 3.96 (B) | 532.6 | 7 |
| 284 | 573.62 | 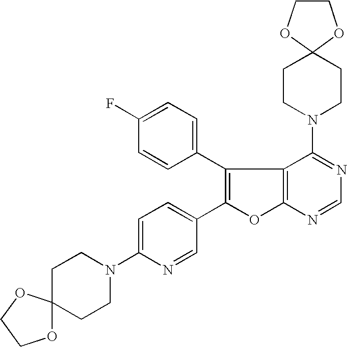 | 4.84 (B) | 574.5 | 198 |
| 285 | 592.46 | 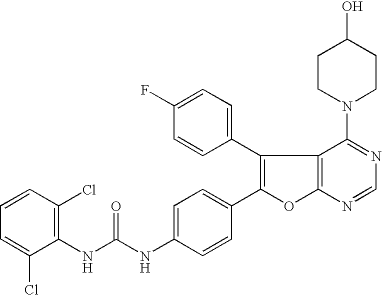 | 4.75 (B) | 592.1 | 20 |
| 286 | 487.53 | 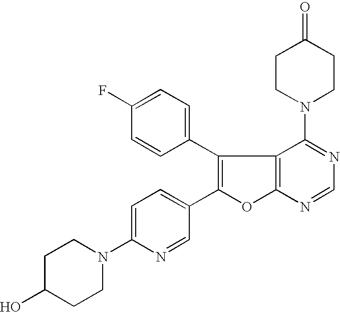 | 3.8 (B) | 488.3 | 7 |
| 287 | 501.60 | 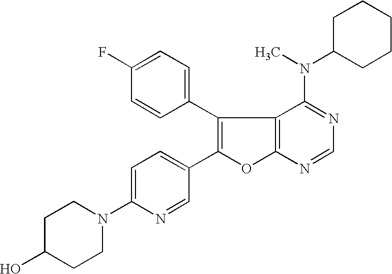 | 4.8 (B) | 502.4 | 7 |
| 288 | 572.64 | 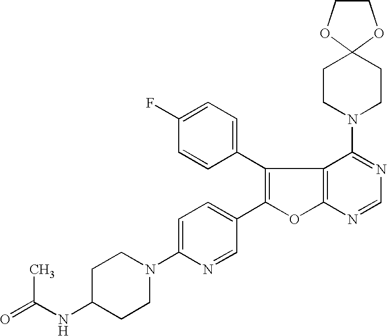 | 3.8 (B) | 573.4 | 7 |
| 289 | 558.61 | 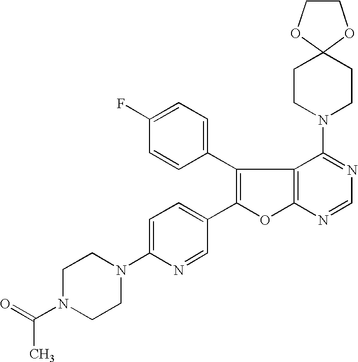 | 4.10 (B) | 559.8 | 7 |
| 290 | 541.58 | 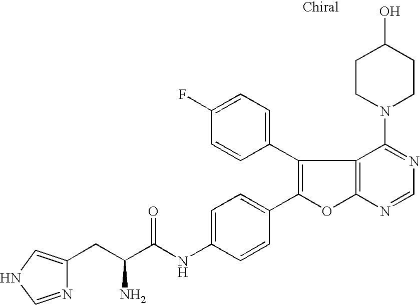 | 2.3 (B) | 542.3 | 5* |
| 291 | 498.52 | 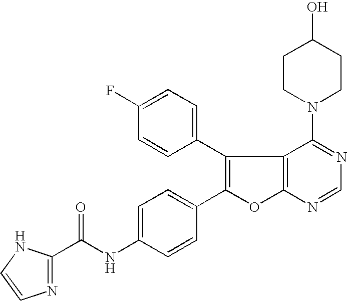 | 3.7 (B) | 499.3 | 5 |
| 292 | 615.71 | 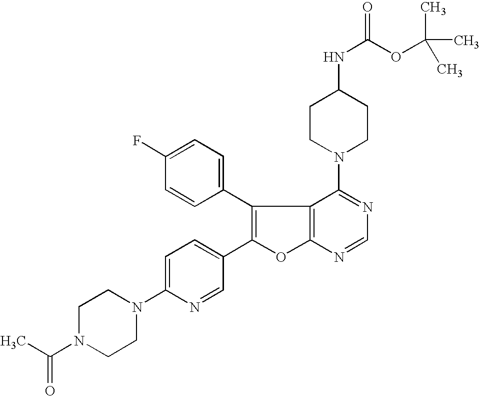 | 4.5 (B) | 616.5 | 7 |
| 293 | 558.61 | 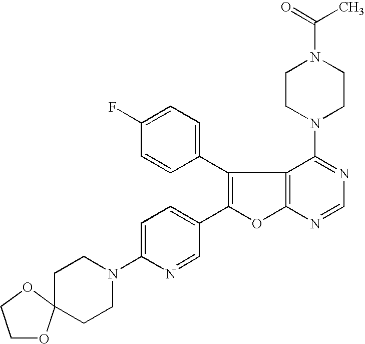 | 4.1 (B) | 559.3 | 7 |
| 294 | 544.63 | 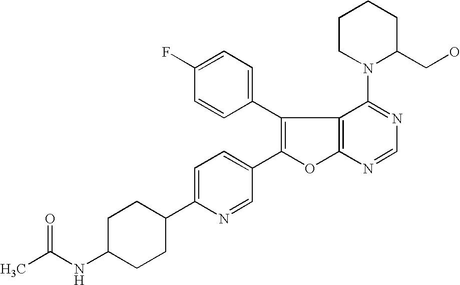 | 3.7 (B) | 545.8 | 7 |
| 295 | 516.57 | 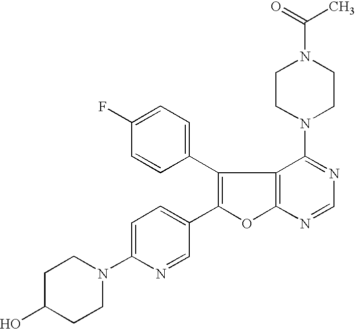 | 3.3 (B) | 517.3 | 7 |
| 296 | 557.63 | 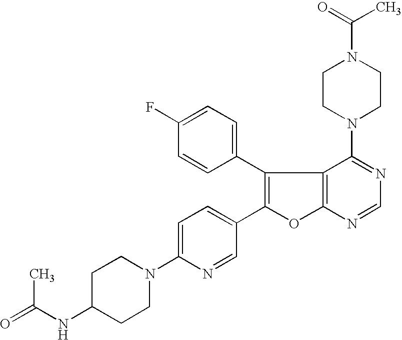 | 3.4 (B) | 558 | 7 |
| 297 | 516.57 | 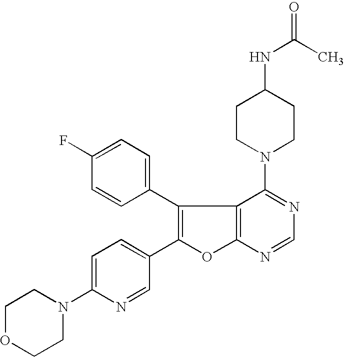 | 3.9 (B) | 517.6 | 7 |
| 298 | 572.64 | 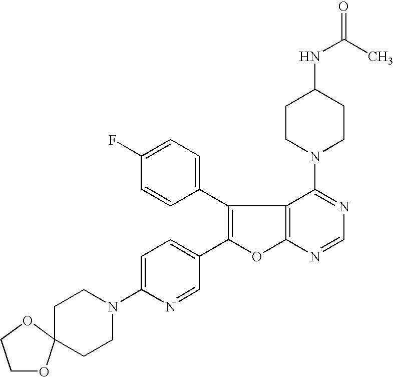 | 4.0 (B) | 573.8 | 7 |
| 299 | 517.56 | 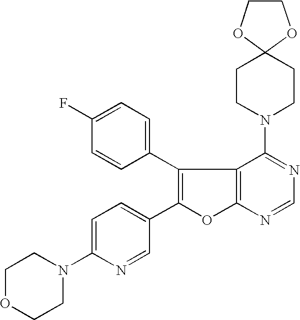 | 4.6 (B) | 518.3 | 7 |
| 300 | 530.60 | 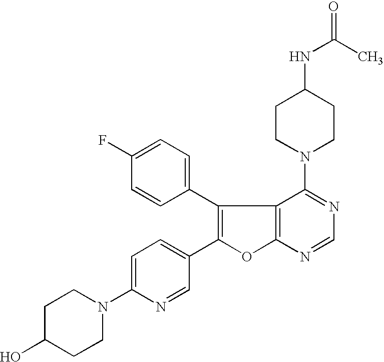 | 3.3 (B) | 531.6 | 7 |
| 301 | 505.55 | 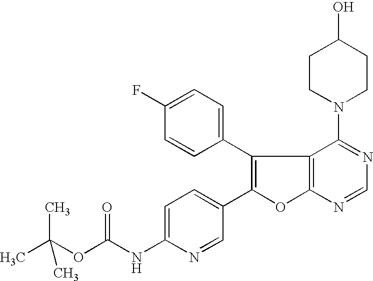 | 3.9 (B) | 506.3 | from example 224* |
| 302 | 543.60 | 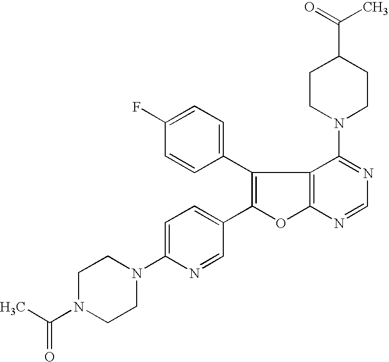 | 3.68 (B) | 544.8 | 7 |
| 303 | 557.63 | 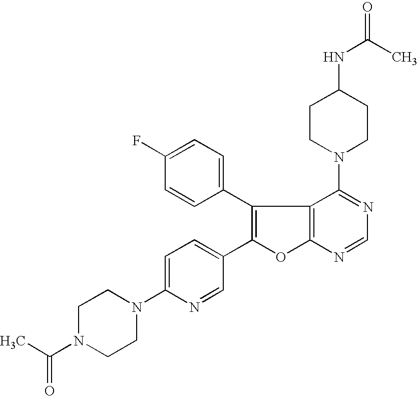 | 3.71 (B) | 558.8 | 7 |
| 304 | 629.73 | 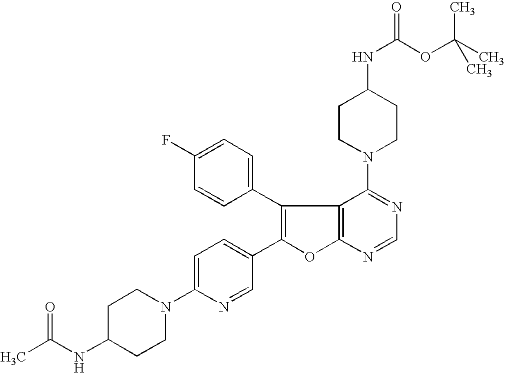 | 4.44 (B) | 630.8 | 7 |
| 305 | 528.59 | 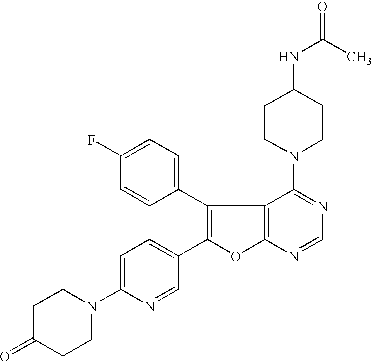 | 2.47 (B) | 529.4 | 7 |
| 306 | 473.51 | 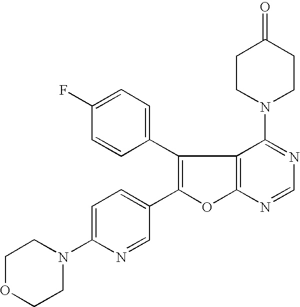 | 4.48 (B) | 474.3 | 7 |
| 307 | 502.59 | 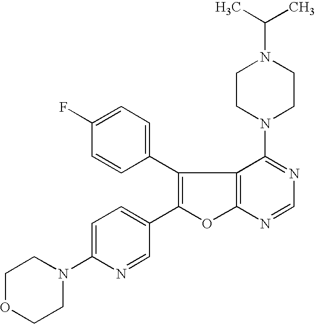 | 3.06 (B) | 503.4 | 7 |
| 308 | 543.64 | 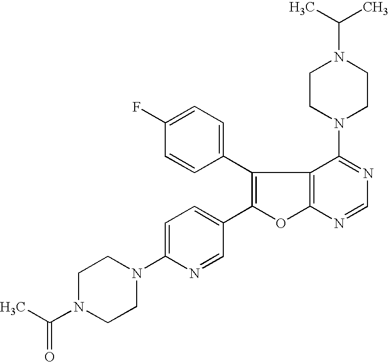 | 2.79 (B) | 544.5 | 7 |
| 309 | 502.55 | 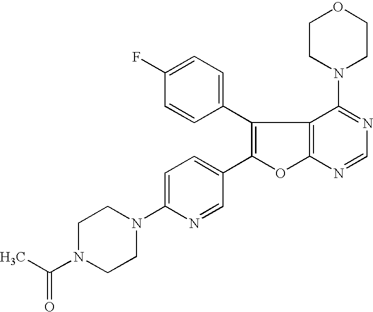 | 3.9 (B) | 503.4 | 7 |
| 310 | 442.52 | 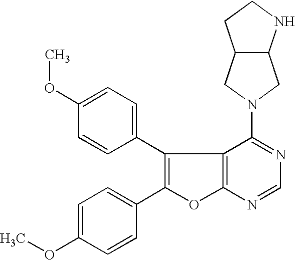 | 3.4 (B) | 443.6 | 2 |
| 311 | 523.99 | 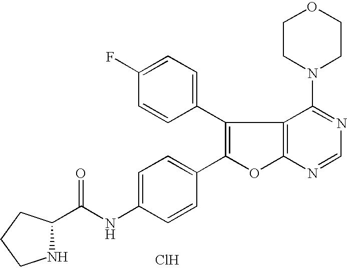 | 3.0 (B) | 488.4 | 5* |
| 312 | 511.00 | 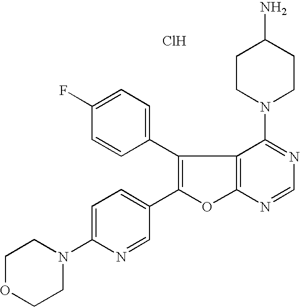 | 3.40 (A) | 475.2 | 7* |
| 313 | 553.04 | 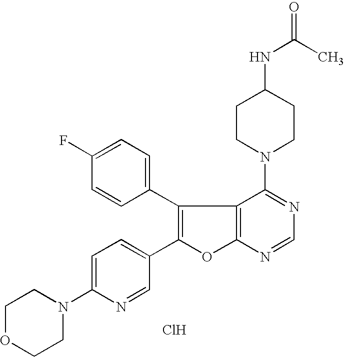 | 4.0 (B) | 517.7 | 195* |
| 314 | 461.50 | 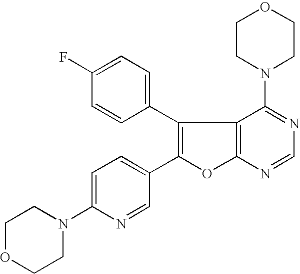 | 2.80 (B) | 462.3 | 195 |
| 315 | 539.05 | 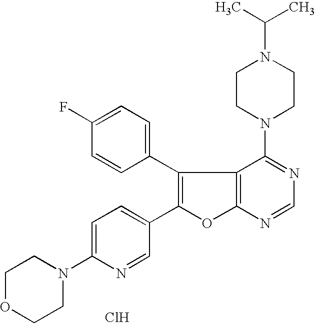 | 3.1 (B) | 503.4 | 196 |
| 316 | 580.06 | 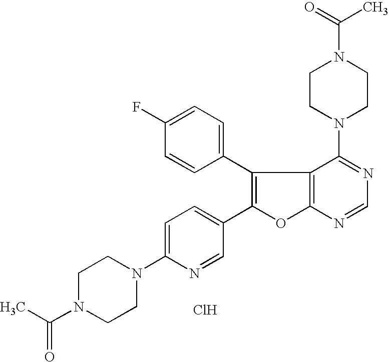 | 3.7 (B) | 544.7 | 196 |
| 317 | 567.06 | 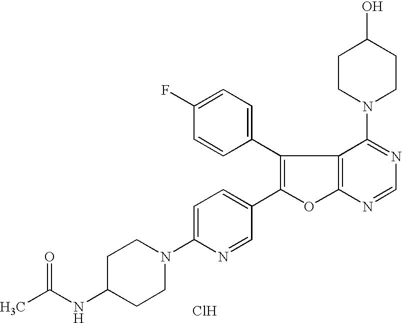 | 3.5 (B) | 531.7 | 196 |
| 318 | 605.54 | 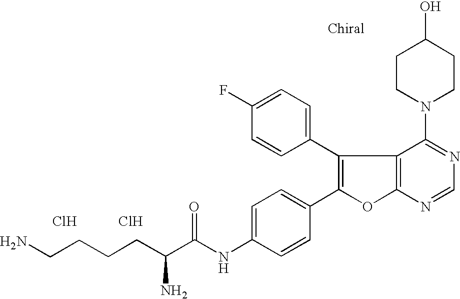 | 0.40 (B) | 533.3 | 5* |
| 319 | 591.51 | 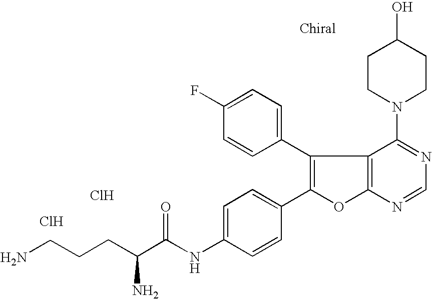 | 0.39 (B) | 519.3 | 5* |
| 320 | 526.01 | 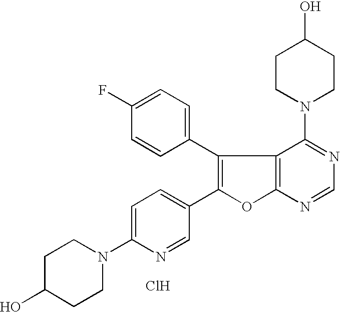 | 3.50 (A) | 490.2 | 196 |
| 321 | 527.98 | 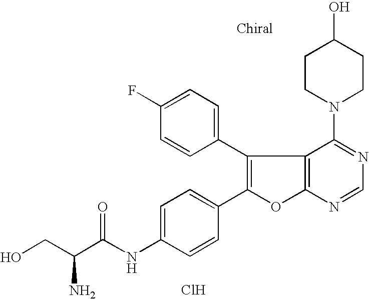 | 0.34 (B) | 492.2 | 5* |
| 322 | 552.09 | 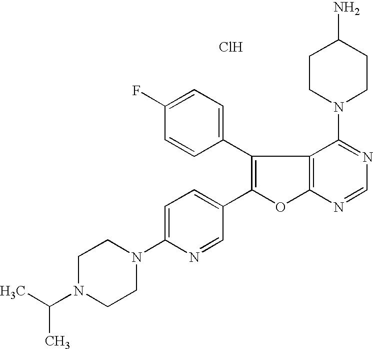 | 2.7 (B) | 516.3 | 7* |
| 323 | 547.10 | 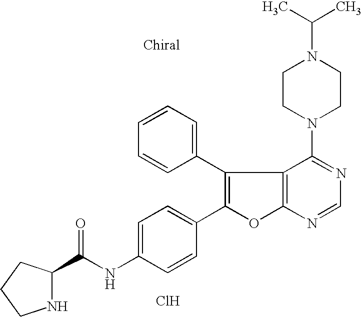 | 3.69 (A) | 511.3 | 5* |
| 324 | 463.38 | 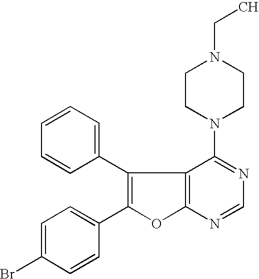 | 3.6 (B) | 463 [M]⁺ | 2 |
| 325 | 502.59 | 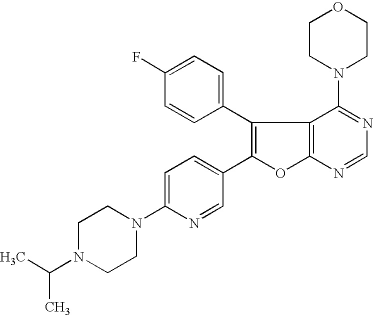 | 3.90 (A) | 503.4 | 7 |
| 326 | 515.63 | 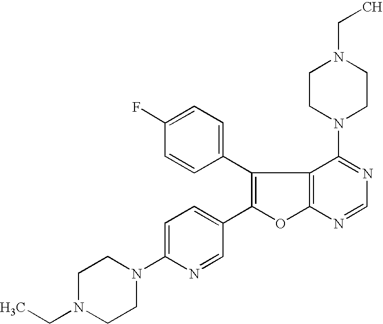 | 2.5 (B) | 516.4 | 198 |
| 327 | 502.59 | 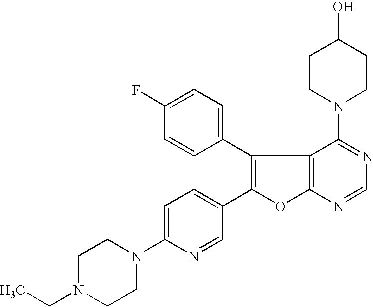 | 2.8 (B) | 503.8 | 7 |
| 328 | 558.65 | 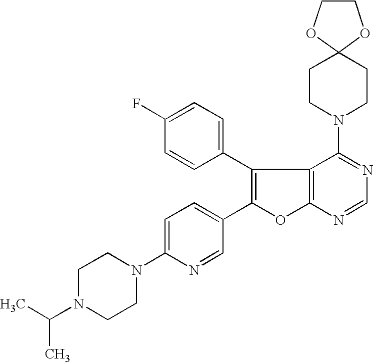 | 1.81 (B) | 559.4 | 7 |
| 329 | 596.73 | 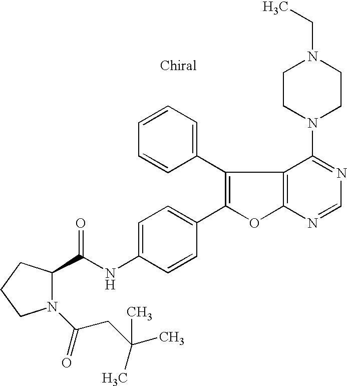 | 3.4 (B) | 597.8 | 5 |
| 330 | 533.07 | 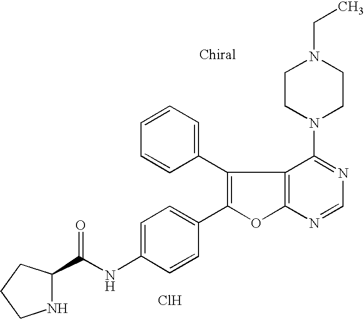 | 2.25 (B) | 497.3 | 5* |
| 331 | 547.14 | 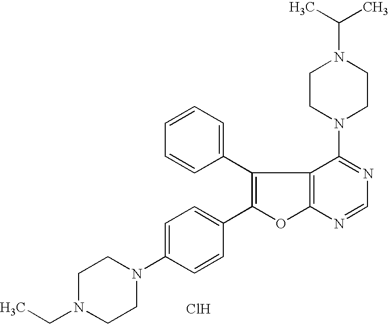 | 3.70 (A) | 511.2 | 197 |

### Notes on the Preparation of Examples 34-184 and 205-331:

### Example 42

The 2-methylbenzoxazole-5-carboxylic acid used can be prepared from 3-amino-4-hydroxybenzoic acid analogously to the following procedure: Nagano et al., J. Am. Chem. Soc. 1953, 75, 6237.

### Example 86/92

The 2-methyltetrahydrofuran-2-carboxylic acid used can be prepared from 5-chloropentan-2-one analogously to the following procedure: Justoni et al., Gazz. Chim. Ital. 1950, 80, 259.

### Examples 100/107

The isocyanates used, 1-isocyanato-3-methylcyclohexane and 2-isocyanato-1,3-dimethylcyclohexane, can be obtained from 3-methylcyclohexylamine and 2,6-dimethylcyclohexylamine, respectively, by reaction with phosgene analogously to the following procedure: J. Pharm. Pharmacol., 1964, 16, 538.

### Examples 142/155/163/164/219/230/231/238/251/252/266/269/270/281/290/311/-312/318/319/321/322/323/330:

The examples were prepared from the corresponding N-Boc-protected derivatives by removing the Boc group according to standard methods (see: T. Greene, P. Wuts in Protective Groups in Organic Synthesis, 1999, J. Wiley and Sons, New York).

### Example 158

The 5-methylaminomethyl-furan-2-ylmethanol used can be prepared from 5-hydroxymethylfuran-2-carbaldehyde by reductive amination analogously to the following procedure: Müller et al., Tetrahedron 1998, 54, 10703.

### Example 166

The synthesis was carried out by ether cleavage of example 169 by heating under reflux in a 2:1 mixture of 48% strength hydrobromic acid and acetic acid for 16 hours (yield: 61%), analogously to literature procedures.

### Example 167

The compound was prepared from the corresponding N-benzyl-protected derivative by removing the benzyl group according to standard methods (see: T. Green, P. Wuts in Protective Groups in Organic Synthesis, 1999, J. Wiley and Sons, New York).

### Example 170

The synthesis was carried out by reacting intermediate 11a in a Stille coupling with 3-(trimethylstannyl)pyridine as tin reagent according to standard procedures (yields: 9%) (see: V. Farina, V. Krishnamurthy, W. J. Scott in: The Stille Reaction, 1998, J. Wiley and Sons, New York).

### Example 249

The purification was carried out as described in example 7 by RP-HPLC, but with addition of 0.1% trifluoroacetic acid.

### Examples 253/301

The synthesis was carried out by reacting the stated intermediates with the corresponding acids according to standard procedures (see: T. Greene, P. Wuts in Protective Groups in Organic Synthesis, 1999, J. Wiley and Sons, New York).

### Example 313

The synthesis was carried out by reacting the HCl-free compound with a solution of HCl in dioxane and subsequent removal of the solvent.

### HPLC Methods for Examples 34-184 and 205-331

(A): Mobile phase A: 0.5% HClO₄ in water; Mobile phase B: acetonitrile; Gradient: 0.5 min 98% A, 2% B; 4.5 min 10% A, 90% B; 6.7 min 98% A, 2% B; Flow rate: 0.75 ml/min; Column temperature: 30° C.; UV detection at 210 nm; Column: Kromasil C18 (60×2 mm).

(B): Mobile phase A: 0.1% formic acid in water; Mobile phase B: 0.1% formic acid in acetonitrile; Gradient: 0 min 90% A, 10% B; 4 min 10% A, 90% B; 6.1 min 90% A, 10% B; Flow rate: 0.5 ml/min; Column temperature: 40° C.; UV detection at 210 nm; Column: Symmetry C18 (150×2.1 mm).

(C): Mobile phase A: 0.06% HCl in water; Mobile phase B: acetonitrile; Gradient: 1 min 90% A, 10% B; 4 min 10% A, 90% B; Flow rate: 0.6 ml/min; Column temperature: 50° C.; UV detection at 210 nm; Column: Symmetry C18 (10×2.1 mm).

(D): As for method (A), except: Gradient: 0.5 min 98% A, 2% B; 4.5. min 10% A, 90% B; 9.2 min 98% A, 2% B.

(E): Mobile phase A: 0.01% HCl in water; Mobile phase B: acetonitrile; Gradient: 0 min 98% A, 2% B, 2.5 min 5% A, 95% B; Flow rate: 0.9-1.2 ml/min; Column temperature: 70° C.; UV detection at 210 nm; Column: Symmetry C18 (150×2.1 mm).

1. A compound of the formula (I) 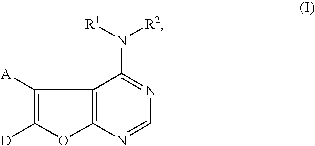
in which
A represents phenyl or 5- or 6-membered heteroaryl having up to three heteroatoms from the group consisting of N, O and/or S, each of which radicals may be substituted up to three times, independently of one another, by substituents from the group consisting of halogen, hydroxyl, (C₁-C₆)-alkoxy, trifluoromethyl, trifluoromethoxy, amino, carboxyl and (C₁-C₆)-alkoxycarbonyl, or represents a group of the formula 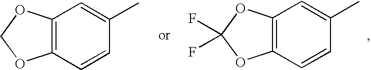
D represents a group of the formula
R³-E-G
in which
G represents phenylene or 5- or 6-membered heteroarylene having up to three heteroatoms from the group consisting of N, O and/or S, each of which radicals may be substituted up to two times, independently of one another, by substituents from the group consisting of halogen, trifluoromethyl, trifluoromethoxy, (C₁-C₆)-alkoxy, amino, nitro and carboxyl,
E represents a bond, a carbonyl group, a sulfonyl group or represents a group of the formula *—C(O)—NR⁴— or *—SO₂—NR⁴—,
in which * denotes the point of attachment to the group R³ and R⁴ represents hydrogen or (C₁-C₆)-alkyl,
and
R³ represents halogen, trifluoromethyl, hydroxyl, optionally hydroxyl- or amino-substituted (C₁-C₆)-alkoxy, trifluoromethoxy, nitro, carboxyl or a group of the formula H—C(O)—NR⁴—,
in which R⁴ is as defined above,
represents (C₁-C₆)-alkyl which may be mono- to disubstituted, independently of one another, by substituents selected from the group consisting of halogen, trifluoromethyl, hydroxyl (C₁-C₆)-alkoxy, amino, mono- or di-(C₁-C₆)-alkylamino, (C₁-C₆)-acylamino, (C₁-C₆)-alkoxycarbonylamino, amidino, guanidino, carboxyl, (C₁-C₆)-alkoxycarbonyl, (C₆-C₁₀)-aryl, (C₆-C₁₀)-aryloxy and 5- to 10-membered heteroaryl having up to three heteroatoms from the group consisting of N, O and/or S, where aryl, aryloxy and heteroaryl for their part may in each case be mono- to disubstituted, independently of one another, by halogen, hydroxyl, amino, (C₁-C₄)-alkyl, (C₁-C₆)-alkoxy, cyano or nitro,
represents (C₃-C₇)-cycloalkyl which may be substituted by phenyl or up to four times by (C₁-C₄)-alkyl,
represents (C₆-C₁₀)-aryl, which may be mono- to disubstituted, independently of one another, by substituents selected from the group consisting of halogen, trifluoromethyl, (C₁-C₆)-alkyl, hydroxyl, (C₁-C₆)-alkoxy, (C₁-C₆)-alkanoyl, cyano, nitro, amino, mono- and di-(C₁-C₆)-alkylamino, (C₁-C₆)-acylamino, carboxyl, (C₁-C₆)-alkoxycarbonyl and 5- to 6-membered heteroaryl having up to two heteroatoms from the group consisting of N, O and/or S,
represents 4- to 7-membered, saturated or partially unsaturated heterocyclyl which is attached via a ring carbon atom or via a ring nitrogen atom and has up to three heteroatoms from the group consisting of N, O and/or S, which may be substituted up to three times, independently of one another, by (C₁-C₆)-alkyl, which for its part may be substituted by hydroxyl, (C₁-C₄)-alkoxy or phenyl, (C₁-C₆)-alkoxy-carbonyl, (C₁-C₆)-alkoxycarbonylamino, (C₁-C₆)-alkanoyl, (C₁-C₆)-alkylcarbonylamino, 1,2-dioxyethylene, carboxyl, amino, hydroxyl, (C₁-C₆)-alkoxy or an oxo group,
represents 5- to 10-membered heteroaryl which is attached via a ring carbon atom or via a ring nitrogen atom and has up to three heteroatoms from the group consisting of N, O and/or S, which for its part may optionally be mono- to disubstituted, independently of one another, by substituents selected from the group consisting of halogen, nitro, amino, hydroxyl (C₁-C₆)-alkyl, (C₃-C₆)-cycloalkyl, phenyl, benzyl and 5-membered heteroaryl having up to two heteroatoms from the group consisting of N, O and/or S,
or
represents a group of the formula —NR⁵R⁶,
in which
R⁵ and R⁶ independently of one another represent hydrogen, (C₁-C₆)-alkyl, which may be substituted by hydroxyl, amino, (C₁-C₄)-alkoxy, mono- or di-(C₁-C₄)-alkylamino or phenyl, represent (C₃-C₇)-cycloalkyl, which may be mono- to disubstituted, independently of one another, by hydroxyl, amino, (C₁-C₄)-alkoxy, mono- or di-(C₁-C₄)-alkylamino or (C₁-C₄)-alkyl, represent (C₆-C₁₀)-aryl, which may be mono-to disubstituted, independently of one another, by hydroxyl, halogen, amino, (C₁-C₄)-alkoxy or nitro, or represent 5- to 6-membered heterocyclyl or 5- to 6-membered heteroaryl having in each case up to two heteroatoms from the group consisting of N, O and/or S,
or
D represents a group of the formula 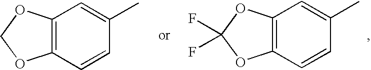
R¹ represents hydrogen, (C₃-C₆)-cycloalkyl or represents (C₁-C₆)-alkyl which may be mono- to disubstituted, independently of one another, by hydroxyl (C₁-C₆)-alkoxy, amino, mono- or di-(C₁-C₆)-alkylamino,
and
R² represents (C₁-C₆)-alkyl, which may be mono- to disubstituted, independently of one another, by substituents selected from the group consisting of trifluoromethyl, hydroxyl, (C₁-C₆)-alkoxy, amino, mono- or di-(C₁-C₆)-alkylamino, phenylamino, carboxyl, (C₁-C₆)-alkoxycarbonyl, (C₃-C₇)-cycloalkyl, phenyl, which for its part is optionally mono- to disubstituted by (C₁-C₄)-alkoxy, 5- to 6-membered heterocyclyl having up to two heteroatoms from the group consisting of N, O and/or S and 5- to 6-membered heteroaryl having up to two heteroatoms from the group consisting of N, O and/or S, which for its part is optionally substituted by (C₁-C₄)-alkyl or hydroxy-(C₁-C₄)-alkyl,
represents (C₆-C₁₀)-aryl which may be substituted by hydroxyl (C₁-C₆)-alkoxy, amino, mono or di-(C₁-C₆)-alkylamino,
represents 5- to 6-membered heteroaryl having up to three heteroatoms from the group consisting of N, O and/or S which may be substituted by (C₁-C₆)-alkyl, trifluoromethyl, halogen, hydroxyl, (C₁-C₆)-alkoxy, trifluoromethoxy, amino, mono- or di-(C₁-C₆)-alkylamino,
represents 5- to 6-membered heterocyclyl having up to two heteroatoms from the group consisting of N, O and/or S which may be substituted by benzyl or up to four times by (C₁-C₄)-alkyl,
or
represents (C₄-C₈)-cycloalkyl which may be mono- to disubstituted, independently of one another, by hydroxyl amino, (C₁-C₆)-alkyl, (C₁-C₆)-alkoxy, mono- or di-(C₁-C₆)-alkylamino or a group of the formula R⁷—C(O)—NH— or R⁷—SO₂—NH—,
in which
R⁷ represents (C₁-C₆)-alkoxy, phenyl, benzyl, phenylamino, mono- or di-(C₁-C₆)-alkylamino, which for their part are optionally substituted in the alkyl group by (C₁-C₄)-alkoxycarbonyl or carboxyl, or represents (C₄-C₇)-cycloalkylamino which for its part is optionally substituted in the cycloalkyl group by (C₁-C₄)-alkyl,
represents (C₁-C₆)-alkyl which may be substituted by hydroxyl, (C₁-C₆)-alkoxy, amino, mono or di-(C₁-C₆)-alkylamino, (C₁-C₆)-acylamino or by 5- or 6-membered heterocyclyl having up to two heteroatoms from the group consisting of N, O and/or S,
represents 5- or 6-membered heterocyclyl which is attached via a ring carbon atom or via a ring nitrogen atom and has up to two heteroatoms from the group consisting of N, O and/or S, which is optionally substituted by an oxo group,
or
represents 5- or 6-membered heteroaryl having up to three heteroatoms from the group consisting of N, O and/or S, which is optionally mono- to disubstituted by (C₁-C₄)-alkyl,
or
R¹ and R² together with the nitrogen atom to which they are attached form a 4- to 11-membered mono-, bi- or spiro-cyclic heterocycle which may contain up to two further heteroatoms from the group consisting of N, O and/or S and which may be mono- to tetrasubstituted, independently of one another, by substituents selected from the group consisting of amino, mono- or di-(C₁-C₆)-alkylamino, hydroxyl (C₁-C₆)-alkoxy, oxo, carboxyl, carbamoyl, (C₁-C₆)-alkoxycarbonyl, (C₁-C₆)-alkoxycarbonylamino, (C₁-C₆)-alkanoyl, (C₁-C₆)-alkylcarbonylamino, (C₁-C₆)-alkyl, which for its part is optionally substituted by hydroxyl, (C₁-C₆)-alkoxy, amino, mono or di-(C₁-C₆)-alkylamino, phenyl or by 5- or 6-membered heterocyclyl having up to two heteroatoms from the group consisting of N, O and/or S, phenyl which for its part is optionally substituted by halogen, (C₃-C₇)-cycloalkyl, pyridyl, thienyl and 5- or 6-membered heterocyclyl having up to two heteroatoms from the group consisting of N, O and/or S,
or a pharmaceutically acceptable salt, solvate, hydrate or hydrate of a salt thereof

2. A compound as claimed in claim 1 of the formula (Ia) 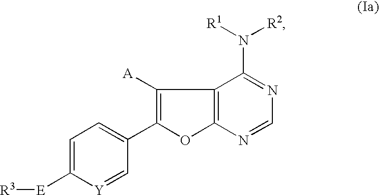
in which
A represents phenyl which may be substituted by fluorine, chlorine, bromine or methoxy,
Y represents CH or N,
E represents a bond, represents a carbonyl group or represents a group of the formula *—C(O)—NH—,
in which * denotes the point of attachment to the group R³,
R³ represents hydroxyl, methoxy, amino or mono-(C₁-C₄)-alkylamino, which may be substituted in the alkyl group by hydroxyl or amino,
represents (C₁-C₄)-alkyl which is optionally substituted by hydroxyl methoxy, ethoxy, amino, mono or dimethylamino, (C₁-C₄)-alkoxycarbonylamino, acetamido, carboxyl or (C₁-C₄)-alkoxycarbonyl,
or
represents a 4- to 6-membered, saturated heterocycle which is attached via a ring carbon atom or via a ring nitrogen atom and has up to two heteroatoms from the group consisting of N and/or O, which may be substituted up to two times, independently of one another, by (C₁-C₄)-alkyl, which for its part may be substituted by hydroxyl methoxy or ethoxy, by carboxyl, amino, hydroxyl methoxy, ethoxy or an oxo group,
R¹ represents hydrogen, methyl or represents ethyl which may be substituted by hydroxyl or amino,
and
R² represents (C₁-C₄)-alkyl which may be mono- to disubstituted, independently of one another, by hydroxyl, amino, (C₁-C₄)-alkoxy, mono- or di-(C₁-C₄)-alkylamino, or represents (C₅-C₇)-cycloalkyl
or
R¹ and R² together with the nitrogen atom to which they are attached form a pyrrolidine, piperidine, piperazine, or morpholine ring which may be mono- or disubstituted, independently of one another, by (C₁-C₄)-alkyl, which for its part is optionally substituted by hydroxyl or amino, by amino, mono or di-(C₁-C₄)-alkylamino, hydroxyl, (C₁-C₄)-alkoxy, oxo, carboxyl, carbamoyl or by (C₁-C₄)-alkylcarbonyl.
or a pharmaceutically acceptable salt, solvate, hydrate or hydrate of a salt thereof.

3. A compound as claimed in claim 1 of the formula (Ia) in which
A represents phenyl which can be substituted by fluorine,
Y represents CH or N,
E represents a bond or represents a group of the formula *—C(O)—NH—,
in which*denotes the point of attachment to the group R³,
R³ represents amino or mono-(C₁-C₄)-alkylamino which may be substituted in the alkyl group by hydroxyl or amino,
represents (C₁-C₄)-alkyl which is optionally substituted by hydroxyl, amino, mono- or dimethylamino,
or
represents pyrrolidine, piperazine or piperidine which may be substituted up to two times, independently of one another, by methyl, ethyl, n-propyl, isopropyl, n-butyl, sec-butyl, isobutyl or tert-butyl, which for their part may be substituted by hydroxyl, by amino or hydroxyl,
R¹ and R² together with the nitrogen atom to which they are attached form a piperidine, piperazine or morpholine ring which may be mono- or disubstituted, independently of one another, by methyl, ethyl, n-propyl or isopropyl, which for their part are optionally substituted by hydroxyl or amino, by amino, hydroxyl or oxo,
or a pharmaceutically acceptable salt, solvate, hydrate or hydrate of a salt thereof.

4. A process for preparing the compounds of the formula (I), as defined in claim 1, characterized in that
compounds of the formula (II) 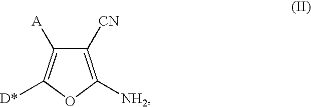
in which D* represents D or an unsubstituted phenyl ring and A and D are as defined in claim 1
are either
[A] initially reacted with formic acid in acetic anhydride to give compounds of the formula (III) 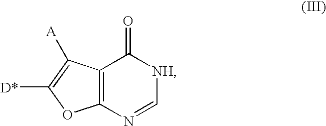
in which D* represents D or represents an unsubstituted phenyl ring and A and D are as defined in claim 1,
then converted with phosphoryl chloride into compounds of the formula (IV) 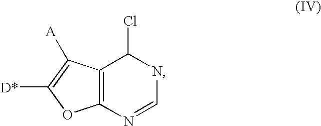
in which D* represents D or represents an unsubstituted phenyl ring and A and D are as defined in claim 1,
if D* represents an unsubstituted phenyl ring, this phenyl ring is then nitrated, and are finally reacted with compounds of the formula (V) 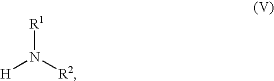
in which R¹ and R² are as defined in claim 1,
to compounds of the formula (I)
or
[B] initially converted with triethyl orthoformate into compounds of the formula (VI) 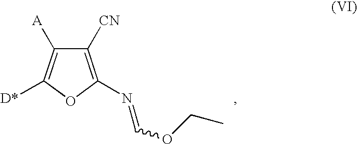
in which D* represents D or represents an unsubstituted phenyl ring and A and D are as defined in claim 1,
and then reacted with compounds of the formula (VII) 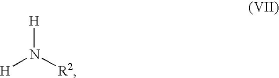
in which R² is as defined in claim 1,
and then reacted directly or, if D* represents an unsubstituted phenyl ring, with a base by subsequent nitration of this phenyl ring, to give compounds of the formula (I),
where the resulting compounds of the formula (I) can optionally, subsequently be subjected to further derivatizations which can be carried out by customary methods.

5. (cancelled)

6. A pharmaceutical composition, comprising at least one compound of the formula (I) as defined in claim 1 and at least one further auxiliary.

7. A pharmaceutical composition, comprising at least one compound of the formula (I) as defined in claim 1 and at least one further active compound.

8. A method for preventing or treating ischemia-related peripheral and cardiovascular disorders, comprising administering to a mammal an effective amount of a compound of formula (I) as defined in claim 1.

9. A method for the acute and chronic treatment of ischemic disorders of the cardiovascular system, comprising administering to a mammal an effective amount of a compound of formula (I) as defined in claim 1.

10. A method for treating acute or chronic pain and neurodegenerative disorders, comprising administering to a mammal an effective amount of a compound of formula (I) as defined in claim 1.

11. The method of claim 9, wherein the ischemic disorders of the cardiovascular system are coronary heart disease, stable and unstable angina pectoris, peripheral and arterial occlusion diseases, thrombotic vascular occlusions, myocardial infarction and reperfusion damage.
